(19) United States
(12) Patent Application Publication
(10) Pub. No.: US 2009/0143361 A1
(43) Pub. Date: Jun. 4, 2009

(54) Pyrido[3,2-E]Pyrazines, Process For Preparing The Same, And Their Use As Inhibitors Of Phosphodiesterase 10

(75) Inventors: Michael S. Malamas, Jamison, PA (US); Yike Ni, Monmouth JCT, NJ (US); James Joseph Erdei, Flourtown, PA (US); Hans Stange, Riesa (DE); Rudolf Schindler, Dresden (DE); Norbert Hofgen, Ottendorf-Okrilla (DE); Ute Egerland, Radebeul (DE); Barbara Langen, Radebeul (DE)

Correspondence Address: FISH & RICHARDSON P.C., P.O BOX 1022, MINNEAPOLIS, MN, 55440-1022, US

(73) Assignees: ELBION GMBH, Radebeul (DE); WYETH, Madison, NJ (US)

(21) Appl. No.: 12/277,961

(22) Filed: Nov. 25, 2008

Related U.S. Application Data
(60) Provisional application No. 61/004,954, filed on Nov. 30, 2007.

(51) Int. Cl. A61K 31/5377; C07D 471/14; A61K 31/4985; C07D 243/14; A61K 31/55; A61P 25/18; A61P 25/14; A61P 25/28; A61P 25/00; A61P 25/06; A61P 29/00; A61P 3/04; A61P 3/10; A61P 25/24; A61P 25/16; A61P 25/22; C07D 223/22; A61K 31/5513; C07D 401/14; C07D 413/14

(52) U.S. Cl. 514/217; 544/346; 514/250; 544/295; 544/115; 514/233.2; 540/570; 514/221; 540/589

(57) ABSTRACT

The invention relates to pyrido[3,2-e]pyrazines, to processes for preparing them, to pharmaceutical compositions which comprise these compounds and to the pharmaceutical use of these compounds, which are inhibitors of phosphodiesterase 10, as active compounds for treating central nervous system disorders, obesity, and metabolic disorders.

### CROSS REFERENCE TO RELATED APPLICATIONS

This application claims the benefit of U.S. Provisional Patent Application Ser. No. 61/004,954, filed Nov. 30, 2007, the disclosure of which is incorporated herein by reference in its entirety.

### FIELD OF THE INVENTION

The invention relates to pyrido[3,2-e]pyrazines, which are inhibitors of phosphodiesterase 10 and useful for treating diseases related to the central nervous system as well as obesity and metabolic disorders.

### BACKGROUND

Psychotic disorders, especially schizophrenia, are severe mental disorders which extremely impair daily life. The symptoms of psychosis may be divided into two fractions. In the acute phase, it is predominated by hallucinations and delusions being called the positive symptoms. When the agitated phase abates the so called negative symptoms become obvious. They include cognitive deficits, social phobia, reduced vigilance, indifference and deficits in verbal learning and memory, verbal fluency and motor function.

Although several antipsychotics are available since, the present therapy of psychosis is not satisfactory. The classic antipsychotics, such as haloperidol, with a high affinity to dopamine D2 receptor show extreme side effects, such as extrapyramidal symptoms (=EPS) and do not improve the negative symptoms of schizophrenia so that they do not enable the patient to return to everyday life.

Clozapine which has emerged as a benchmark therapeutic ameliorating positive, negative and cognitive symptoms of schizophrenia and devoid of EPS shows agranulocytosis as a major, potential lethal side-effect (Capuano et al., Curr Med Chem 9: 521-548, 2002). Besides, there is still a high amount of therapy resistant cases (Lindenmayer et al., J Clin Psychiatry 63: 931-935, 2002).

In conclusion, there is still a need for developing new antipsychotics which ameliorate positive, negative and cognitive symptoms of psychosis and have a better side effect profile.

The exact pathomechanism of psychosis is not yet known. A dysfunction of several neurotransmitter systems has been shown. The two major neurotransmitter systems that are involved are the dopaminergic and the glutamatergic system:

Thus, acute psychotic symptoms may be stimulated by dopaminergic drugs (Capuano et al., Curr Med Chem 9: 521-548, 2002) and classical antipsychotics, like haloperidol, have a high affinity to the dopamine D2 receptor (Nyberg et al., Psychopharmacology 162: 37-41, 2002). Animal models based on a hyperactivity of the dopaminergic neurotransmitter system (amphetamine hyperactivity, apomorphine climbing) are used to mimic the positive symptoms of schizophrenia.

Additionally there is growing evidence that the glutamatergic neurotransmitter system plays an important role in the development of schizophrenia (Millan, Prog Neurobiol 70: 83-244, 2005). Thus, NMDA antagonists like phencyclidine and ketamine are able to stimulate schizophrenic symptoms in humans and rodents (Abi-Saab et al., Pharmacopsychiatry 31 Suppl 2: 104-109, 1998; Lahti et al., Neuropsychopharmacology 25: 455-467, 2001). Acute administration of phencyclidine and MK-801 induce hyperactivity, stereotypes and ataxia in rats mimicking psychotic symptoms. Moreover, in contrast to the dopaminergic models the animal models of psychosis based on NMDA antagonists do not only mimic the positive symptoms but also the negative and cognitive symptoms of psychosis (Abi-Saab et al., Pharmacopsychiatry 31 Suppl 2: 104-109, 1998; Jentsch and Roth, Neuropsychopharmacology 20: 201-225, 1999). Thus, NMDA antagonists, additionally induce cognitive deficits and social interaction deficits.

Eleven families of phosphodiesterases have been identified in mammals so far (Essayan, J Allergy Clin Immunol 108: 671-680, 2001). The role of PDEs in the cell signal cascade is to inactivate the cyclic nucleotides cAMP and/or cGMP (Soderling and Beavo, Proc Natl Acad USA 96(12):7071-7076, 2000). Since cAMP and cGMP are important second messenger in the signal cascade of G-protein-coupled receptors PDEs are involved in a broad range of physiological mechanisms playing a role in the homeostasis of the organism.

The PDE families differ in their substrate specificity for the cyclic nucleotides, their mechanism of regulation and their sensitivity to inhibitors. Moreover, they are differentially localized in the organism, among the cells of an organ and even within the cells. These differences lead to a differentiated involvement of the PDE families in the various physiological functions.

PDE10 (PDE10A) is primarily expressed in the brain and here in the nucleus accumbens and the caudate putamen. Areas with moderate expression are the thalamus, hippocampus, frontal cortex and olfactory tubercle (Menniti et al., William Harvey Research Conference, Porto, Dec. 6-8, 2001). All these brain areas are described to participate in the pathomechanism of schizophrenia (Lapiz et al., Neurosci Behav Physiol 33: 13-29, 2003) so that the location of the enzyme indicates a predominate role in the pathomechanism of psychosis.

In the striatum PDE10A is predominately found in the medium spiny neurons and they are primarily associated to the postsynaptic membranes of these neurons (Xie et al., Neuroscience 139: 597-607, 2006). By this location PDE10A may have an important influence on the signal cascade induced by dopaminergic and glutamatergic input on the medium spiny neurons two neurotransmitter systems playing a predominate role in the pathomechanism of psychosis.

Phosphodiesterase (PDE) 10A, in particular, hydrolyses both cAMP and cGMP having a higher affinity for cAMP (Kₘ=0.05 μM) than for cGMP (Kₘ=3 μM) (Soderling et al., Curr. Opin. Cell Biol 12: 174-179, 1999).

Psychotic patients have been shown to have a dysfunction of cGMP and cAMP levels and its downstream substrates (Kaiya, Prostaglandins Leukot Essent Fatty Acids 46: 33-38, 1992; Muly, Psychopharmacol Bull 36: 92-105, 2002; Garver et al., Life Sci 31: 1987-1992, 1982). Additionally, haloperidol treatment has been associated with increased cAMP and cGMP levels in rats and patients, respectively (Leveque et al., J Neurosci 20: 4011-4020, 2000; Gattaz et al., Biol Psychiatry 19: 1229-1235, 1984). As PDE10A hydrolyses both cAMP and cGMP (Kotera et al., Biochem Biophys Res Commun 261: 551-557, 1999), an inhibition of PDE10A would also induce an increase of cAMP and cGMP and thereby have a similar effect on cyclic nucleotide levels as haloperidol.

The antipsychotic potential of PDE 10A inhibitors is further supported by studies of Kostowski et al. (Pharm acol Biochem Behav 5: 15-17, 1976) who showed that papaverine, a moderate selective PDE10A inhibitor, reduces apomorphine-induced stereotypes in rats, an animal model of psychosis, and increases haloperidol-induced catalepsy in rats while concurrently reducing dopamine concentration in rat brain, activities that are also seen with classical antipsychotics. This is further supported by a patent application establishing papaverine as a PDE 10A inhibitor for the treatment of psychosis (US Patent Application Pub. No. 2003/0032579).

In addition to classical antipsychotics which mainly ameliorate the positive symptoms of psychosis, PDE10A also bears the potential to improve the negative and cognitive symptoms of psychosis.

Focusing on the dopaminergic input on the medium spiny neurons, PDE10A inhibitors by up-regulating cAMP and cGMP levels act as D1 agonists and D2 antagonists because the activation of Gs-protein coupled dopamine D1 receptor increases intracellular cAMP, whereas the activation of the Gi-protein coupled dopamine D2 receptor decreases intracellular cAMP levels through inhibition of adenylyl cyclase activity (Mutschler et al., Mutschler Arzneimittelwirkungen. 8^{th} ed. Stuttgart: Wissenschaftliche Verlagsgesellschaft mbH, 2001).

Elevated intracellular cAMP levels mediated by D1 receptor signalling seems to modulate a series of neuronal processes responsible for working memory in the prefrontal cortex (Sawaguchi, Parkinsonism Relat Disord 7: 9-19, 2000), and it is reported that D1 receptor activation may improve working memory deficits in schizophrenic patients (Castner et al., Science 287: 2020-2022, 2000). Thus, it seems likely that a further enhancement of this pathway might also improve the cognitive symptoms of schizophrenia.

Further indication of an effect of PDE10A inhibition on negative symptoms of psychosis was given by Rodefer et al. (Eur. J Neurosci 21: 1070-1076, 2005) who could show that papaverine reverses attentional set-shifting deficits induced by subchronic administration of phencyclidine, an NMDA antagonist, in rats. Attentional deficits including an impairment of shifting attention to novel stimuli belongs to the negative symptoms of schizophrenia. In the study the attentional deficits were induced by administering phencyclidine for 7 days followed by a washout period. The PDE10A inhibitor papaverine was able to reverse the enduring deficits induced by the subchronic treatment.

The synthesis of imidazo[1,5-a]pyrido[3,2-e]pyrazinones and some medical uses are well described in patents and the literature.

The documents EP 0 400 583 and U.S. Pat. No. 5,055,465 from Berlex Laboratories, Inc. report a group of imidazoquinoxalinones, their aza analogs and a process for their preparation. These compounds have been found to have inodilatory, vasodilatory and yenodilatory effects. The therapeutic activity is based on the inhibition of phosphodiesterase 3 (PDE3).

EP 0 736 532 reports pyrido[3,2-e]pyrazinones and a process for their preparation. These compounds are described to have anti-asthmatic and anti-allergic properties. Examples of this invention are inhibitors of PDE4 and PDE5.

WO 00/43392 reports the use of imidazo[1,5-a]pyrido[3,2-e]pyrazinones which are inhibitors of PDE3 and PDE5 for the therapy of erectile dysfunction, heart failure, pulmonic hypertonia and vascular diseases which are accompanied by insufficient blood supply.

Another group of pyrido[3,2-e]pyrazinones, reported in WO 01/68097 are inhibitors of PDE5 and can be used for the treatment of erectile dysfunction.

Further methods for the preparation of imidazo[1,5-a]pyrido[3,2-e]pyrazinones are described also by D. Norris et al. (Tetrahedron Letters 42 (2001), 4297-4299).

WO 92/22552 refers to imidazo[1,5-a]quinoxalines which are generally substituted at position 3 with a carboxylic acid group and derivatives thereof. These compounds are described to be useful as anxiolytic and sedativelhypnotic agents.

In contrast, only a limited number of imidazo[1,5-a]pyrido[3,2-e]pyrazines and their medical use are already published.

WO 99/45009 refers to a group of imidazopyrazines which are described to be inhibitors of protein tyrosine kinases used in the treatment of protein tyrosine kinase-associated disorders such as immunologic disorders. (P. Chen et al., Bioorg. Med. Chem. Lett. 12 (2002), 1361-1364 and P. Chen et al., Bioorg. Med. Chem. Lett. 12 (2002), 3153-3156).

Further PDE10 inhibitors are reported in U.S. application Ser. Nos. 11/753,207 and 11/753,260.

As is evidenced above, there is an ongoing need for improved pharmaceutical agents for the treatment of central nervous system disorders. Accordingly, the compounds and compositions provided herein are directed toward this end.

### SUMMARY

The present invention provides compounds of Formula I:

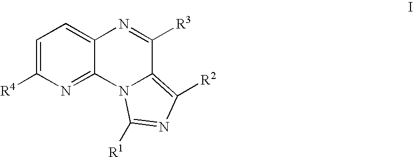

N-oxides of the same, and pharmaceutically acceptable salts thereof, wherein the variables are defined herein below.

The present invention further provides pharmaceutical compositions containing one or more of the above-described pyrido[3,2-e]pyrazine compounds of the invention, or pharmaceutically acceptable salts thereof, and at least one pharmaceutically acceptable carrier.

The present invention further provides methods of treating or preventing disorders caused by, associated with and/or accompanied by phosphodiesterase 10 hyperactivity in a patient in need thereof, the method comprising administering to said patient a therapeutically effective amount of a compound of the invention described herein, or composition thereof, or pharmaceutically acceptable salt thereof.

The present invention further provides methods of treating or preventing central nervous system disorders in a patient in need thereof, the method comprising administering to the patient a therapeutically effective amount of a compound of the invention described herein, or composition thereof, or pharmaceutically acceptable salt thereof.

The present invention further provides methods for treating or preventing obesity, type 2 diabetes, metabolic syndrome, or glucose intolerance using pyrido[3,2-e]pyrazines which are inhibitors of PDE 10. The invention further relates to methods of reducing body fat or body weight.

The present invention further provides processes for preparing the compounds of Formula (I), N-oxides of the same, or pharmaceutically acceptable salts thereof, the process comprising reacting a compound of Formula (E)

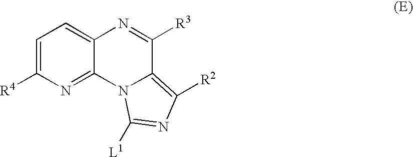

with R¹—X;

wherein the compound of Formula (E) is prepared by the process comprising reacting a compound of Formula (D)

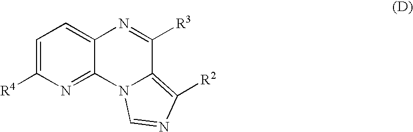

with a halogenating reagent;

wherein the compound of Formula (D) is prepared by the process comprising:

a) reacting a compound of Formula (A)

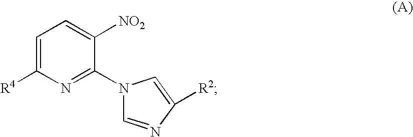

with a reducing agent to prepare a compound of Formula (B)

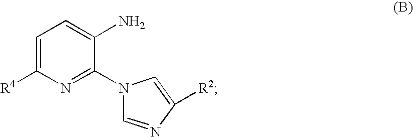

b) reacting the compound of Formula (B) with a compound of Formula:

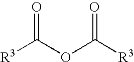

to prepare a compound of Formula (C)

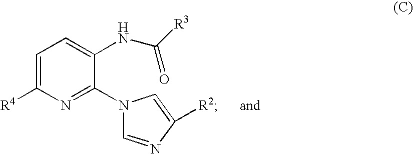

c) reacting the compound of Formula (C) with a cyclizing reagent.

Alternatively, the compound of Formula (D) can be prepared by the process comprising:

a) reacting a compound of Formula (G)

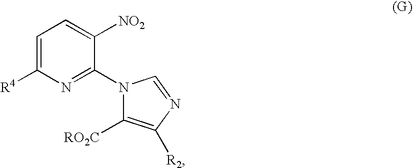

with a reducing agent to prepare a compound of Formula (H)

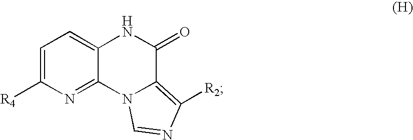

b) reacting the compound of Formula (H) with a halogenating reagent to prepare a compound of Formula (J)

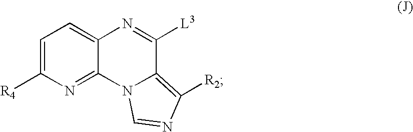

c) reacting a compound of Formula (J) with an alkylating reagent R³Y;

wherein the variables above are as defined anywhere herein.

The details of one or more embodiments of the invention are set forth in the accompanying the description below. Other features, objects, and advantages of the invention will be apparent from the description and drawings, and from the claims.

### BRIEF DESCRIPTION OF THE DRAWINGS

FIG. 1 depicts the characterization of the collected proteins from FPLC by Western blot.

FIG. 2 depicts PDE10 present in the membrane fraction.

FIG. 3 depicts the alignment of the pig PDE10 (SEQ ID NO: 5), guinea pig PDE10 (SEQ ID NO: 9), and rat PDE10 (SEQ ID NO: 10) gene sequences to provide the depicted consensus sequence (SEQ ID NO: 8).

FIG. 4 depicts the alignment of the pig PDE10 (SEQ ID NO: 11), guinea pig PDE10 (SEQ ID NO: 12), and rat PDE10 (SEQ ID NO: 13) protein sequences within the catalytic domain to provide the depicted consensus sequence (SEQ ID NO: 14).

### DETAILED DESCRIPTION

The present invention provides pyrido[3,2-e]pyrazine compounds that are PDE10 inhibitors having Formula I:

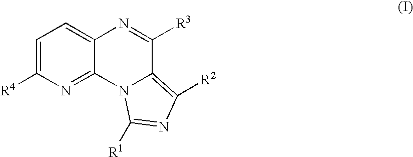

wherein:

R¹ is:

C₁₋₈ alkyl, C₂₋₈ alkenyl, C₂₋₈ alkynyl, each optionally mono- or polysubstituted with substituents independently selected from halo, OH, O—C₁₋₃ alkyl, cyano, and a cyclic radical;

aryl, heteroaryl, C₃₋₈ cyclo(hetero)alkyl, aryl-C₁₋₅ alkyl, or heteroaryl-C₁₋₅ alkyl, each optionally mono- or polysubstituted with substituents independently selected from halo, amino, C₁₋₃ alkylamino, di-C₁₋₃ alkylamino, nitro, C₁₋₅ alkyl, O—C₁₋₃ alkyl, cyano, C₁₋₃ haloalkyl, O—C₁₋₃ haloalkyl, COOH, —(C═O)—NR⁶R⁷, SO₂NR⁶R⁷, a cyclic radical, and C₃₋₈ cyclo(hetero)alkyl; or two adjacent O—C₁₋₃ alkyl groups, together with the atoms to which they are attached, form a fused 5-7 membered cycloheteroalkyl group;

R² is C₁₋₈ alkyl, C₃₋₈ cyclo(hetero)alkyl, aryl-C₁₋₅ alkyl, or heteroaryl-C₁₋₅ alkyl, each optionally mono- or polysubstituted with substituents independently selected from halo, OH, O—C₁₋₃ alkyl, and a cyclic radical;

R³ is:

cyano;

C₁₋₈ alkyl, C₁₋₈ haloalkyl, C₃₋₈ cyclo(hetero)alkyl, aryl-C₁₋₅ alkyl, heteroaryl-C₁₋₅ alkyl, each optionally mono- or polysubstituted with substituents independently selected from halo, OH, O—C₁₋₃ alkyl, and a cyclic radical;

NR⁶R⁷, (CO)OR⁶, (CO)NR⁶R⁷, NR⁵(CO)OR⁶, NR⁵(CO)R⁶, NR⁵(C═O)—NR⁶R⁷, or NR⁵(SO₂R⁶), wherein R⁵, R⁶, and R⁷ are independently selected from H, a cyclic radical, C₁₋₈ alkyl, O—C₁₋₅ alkyl, C₃₋₆ cycloalkyl, aryl-C₁₋₅ alkyl, and heteroaryl-C₁₋₅ alkyl, wherein C₁₋₈ alkyl, O—C₁₋₅ alkyl, C₃₋₆ cycloalkyl, aryl-C₁₋₅ alkyl, and heteroaryl-C₁₋₅ alkyl are optionally mono- or polysubstituted with substituents independently selected from halo, OH, O—C₁₋₃ alkyl, and a cyclic radical;

or R⁶ and R⁷, together with the nitrogen atom to which they are attached, form a 4-7 membered cycloheteroalkyl group; and

R⁴ is halo, R⁸, or OR⁸,

wherein R⁸ is:

H,

C₁₋₈ alkyl or C₃₋₆ cyclo(hetero)alkyl, each optionally mono- or polysubstituted with substituents independently selected from halo, OH, O—C₁₋₃ alkyl, C₂₋₈ alkynyl, and a cyclic radical;

aryl-C₁₋₅ alkyl or heteroaryl-C₁₋₅ alkyl, each optionally mono- or polysubstituted with substituents independently selected from halo, amino, C₁₋₃ alkylamino, di-C₁₋₃ alkylamino, nitro, C₁₋₃ alkyl, O—C₁₋₃ alkyl, and a cyclic radical;

or an N-oxide thereof, or a pharmaceutically acceptable salt thereof.

In some embodiments, R¹ is C₁₋₈ alkyl, C₂₋₈ alkenyl, or C₂₋₈ alkynyl, each optionally mono- or polysubstituted with substituents independently selected from halo and a cyclic radical.

In some embodiments, R¹ is C₁₋₈ alkyl optionally mono- or polysubstituted with halo.

In some embodiments, R¹ is propyl optionally mono- or polysubstituted with halo.

In some embodiments, R¹ is propyl optionally mono- or polysubstituted with fluoro.

In some embodiments, R¹ is C₂₋₈ alkynyl optionally mono- or polysubstituted with a cyclic radical.

In some embodiments, R¹ is C₂ alkynyl monosubstituted with a cyclic radical.

In some embodiments, R¹ is C₂ alkynyl monosubstituted with C₃₋₈ cycloalkyl.

In some embodiments, R¹ is C₂ alkynyl monosubstituted with cyclopropyl or cyclohexyl.

In some embodiments, R¹ is C₂ alkynyl monosubstituted with C₃₋₈ aryl, and said aryl is optionally mono- or polysubstituted with halo, C₁₋₃ alkyl, O—C₁₋₃ alkyl, cyano, or C₁₋₃ haloalkyl.

In some embodiments, R¹ is C₂ alkynyl monosubstituted with phenyl optionally mono- or polysubstituted with substituents independently selected from fluoro, methyl, and OCH₃.

In some embodiments, R¹ is aryl or heteroaryl each optionally mono- or polysubstituted with substituents independently selected from halo, amino, C₁₋₃ alkylamino, di-C₁₋₃ alkylamino, nitro, C₁₋₃ alkyl, O—C₁₋₃ alkyl, cyano, C₁₋₃ haloalkyl, O—C₁₋₃ haloalkyl, —(C═O)—NR⁶R⁷, and a cyclic radical.

In some embodiments, R¹ is aryl optionally mono- or polysubstituted with substituents independently selected from halo, C₁₋₃ alkyl, O—C₁₋₃ alkyl, cyano, C₁₋₃ haloalkyl, O—C₁₋₃ haloalkyl, —(C═O)—NR⁶R⁷, and a cyclic radical.

In some embodiments, R¹ is aryl mono- or polysubstituted with substituents independently selected from halo, C₁₋₃ alkyl, O—C₁₋₃ alkyl, cyano, C₁₋₃ haloalkyl, O—C₁₋₃ haloalkyl, and a cyclic radical.

In some embodiments, R¹ is aryl mono-substituted with a cyclic radical.

In some embodiments, R¹ is aryl mono-substituted with phenyl.

In some embodiments, R¹ is aryl mono-substituted with morpholino.

In some embodiments, R¹ is aryl mono-substituted with —(C═O)—NR⁶R⁷, and said R⁶ and R⁷ are independently selected from H, C₁₋₈ alkyl, and O—C₁₋₅ alkyl.

In some embodiments, R¹ is aryl mono-substituted with —(C═O)—NR⁶R⁷, and R⁶ and R⁷ are independently selected from H, methyl, and OCH₃.

In some embodiments, R¹ is aryl mono-substituted with —(C═O)—NR⁶R⁷, and said R⁶ and R⁷ together with the nitrogen atom to which they are attached, form a 4-7 membered cycloheteroalkyl group.

In some embodiments, R¹ is aryl mono-substituted with —(C═O)—NR⁶R⁷, and said R⁶ and R⁷ together with the nitrogen atom to which they are attached, form a 5-6 membered cycloheteroalkyl group.

In some embodiments, R¹ is aryl optionally mono- or polysubstituted with substituents independently selected from COOH and SO₂NR⁶R⁷.

In some embodiments, R¹ is aryl optionally mono- or polysubstituted with substituents independently selected from COOH and SO₂NH₂.

In some embodiments, R¹ is heteroaryl mono- or polysubstituted with substituents independently selected from halo, C₁₋₃ alkyl, cyano, and C₁₋₃ haloalkyl.

In some embodiments, R¹ is 5- or 6-membered heteroaryl optionally mono- or polysubstituted with substituents independently selected from halo, C₁₋₅ alkyl, amino, C₁₋₃ alkylamino, di-C₁₋₃ alkylamino, O—C₁₋₃ alkyl, cyano, C₁₋₃ haloalkyl, and a cyclic radical.

In some embodiments, R¹ is 5- or 6-membered heteroaryl optionally mono- or polysubstituted with substituents independently selected from halo, C₁₋₃ alkyl, cyano, and C₁₋₃ haloalkyl.

In some embodiments, R¹ is 5-membered heteroaryl optionally mono- or polysubstituted with substituents independently selected from amino, C₁₋₃ alkylamino, di-C₁₋₃ alkylamino, O—C₁₋₃ alkyl, and a cyclic radical.

In some embodiments R¹ is 5-membered heteroaryl optionally mono- or polysubstituted with substituents independently selected from halo, C₁₋₃ alkyl, cyano, and C₁₋₃ haloalkyl.

In some embodiments, R¹ is furan, thiophene, isoxazole, pyridine, or pyrimidine.

In some embodiments, R¹ is furan or thiophene.

In some embodiments, R¹ is pyrrole or pyrazole, each optionally mono- or polysubstituted with halo, C₁₋₃ alkyl, cyano, or C₁₋₃ haloalkyl.

In some embodiments, R¹ is pyrazole optionally mono- or polysubstituted with C₁₋₅ alkyl.

In some embodiments, R¹ is pyrazole mono-substituted with methyl.

In some embodiments, R¹ is pyrazole polysubstituted with methyl.

In some embodiments, R¹ is 1,3,5-trimethyl-1H-pyrazole-4-yl.

In some embodiments, R¹ is 3,5-dimethyl-1H-pyrazole-4-yl.

In some embodiments, R¹ is 6-membered heteroaryl optionally mono- or polysubstituted with halo, C₁₋₅ alkyl, amino, C₁₋₃ alkylamino, di-C₁₋₃ alkylamino, O—C₁₋₃ alkyl, cyano, C₁₋₃ haloalkyl, or a cyclic radical.

In some embodiments, R¹ is pyridine or pyrimidine, each optionally mono- or polysubstituted with substituents independently selected from amino, C₁₋₃ alkylamino, di-C₁₋₃ alkylamino, O—C₁₋₃ alkyl, and a cyclic radical.

In some embodiments, R¹ is pyridine or pyrimidine, each optionally mono- or polysubstituted with substituents independently selected from halo, C₁₋₅ alkyl, cyano, and C₁₋₃ haloalkyl.

In some embodiments, R¹ is pyridine optionally mono- or polysubstituted with halo or C₁₋₅ alkyl.

In some embodiments, R¹ is pyridine optionally mono- or polysubstituted with fluoro, chloro, or methyl.

In some embodiments, R¹ is pyridine mono-substituted with methyl.

In some embodiments, R¹ is 4-methylpyridin-3-yl or 2-methylpyridin-3-yl.

In some embodiments, R¹ is pyridine optionally mono-substituted with di-methylamino, OCH₃, or morpholino.

In some embodiments, R² is C₁₋₈ alkyl optionally mono- or polysubstituted with halo.

In some embodiments, R² is methyl optionally mono- or polysubstituted with halo.

In some embodiments, R² is methyl.

In some embodiments, R² is CF₃.

In some embodiments, R³ is C₁₋₈ alkyl, C₁₋₈ haloalkyl, C₃₋₈ cyclo(hetero)alkyl, aryl-C₁₋₅ alkyl, heteroaryl-C₁₋₅ alkyl, each optionally mono- or polysubstituted with substituents independently selected from halo, OH, O—C₁₋₃ alkyl, and a cyclic radical.

In some embodiments, R³ is C₁₋₈ alkyl or C₁₋₈ haloalkyl.

In some embodiments, R³ is CH₃, CH₂F, or CF₃.

In some embodiments, R³ is C₁₋₈ alkyl.

In some embodiments, R³ is C₁₋₄ alkyl.

In some embodiments, R³ is CH₃.

In some embodiments, R³ is (CO)NR⁶R⁷, and said R⁶ and R⁷ are independently selected from H or C₁₋₈ alkyl.

In some embodiments, R³ is cyano.

In some embodiments, R⁴ is OR⁸, and said R⁸ is C₁₋₈ alkyl optionally mono- or polysubstituted with substituents independently selected from halo, OH, O—C₁₋₃ alkyl, and a cyclic radical;

In some embodiments, R⁴ is OR⁸, and R⁸ is methyl optionally mono- or polysubstituted with substituents independently selected from halo, OH, O—C₁₋₃ alkyl, and a cyclic radical.

In some embodiments, R⁴ is OR⁸, and said R⁸ is C₁₋₈ alkyl optionally polysubstituted with halo.

In some embodiments, R⁸ is methyl or ethyl.

In some embodiments, R⁴ is OCH₃.

In some embodiments, R⁴ is OR⁸, and said R⁸ is C₁₋₈ alkyl optionally mono-substituted with a cyclic radical.

In some embodiments, R⁴ is OR⁸, and said R⁸ is C₁₋₈ alkyl mono- or polysubstituted with cyclopropyl.

In some embodiments, R⁴ is OR⁸, and said R⁸ is methyl mono- or polysubstituted with cyclopropyl.

In some embodiments, R⁴ is OR⁸, and said R⁸ is C₁₋₈ alkyl mono-substituted with cyclopropyl.

In some embodiments, R⁴ is OR⁸, and R⁸ is ethyl optionally mono- or polysubstituted with halo.

In some embodiments, R⁴ is OCH₂CH₂F, OCH₂CHF₂, or OCH₂CF₃.

In some embodiments, R⁴ is OR⁸, wherein said R⁸ is aryl-C₁₋₅ alkyl or heteroaryl-C₁₋₅ alkyl, each optionally mono- or polysubstituted with substituents independently selected from halo, C₁₋₃ alkyl, and O—C₁₋₃ alkyl. In some embodiments, said R⁸ is benzyl optionally mono- or polysubstituted with fluoro. In other embodiments, said R⁸ is pyridinyl.

In some embodiments:

R¹ is aryl, heteroaryl, C₃₋₈ cyclo(hetero)alkyl, aryl-C₁₋₅ alkyl, or heteroaryl-C₁₋₅ alkyl, each optionally mono- or polysubstituted with substituents independently selected from halo, amino, C₁₋₃ alkylamino, di-C₁₋₃ alkylamino, nitro, C₁₋₅ alkyl, O—C₁₋₃ alkyl, cyano, C₁₋₃ haloalkyl, O—C₁₋₃ haloalkyl, COOH, —(C═O)—NR⁶R⁷, SO₂NR⁶R⁷, and a cyclic radical; or two O—C 3 alkyl groups, together with the atoms to which they are attached, form a fused 5-7 membered cycloheteroalkyl group;

R² is C₁₋₈ alkyl;

R³ is C₁₋₈ alkyl; and

R⁴ is OR⁸, wherein R⁸ is C₁₋₈ alkyl.

In some embodiments:

R¹ is aryl or heteroaryl, each optionally mono- or polysubstituted with substituents independently selected from halo, amino, C₁₋₃ alkylamino, di-C₁₋₃ alkylamino, nitro, C₁₋₅ alkyl, O—C₁₋₃ alkyl, cyano, C₁₋₃ haloalkyl, O—C₁₋₃ haloalkyl, COOH, —(C═O)—NR⁶R⁷, SO₂NR⁶R⁷, and a cyclic radical; or two O—C₁₋₃ alkyl groups, together with the atoms to which they are attached, form a fused 5-7 membered cycloheteroalkyl group;

R² is C₁₋₈ alkyl;

R³ is C₁₋₈ alkyl; and

R⁴ is OR⁸, wherein R⁸ is C₁₋₈ alkyl.

In some embodiments:

R¹ is aryl optionally mono- or polysubstituted with substituents independently selected from halo, amino, C₁₋₃ alkylamino, di-C₁₋₃ alkylamino, nitro, C₁₋₅ alkyl, O—C₁₋₃ alkyl, cyano, C₁₋₃ haloalkyl, O—C₁₋₃ haloalkyl, COOH, —(C═O)—NR⁶R⁷, SO₂NR⁶R⁷, and a cyclic radical; or two O—C 3 alkyl groups, together with the atoms to which they are attached, form a fused 5-7 membered cycloheteroalkyl group;

R² is C₁₋₈ alkyl;

R³ is C₁₋₈ alkyl; and

R⁴ is OR⁸, wherein R⁸ is C₁₋₈ alkyl.

In some embodiments:

R¹ is heteroaryl optionally mono- or polysubstituted with substituents independently selected from halo, amino, C₁₋₃ alkylamino, di-C₁₋₃ alkylamino, nitro, C₁₋₅ alkyl, O—C₁₋₃ alkyl, cyano, C₁₋₃ haloalkyl, and O—C₁₋₃ haloalkyl;

R² is C₁₋₈ alkyl;

R³ is C₁₋₈ alkyl; and

R⁴ is OR⁸, wherein R⁸ is C₁₋₈ alkyl.

In some embodiments:

R¹ is a 5- or 6-membered heteroaryl group containing at least one ring-forming N atom, optionally mono- or polysubstituted with substituent independently selected from halo, amino, C₁₋₃ alkylamino, di-C₁₋₃ alkylamino, nitro, C₁₋₅ alkyl, O—C₁₋₃ alkyl, cyano, C₁₋₃ haloalkyl, and O—C₁₋₃ haloalkyl;

R² is C₁₋₈ alkyl;

R³ is C₁₋₈ alkyl; and

R⁴ is OR⁸, wherein R⁸ is C₁₋₈ alkyl.

In some embodiments:

R¹ is a 5- or 6-membered heteroaryl group containing at least one ring-forming N atom, optionally mono- or polysubstituted with C₁₋₅ alkyl;

R² is C₁₋₈ alkyl;

R³ is C₁₋₈ alkyl; and

R⁴ is OR⁸, wherein R⁸ is C₁₋₈ alkyl.

In some embodiments, the compounds of the invention have Formula (I):

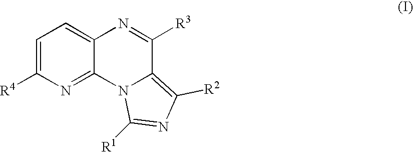

wherein:

R¹ is:

C₁₋₈ alkyl, C₂₋₈ alkenyl, C₂₋₈ alkynyl, each optionally mono- or polysubstituted with substituents independently selected from halo, cyano, and a cyclic radical;

aryl, heteroaryl, C₃₋₈ cyclo(hetero)alkyl, aryl-C₁₋₅ alkyl, or heteroaryl-C₁₋₅ alkyl, each optionally mono- or polysubstituted with substituents independently selected from halo, amino, C₁₋₃ alkylamino, di-C₁₋₃ alkylamino, nitro, C₁₋₅ alkyl, O—C₁₋₃ alkyl, cyano, C₁₋₃ haloalkyl, O—C₁₋₃ haloalkyl, COOH, —(C═O)—NR⁶R⁷, SO₂NR⁶R⁷, and cyclic radical; or two O—C₁₋₃ alkyl groups, together with the atoms to which they are attached, form a 5-7 membered cycloheteroalkyl group;

R² is C₁₋₈ alkyl optionally mono- or polysubstituted with substituents independently selected from halo and a cyclic radical;

R³ is:

cyano;

C₁₋₈ alkyl or C₁₋₈ haloalkyl each optionally mono- or polysubstituted with substituents independently selected from halo, OH, O—C₁₋₃ alkyl, and a cyclic radical;

(CO)NR⁶R⁷, wherein R⁶ and R⁷ are independently selected from H, a cyclic radical, C₁₋₈ alkyl, O—C₁₋₅ alkyl; or R⁶ and R⁷, together with the nitrogen atom to which they are attached, form a 4-7 membered cycloheteroalkyl group; and

R⁴ is R⁸ or OR⁸, wherein R⁸ is C₁₋₈ alkyl optionally mono- or polysubstituted with substituents independently selected from halo, OH, O—C₁₋₃ alkyl, C₂₋₈ alkynyl, and a cyclic radical;

or an N-oxide thereof, or a pharmaceutically acceptable salt thereof.

In some embodiments, the invention includes a compound having Formula (I):

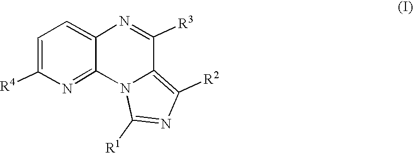

wherein:

R¹ is:

C₁₋₈ alkyl, C₂₋₈ alkenyl, C₂₋₈ alkynyl, each optionally mono- or polysubstituted with substituents independently selected from halo and a cyclic radical;

aryl, heteroaryl, C₃₋₈ cyclo(hetero)alkyl, aryl-C₁₋₅ alkyl, or heteroaryl-C₁₋₅ alkyl, each optionally mono- or polysubstituted with substituents independently selected from halo, amino, C₁₋₃ alkylamino, di-C₁₋₃ alkylamino, nitro, C₁₋₃ alkyl, O—C₁₋₃ alkyl, cyano, C₁₋₃ haloalkyl, O—C₁₋₃ haloalkyl, —(C═O)—NR⁶R⁷, and a cyclic radical; or two adjacent O—C₁₋₃ alkyl groups, together with the atoms to which they are attached, form a fused 5-7 membered cycloheteroalkyl group;

R² is C₁₋₈ alkyl optionally mono- or polysubstituted with substituents independently selected from halo and a cyclic radical;

R³ is:

cyano;

C₁₋₈ alkyl or C₁₋₈ haloalkyl each optionally mono- or polysubstituted with substituents independently selected from halo, OH, O—C₁₋₃ alkyl, and a cyclic radical; or

(CO)NR⁶R⁷, wherein R⁶ and R⁷ are independently selected from H, a cyclic radical, C₁₋₈ alkyl, O—C₁₋₅ alkyl; or R⁶ and R⁷, together with the nitrogen atom to which they are attached, form a 4-7 membered cycloheteroalkyl group;

R⁴ is R⁸ or OR⁸, wherein R⁸ is C₁₋₈ alkyl optionally mono- or polysubstituted with substituents independently selected from halo, OH, O—C₁₋₃ alkyl, and a cyclic radical;

or an N-oxide thereof, or a pharmaceutically acceptable salt thereof.

In some embodiments,

R¹ is aryl or heteroaryl, each optionally mono- or polysubstituted with substituents independently selected from halo, C₁₋₃ alkyl, and O—C₁₋₃ alkyl;

each of R² and R³ is independently C₁₋₈ alkyl; and

R⁴ is C₁₋₈ alkyl or O—C₁₋₈ alkyl.

The present invention also provides pyrido[3,2-e]pyrazine compounds that are PDE10 inhibitors having Formula I:

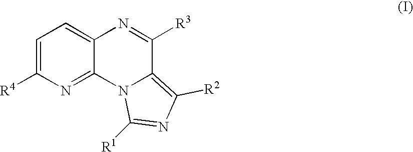

wherein:

R¹ is:

C₁₋₈ alkyl, C₂₋₈ alkenyl, or C₂₋₈ alkynyl, each optionally mono- or polysubstituted with substituents independently selected from halo, OH, O—C₁₋₃ alkyl, and a cyclic radical;

aryl, heteroaryl, C₃₋₈ cyclo(hetero)alkyl, aryl-C₁₋₅ alkyl, or heteroaryl-C₁₋₅ alkyl, each optionally mono- or polysubstituted with substituents independently selected from halo, amino, C₁₋₃ alkylamino, di-C₁₋₃ alkylamino, nitro, C₁₋₃ alkyl, O—C₁₋₃ alkyl, cyano, C₁₋₃ haloalkyl, O—C₁₋₃ haloalkyl, —(C═O)—NR⁶R⁷, and a cyclic radical; or two adjacent O—C₁₋₃ alkyl groups, together with the atoms to which they are attached, form a 5-7 membered cycloheteroalkyl group;

R² is C₁₋₈ alkyl, C₃₋₈ cyclo(hetero)alkyl, aryl-C₁₋₅ alkyl, or heteroaryl-C₁₋₅ alkyl, each optionally mono- or polysubstituted with substituents independently selected from halo, OH, O—C₁₋₃ alkyl, or a cyclic radical;

R³ is:

cyano;

C₁₋₈ alkyl, C₁₋₈ haloalkyl, C₃₋₈ cyclo(hetero)alkyl, aryl-C₁₋₅ alkyl, or heteroaryl-C₁₋₅ alkyl, each optionally mono- or polysubstituted with substituents independently selected from halo, OH, O—C₁₋₃ alkyl, or a cyclic radical;

NR⁶R⁷, (CO)OR⁶, (CO)NR⁶R⁷, NR⁵(CO)OR⁶, NR⁵(CO)R⁶, NR⁵(C═O)—NR⁶R⁷, or NR⁵(SO₂R⁶), wherein R⁵, R⁶, and R⁷ are independently selected from H, a cyclic radical, C₁₋₈ alkyl, O—C₁₋₅ alkyl, C₃₋₆ cycloalkyl, aryl-C₁₋₅ alkyl, and heteroaryl-C₁₋₅ alkyl, wherein said C₁₋₈ alkyl, O—C₁₋₅ alkyl, C₃₋₆ cycloalkyl, aryl-C₁₋₅ alkyl, and heteroaryl-C₁₋₅ alkyl are optionally mono- or polysubstituted with substituents independently selected from halo, OH, O—C₁₋₃ alkyl, or a cyclic radical;

or R⁶ and R⁷, together with the nitrogen atom to which they are attached, form a 4-7 membered cycloheteroalkyl group; and

R⁴ is halo, R⁸, or OR⁸,

wherein R⁸ is:

H,

C₁₋₈ alkyl or C₃₋₆ cyclo(hetero)alkyl, each optionally mono- or polysubstituted with substituents independently selected from halo, OH, O—C₁₋₃ alkyl, and a cyclic radical;

aryl-C₁₋₅ alkyl or heteroaryl-C₁₋₅ alkyl, each optionally mono- or polysubstituted with substituents independently selected from halo, amino, C₁₋₃ alkylamino, di-C₁₋₃ alkylamino, nitro, C₁₋₃ alkyl, O—C₁₋₃ alkyl, and a cyclic radical;

or an N-oxide thereof, or a pharmaceutically acceptable salt thereof.

In some embodiments,

R³ is:

cyano;

C₁₋₈ alkyl, C₁₋₈ haloalkyl, C₃₋₈ cyclo(hetero)alkyl, aryl-C₁₋₅ alkyl, or heteroaryl-C₁₋₅ alkyl, each optionally mono- or polysubstituted with substituents independently selected from halo, OH, O—C₁₋₃ alkyl, or a cyclic radical;

(CO)OR⁶ or (CO)NR⁶R⁷, wherein R⁵, R⁶, and R⁷ are independently selected from H, a cyclic radical, C₁₋₈ alkyl, O—C₁₋₅ alkyl, C₃₋₆ cycloalkyl, aryl-C₁₋₅ alkyl, and heteroaryl-C₁₋₅ alkyl, wherein C₁₋₈ alkyl, O—C₁₋₅ alkyl, C₃₋₆ cycloalkyl, aryl-C₁₋₅ alkyl, and heteroaryl-C₁₋₅ alkyl are optionally mono- or polysubstituted with substituents independently selected from halo, OH, O—C₁₋₃ alkyl, or a cyclic radical;

or R⁶ and R⁷, together with the nitrogen atom to which they are attached, form a 4-7 membered cycloheteroalkyl group.

The present invention further provides processes for preparing pyrido[3,2-e]pyrazine compounds that are PDE10 inhibitors, the process comprising reacting a compound of Formula (E)

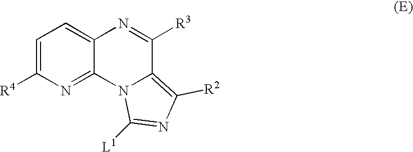

wherein L¹ is halogen;

with R¹—X, wherein X is a leaving group; to prepare said compound of Formula (I).

In some embodiments, X is B(OH)₂ or H.

In some embodiments, X is B(OH)₂. In other embodiments, X is H.

In some embodiments, the reacting is carried out in the presence of a catalyst.

In some embodiments, catalyst comprises Pd(PPh₃)₄. In other embodiments, catalyst comprises Pd(PPd₃)₂Cl₂.

In some embodiments, the reacting is carried out at an elevated temperature.

In some embodiments, the temperature is from about 85° C. to about 100° C.

In some embodiments, L¹ is bromo.

In some embodiments, the compound of Formula (E) is prepared by the process comprising reacting a compound of Formula (D):

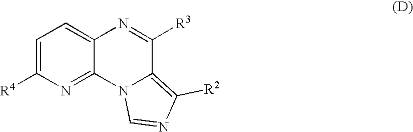

with a halogenating reagent to prepare said compound of Formula (E).

In some embodiments, the halogenating reagent is a brominating reagent. In some embodiments, brominating reagent is NBS.

In some embodiments, the compound of Formula (D) is prepared by the process comprising:

a) reacting said compound of Formula (A)

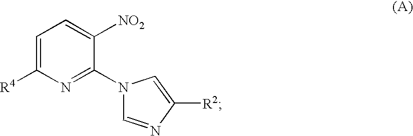

with a reducing agent to prepare a compound of Formula (B)

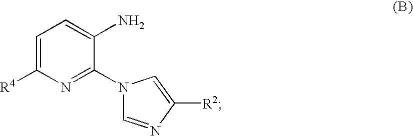

b) reacting a compound of Formula (B) with a compound of Formula:

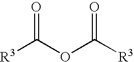

to prepare a compound of Formula (C)

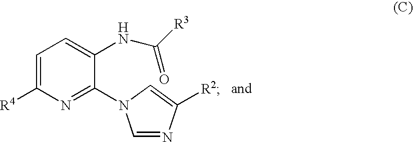

c) reacting said compound of Formula (C) with a cyclizing reagent to prepare said compound of Formula (D).

In some embodiments, R² and R³ are each C₁₋₈ alkyl and R⁴ is O—C₁₋₈ alkyl.

In some embodiments, R² is methyl, R³ is methyl, and R⁴ is methoxy.

In some embodiments, the reducing agent comprises a combination of HCO₂NH₂, 10% Pd/C, and MeOH.

In some embodiments, cyclizing reagent comprises P₂O₅/POCl₃.

In some embodiments, the compound of Formula (D) is prepared by the process comprising:

a) reacting a compound of Formula (G)

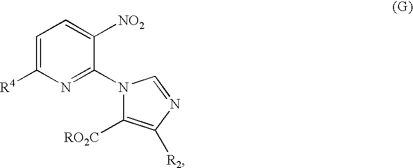

wherein R is C₁₋₄ alkyl; with a reducing agent to prepare a compound of Formula (H)

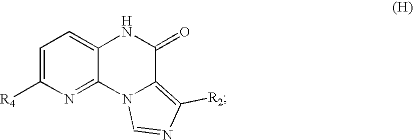

b) reacting a compound of Formula (H) with a halogenating reagent to produce a compound of Formula (J)

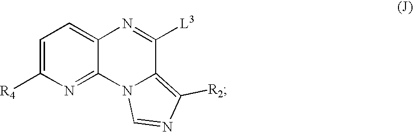

wherein L³ is halogen; and

c) reacting a compound of Formula (J) with an alkylating reagent R³Y, wherein Y is a leaving group; to prepare said compound of Formula (D).

In some embodiments, R² is C₁₋₈ haloalkyl, R³ is C₁₋₈ alkyl, and R⁴ is O—C₁₋₈ alkyl.

In some embodiments, R² is a CF₃, R³ is methyl, and R⁴ is methoxy.

In some embodiments, the reducing agent is a Na₂S₂O₄.

In some embodiments, the reacting of step (c) is carried out at an elevated temperature.

In some embodiments, the reacting of step (c) is carried out at about 90-120° C. In other embodiments, the reacting of step (c) is carried out at about 110° C.

In some embodiments, the reacting of step (c) is carried out in the presence of a catalyst.

In some embodiments, the catalyst is Pd(PPh₃)₄.

In some embodiments, R³Y is AlMe₃.

In some embodiments, the compound of Formula (D) is prepared by the process comprising reacting a compound of Formula (J)

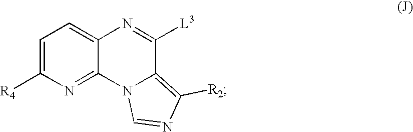

with an alkylating reagent R³Y, wherein R³ is C₁₋₈ alkyl and Y is a leaving group; to prepare said compound of Formula (D).

In some embodiments, R³ is methyl.

In some embodiments, R³Y is AlMe₃.

In some embodiments, the compound of Formula (J) is prepared by the process comprising:

a) reacting a compound of Formula (G)

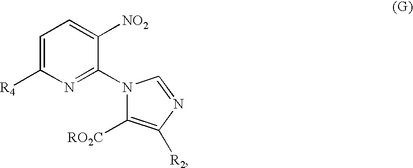

wherein R is C₁₋₄ alkyl; with a reducing agent to prepare a compound of Formula (H)

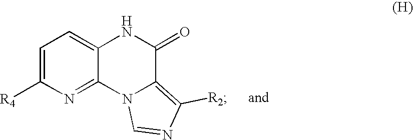

b) reacting a compound of Formula (H) with a halogenating reagent; to prepare said compound of Formula (J).

In some embodiments, R² is C₁₋₈ haloalkyl and R⁴ is O—C₁₋₈ alkyl.

In some embodiments, R² is CF₃ and R⁴ is methoxy.

In some embodiments, the reducing agent is a Na₂S₂O₄.

In some embodiments, the halogenating reagent is POCl₃.

The present invention further provides processes for preparing pyrido[3,2-e]pyrazine compounds that are PDE10 inhibitors, the process comprising:

a) reacting a compound of Formula (D):

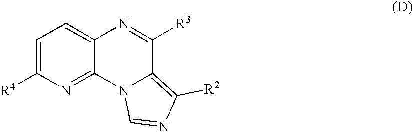

with a halogenating reagent to prepare a compound of Formula (E):

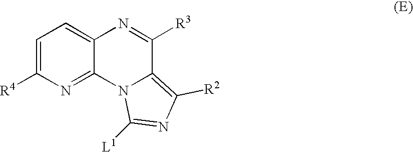

wherein L¹ is a halogen; and

b) reacting a compound of Formula (E) with R¹—X, wherein X is a leaving group; to prepare said compound of formula (I).

In some embodiments, the compound of Formula (D) is prepared by the process comprising reacting said compound of Formula (C)

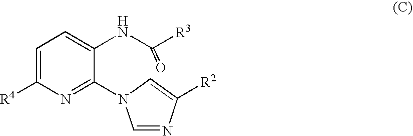

with a cyclizing reagent; to prepare said compound of Formula (D).

In some embodiments, the compound of Formula (C) is prepared by the process comprising:

a) reacting a compound of Formula (A)

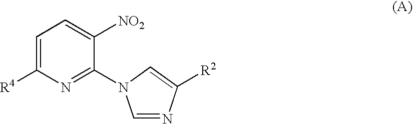

with a reducing agent to prepare a compound of Formula (B)

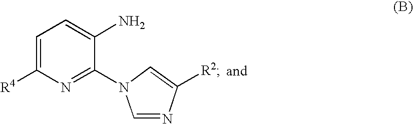

b) reacting a compound of Formula (B) with a compound of Formula:

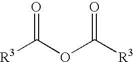

to prepare said compound of Formula (C).

In some embodiments, the compound of Formula (D) is prepared by the process comprising:

a) reacting a compound of Formula (G)

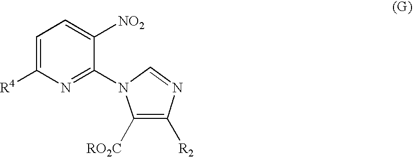

wherein R is C₁₋₄ alkyl; with a reducing agent to prepare a compound of Formula (H)

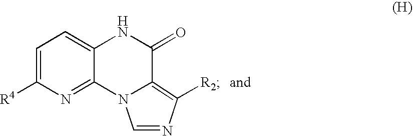

b) reacting a compound of Formula (H) with a halogenating reagent to produce said compound of Formula (J)

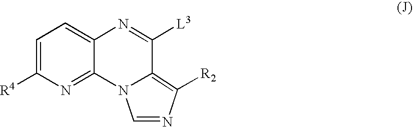

wherein L³ is halogen; and

c) reacting a compound of Formula (J) with an alkylating reagent R³Y, wherein Y is a leaving group; to prepare said compound of Formula (D).

In some embodiments, the compound of Formula (D) is prepared by the process comprising reacting a compound of Formula (J)

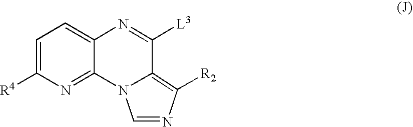

with an alkylating reagent R³Y, wherein R³ is C₁₋₈ alkyl and Y is a leaving group.

In some embodiments, the compound of Formula (J) is prepared by the process comprising:

a) reacting a compound of Formula (G)

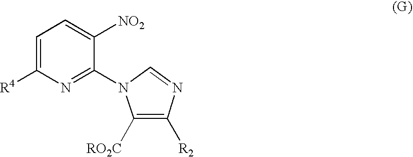

wherein R is C₁₋₄ alkyl; with a reducing agent to prepare a compound of Formula (H)

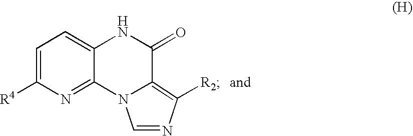

b) reacting a compound of Formula (H) with a halogenating reagent; to prepare said compound of Formula (J).

The present invention further provides processes for preparing pyrido[3,2-e]pyrazine compounds that are PDE10 inhibitors, the process comprising:

a) reacting a compound of Formula (J)

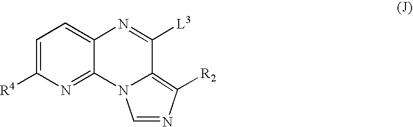

wherein L³ is halogen;

with an alkylating reagent R³Y to prepare a compound of Formula (D)

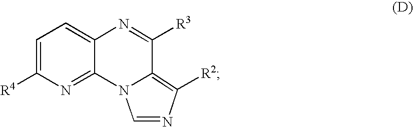

b) reacting a compound of Formula (D) with a halogenating reagent to prepare a compound of Formula (E):

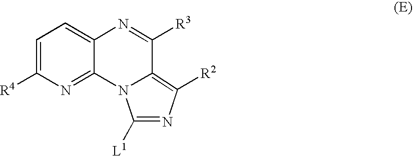

wherein L¹ is a halogen; and

b) reacting a compound of Formula (E) with R¹—X, wherein X is a leaving group; to prepare said compound of Formula (I).

In some embodiments, the compound of Formula (J) is prepared by the process comprising:

c) reacting a compound of Formula (G)

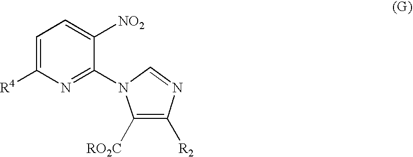

wherein R is C₁₋₄ alkyl; with a reducing agent to prepare a compound of Formula (H)

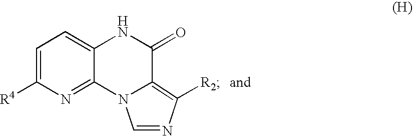

d) reacting a compound of Formula (H) with a halogenating reagent; to prepare said compound of Formula (J).

The present invention further provides processes for preparing pyrido[3,2-e]pyrazine compounds that are PDE 10 inhibitors. Example processes are provided below in Schemes 1 and 2,

wherein the variables are independently defined anywhere herein.

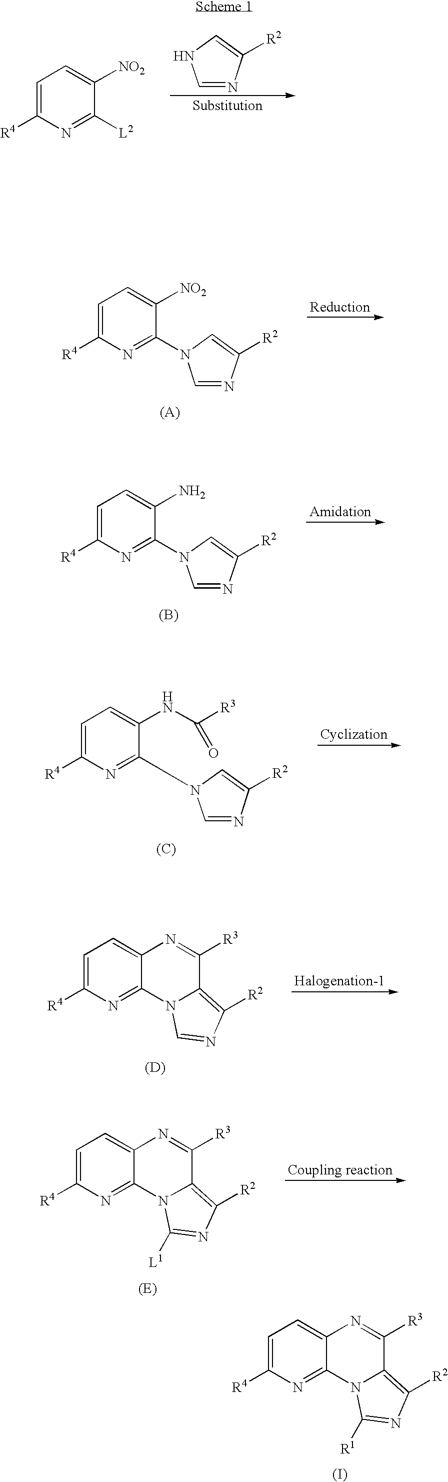

In one aspect of the invention are provided processes, such as are exemplified by Scheme 1, that involves compounds of Formulas (I), (F), (G), (H), (J), (D), and (E), or salt forms of the compounds.

### Coupling Reaction

The compounds of Formula (I) can be prepared via a coupling reaction affixing the R¹ substituent to the imidazole portion of the ring as a final step. Example processes of the invention include Suzuki and Sonogashira methods using aryl derivatives or alkynyl derivatives, respectively.

Accordingly, the compounds of Formula (I) can be prepared by reacting a compound of Formula (E)

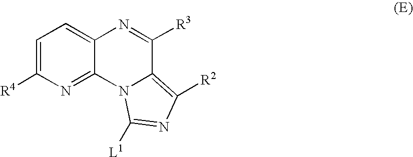

wherein L¹ is a leaving group;

with R¹—X, wherein X is a leaving group; to prepare a compound of Formula (I).

In some embodiments, X is B(OH)₂ or H. In some embodiments, X is B(OH)₂. In some embodiments, X is H.

In some embodiments, the coupling reaction can be carried out at an elevated temperature, e.g., at about 40-100° C., about 50-100° C., about 60-100° C., about 70-100° C., about 80-100° C., about 85-100° C., or about 85-90° C., or about 90-100° C., or about 85° C., or about 90° C. The coupling reaction can also be carried out in the presence of water. In some embodiments, the molar ratio of water to organic solvent is about 1:2, about 1:3, or about 1:4. Suitable organic solvents include, DMF, dioxane, THF, or acetonitrile. In some embodiments, the coupling reaction employs either an organic base or an inorganic base. Suitable organic bases include, but are not limited to, triethylamine, diisopropylethylamine, and pyridine. Suitable inorganic bases include, but are not limited to, NaOH and K₂CO₃. In some embodiments, the leaving group L¹ can be chloro, bromo, or iodo. In other embodiments, the leaving group L¹ can be bromo. In some embodiments, R¹ is optionally substituted aryl or heteroaryl. In some embodiments, R¹ is alkyl substituted with aryl or heteroaryl. In some embodiments, the coupling reaction can be carried out in the presence of a catalyst. In some embodiments, the catalyst is a palladium catalyst such as Pd(PPh₃)₂Cl₂ or Pd(PPh₃)₄. In some embodiments, the catalyst further comprises CuI. In some embodiments, the coupling reaction is the Suzuki coupling reaction (See, e.g., Suzuki, A. Pure & Appl. Chem. 1985, 57, 1749). In some embodiments, the coupling reaction is the Sonogashira coupling reaction (See (a) Sonogashira, Comprehensive Organic Synthesis, Volume 3, Chapters 2, 4; (b) Sonogashira, Synthesis 1977, 777).

### Halogenation Reaction

According to a further aspect of the invention, a compound of Formula (E) can be prepared by reacting a compound of Formula (D):

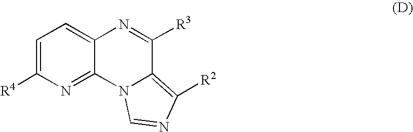

with a halogenating reagent.

Any of numerous halogenating reagents known in the art can be used. In some embodiments, the halogenating reagent is a brominating or chlorinating reagent. Some example brominating reagents include, for example, Br₂, N-bromosuccinimide (NBS), 1,3-dibromo-5,5-dimethylhydantoin, pyridinium tribromide (pyrHBr₃) and the like. An example chlorinating reagent is N-chlorosuccinimide. In some embodiments, the halogenating reagent is N-bromosuccinimide.

Any suitable organic solvent can be optionally used to carry out the halogenating reaction. In some embodiments, the organic solvent contains an alcohol such as methanol, ethanol, n-propanol, isopropanol, butanol, mixtures thereof and the like. In some embodiments, the organic solvent is acetonitrile. In some embodiments, the organic solvent is methanol. In further embodiments, the organic solvent includes dimethylformamide or tetrahydrofuran. Suitable temperatures for the halogenating reaction can vary. For example, the reaction temperature can be at or below about room temperature such as, for example, from about 0 to about 25° C. The molar ratio of halogenating reagent to compound of Formula (D) can be routinely selected or optimized by the skilled artisan to minimize di-halogenated by products and maximize yield of the mono-halogenated product. In some embodiments, the molar ratio is from about 1:0.8 to about 1:1:2, from about 1:0.9 to about 1:1.1, from about 1:0.95 to about 1:1.05, or about 1:1.

### Cyclization Reaction

According to a further aspect, a compound of Formula (D) can be prepared by reacting a compound of Formula (C)

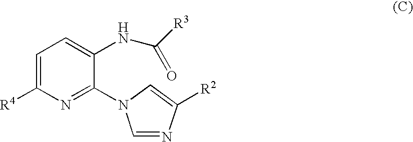

with a cyclizing reagent to prepare said compound of Formula (D).

Suitable cyclizing reagents include, but are not limited to, POCl₃, PCl₅, P₂O₅, or SOCl₂. In some embodiments, the cyclizing reagent comprises P₂O₅/POCl₃. In some embodiments, the cyclizing reagent can be a combination of two reagents, e.g., P₂O₅/POCl₃. In some embodiments, the cyclization reaction is carried out in the presence of a base, e.g., an organic base such as triethylamine, diisopropylamine, or pyridine. In some embodiments, the cyclization reaction is carried out at an elevated temperature, such as about 90-120° C., about 100-120° C., or about 110-120° C. In some embodiments, the cyclization reaction is carried out for a certain time, such as about 2-6 hours, or about 4-6 hours, or about 6 hours. In some embodiments, the cyclizing reaction is carried out under anhydrous conditions.

### Amidation Reaction

According to a further aspect of the invention, the compound of Formula (C) can be prepared by reacting a compound of Formula (B)

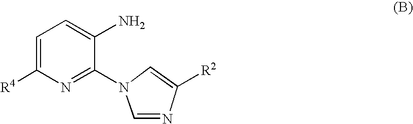

with a compound of Formula:

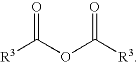

In some embodiments, the reaction can be carried out at room temperature. In some embodiments, the reaction can be carried out at an elevated temperature, e.g., 40-80° C., 50-80° C., 60-80° C., or 70-80° C. In some embodiments, the reaction solvent comprises toluene (e.g., toluene or a mixture of toluene and heptane).

In some embodiments, R² and R³ are each C₁₋₈ alkyl and R⁴ is O—C₁₋₈ alkyl.

In some embodiments, R² is methyl, R³ is ethyl, and R⁴ is methoxy. In other embodiments, R² is methyl, R³ is methyl, and R⁴ is methoxy.

### Reduction Reaction

According to a further aspect, a compound of Formula (B) can be prepared by reacting a compound of Formula (A):

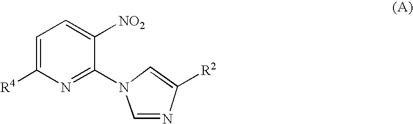

with a reducing agent.

The nitro group of a compound of Formula (A) can be reduced to the corresponding amino group by numerous reducing agents known in the art including, but not limited to, hydrogen (usually in the presence of a metal catalyst such as Pd), tin chloride, Na₂S₂O₄, or a combination of 10% Pd—C/HCO₂NH₄/CH₃OH. In some embodiments, the reducing agent is tin chloride. In some embodiments, the reducing agent comprises a combination of HCO₂NH₄, 10% Pd/C, and MeOH. In some embodiments, the reaction is carried out at room temperature. In some embodiments, the reduction reaction is carried out at an elevated temperature, e.g., about 35-60° C., about 45-60° C., about 50-60° C., or about 55-60° C.

### Substitution Reaction

According to a further aspect, a compound of Formula (A) can be prepared by reacting a compound of Formula:

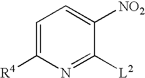

wherein L² is a leaving group;

with a compound of Formula:

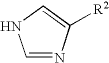

to prepare a compound of Formula (A).

The substitution reaction can be carried out in the presence of a base. In some embodiments, the base can be sodium hydroxide, potassium hydroxide, sodium carbonate, cesium carbonate, or potassium carbonate. In some embodiments, the base such as sodium hydroxide or potassium hydroxide can be used in a powder form. Suitable solvents for the substitution reaction include, but are not limited to, polar or weakly polar solvents such as DMF, THF, DMSO, NMP, or dioxane.

In some embodiments, the leaving group L² is halo, for example, bromo, chloro, or fluoro. In some embodiments, L² is chloro.

In another aspect of the invention are provided processes, such as are exemplified by Scheme 2, that involves compounds of Formulas (I), (F), (G), (H), (J), (D), and (E), or salt forms of the compounds.

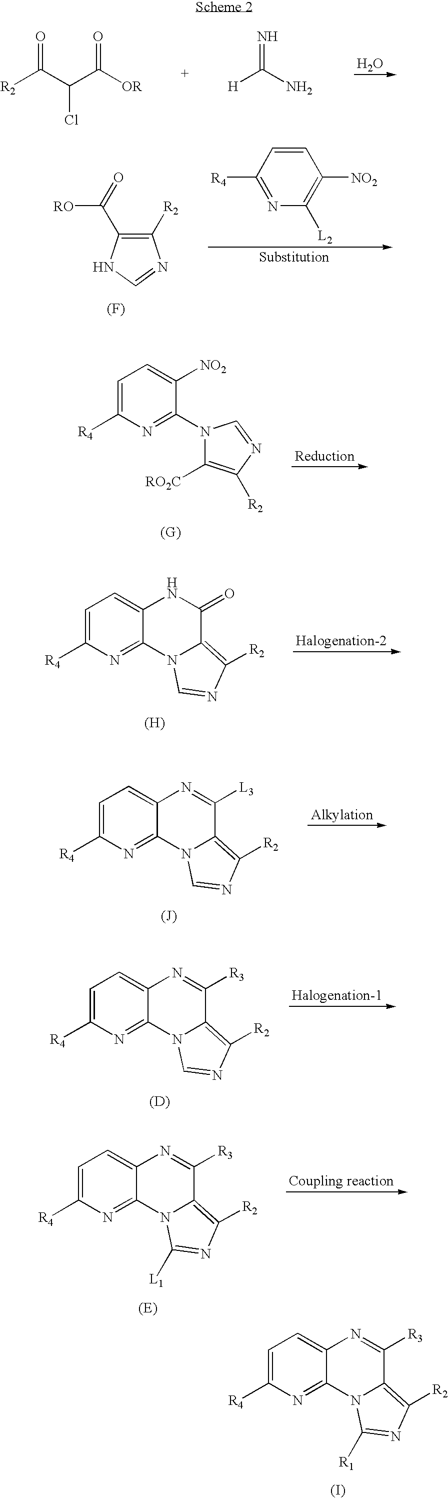

### Coupling Reaction and Halogenation Reaction

The coupling reaction and the Halogenation reaction (Halogenation-1) in Scheme 2 can be carried out as in Scheme 1.

### Alkylation Reaction

According to a further aspect of the invention, a compound of Formula (D) can be prepared by the process comprising reacting a compound of Formula (J)

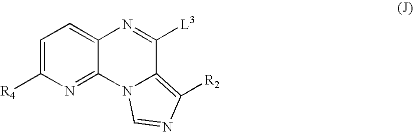

wherein L³ is halogen;

with an alkylating reagent R³Y, wherein Y is a leaving group; to prepare the compound of Formula (D).

The alkylation reaction can be carried out at an elevated temperature. In some embodiments, the temperature can be about 70-120° C., about 80-120° C., about 90-120° C., about 100-120° C., about 105-120° C., about 110-120° C., about 110° C., or about 120° C. Suitable solvents include, but are not limited to, DMF, N-methyl-2-pyrrolidinone, toluene, or dioxane. The alkylating agents R³Y can include alkyl halides or other alkylating agents such as organometallic compounds, e.g., Grinard reagents, organolithium reagents, organocopper reagents, or organoaluminum reagents. In some embodiments, the alkylating agents R³Y is a Grinard reagent. In some embodiments, the alkylating agent R³Y is an organoaluminum reagent. In some embodiments, the alkylating agent R³Y is trimethylaluminum. In some embodiments, the alkylation reaction can be carried out in the presence of a catalyst. In some embodiments, the alkylation reaction can be catalyzed by a palladium catalyst, for example, Pd(PPh₃)₄.

In some embodiments, R² is C₁₋₈ haloalkyl, R³ is C₁₋₈ alkyl, and R⁴ is O—C₁₋₈ alkyl.

In some embodiments, R² is a CF₃, R³ is methyl, and R⁴ is methoxy.

In some embodiments, R is ethyl.

### Halogenation Reaction-2

According to a further aspect of the invention, a compound of Formula (J) can be prepared by reacting a compound of Formula (H)

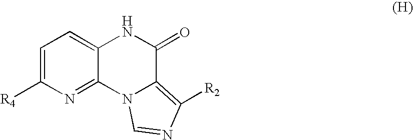

with a halogenating reagent to produce the compound of Formula (J).

In some embodiments, the halogenation reaction requires an organic solvent. In some embodiments, the halogenation reaction is a neat reaction (i.e., substantially no solvent is required). In some embodiments, the halogenating reagent can be POCl₃, PCl₃, SOCl₂, or PPh₃/CCl₄. In some embodiments, the halogenating reagent is POCl₃. The halogenation reaction temperature can be about 60-130° C., about 70-130° C., about 80-130° C., about 90-130° C., about 100-130° C., about 110-130° C., or about 120-130° C.

### Reduction/Cyclization Reaction

According to a further aspect of the invention, a compound of Formula (H) can be prepared by reacting a compound of Formula (G):

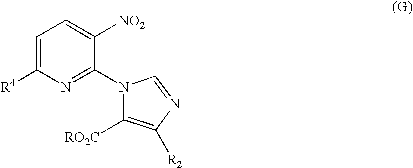

with a reducing agent to prepare the compound of Formula (H).

The reduction reaction can be carried out by numerous reducing agents known in the art. Example reducing agents include, but not limited to, catalystic hydrogenation, tin chloride, Na₂S₂O₄, or a combination of 10% Pd—C/HCO₂NH₄/CH₃OH. In some embodiments, the reducing agent comprises tin chloride. In some embodiments, the reducing agent comprises Na₂S₂O₄. Any suitable solvent can be optionally used to carry out the reduction reaction. The solvent can include organic solvents or inorganic solvents. In some embodiments, the solvent is a mixture of two or more solvents. In some embodiments, the solvent is anhydrous. In some embodiments, the solvent comprises water. In some embodiments, the solvent is a mixture of water and an organic solvent. The organic solvent can be fully miscible with water. For example, the solvent can be an alcohol (e.g., methanol or ethanol), THF, or acetic acid. In some embodiments, the solvent is a mixture of water and acetic acid. The molar ratio of water and acetic acid can be about 1:1.5, about 1:1.6, about 1:1.7, about 1:1.8, about 1:1.9, or about 1:2.0. The reduction reaction can be carried out at an elevated temperature, e.g., about 70-110° C., about 80-110° C., about 90-110° C., or about 100-110° C.

In some embodiments, R² is C₁₋₈ haloalkyl and R⁴ is O—C₁₋₈ alkyl. In other embodiments, R² is CF₃ and R⁴ is methoxy.

In some embodiments, R is ethyl.

### Substitution Reaction

According to a further aspect of the invention, a compound of Formula (G) can be prepared by reacting a compound of Formula (F):

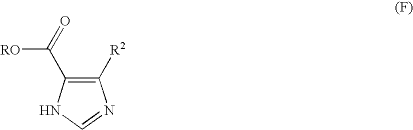

with a compound of Formula:

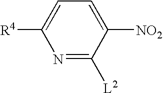

wherein L² is a leaving group;

to prepare the compound of Formula (G).

The substitution reaction can be carried out in the same way as provided in Scheme 1.

### Imidazole Formation

According to a further aspect of the invention, a compound of Formula (F) can be prepared by reacting a compound of Formula:

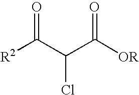

wherein R is C₁₋₄ alkyl;

with HC(═NH)NH₂ to prepare the compound of Formula (F).

The imidazole formation reaction can be carried out at an elevated temperature, e.g., about 60-140° C., about 80-140° C., about 100-140° C., about 110-140° C., or about 120-140° C. In some embodiments, the imidazole formation reaction can be carried out in a polar protic solvent. Example polar protic solvents include, but are not limited to, water, methanol, and acetic acid.

### DEFINITIONS

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further intended that the compounds of the invention are stable. As used herein “stable” refers to a compound that is sufficiently robust to survive isolation to a useful degree of purity from a reaction mixture, and preferably capable of formulation into an efficacious therapeutic agent.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 10, from 1 to about 8, from 1 to about 6, from 1 to about 4, or from 1 to about 3 carbon atoms.

As used herein, “alkenyl” refers to an alkyl group having one or more double carbon-carbon bonds. Example alkenyl groups include ethenyl, propenyl, and the like.

As used herein, “alkynyl” refers to an alkyl group having one or more triple carbon-carbon bonds. Example alkynyl groups include ethynyl, propynyl, and the like.

As used herein, “haloalkyl” refers to an alkyl group having one or more halogen substituents. Example haloalkyl groups include CF₃, C₂F₅, CHF₂, CCl₃, CHCl₂, C₂Cl₅, and the like.

As used herein, “cyclic radical” refers to a saturated, unsaturated, or aromatic carbocycle or heterocycle, optionally mono- or polysubstituted with halo, amino, C₁₋₃ alkylamino, di-C₁₋₃ alkylamino, nitro, C₁₋₃ alkyl, OH, or O—C₁₋₃ alkyl. The cyclic radical can be a 3 to 24 membered mono- or polycyclic ring. In some embodiments, the cyclic radical is a 3-, 4-, 5-, 6-, or 7-membered ring. The cyclic radical can contain 3 to 20, or in some embodiments, 4 to 10 ring forming carbon atoms. The cyclic radical includes cyclo(hetero)alkyl, aryl and heteroaryl groups as defined below. “Cyclo(hetero)alkyl” refers to both cycloalkyl and cycloheteroalkyl groups. Cycloheteroalkyl and heteroaryl groups may, for example, contain 1 to 6, or in some embodiments, 1 to 3 ring forming heteroatoms, selected from O, N, S, and/or P. The cyclic radical can be bound via a carbon atom or optionally via a N, O, S, SO, or SO₂ group. An example of an aryl cyclic radical is phenyl. Examples of cycloalkyl cyclic radicals include cyclopropyl, cyclobutyl, cyclopentyl, cyclohexyl, and cycloheptyl. Examples of heteroaryl cyclic radicals include thienyl, furanyl, pyrroyl, imidazolyl, triazolyl, oxazolyl, isoxazoyl, pyrazolyl, thiazolyl, pyridinyl, pyrimidinyl, and the like. Examples of cycloheteroalkyl cyclic radicals include pyrrolidinyl, tetrahydrofuranyl, morpholino, thiomorpholino, piperazinyl, tetrahydrothienyl, 2,3-dihydrobenzofuryl, 1,3-benzodioxole, benzo-1,4-dioxane, piperidinyl, isoxazolidinyl, isothiazolidinyl, pyrazolidinyl, oxazolidinyl, thiazolidinyl, and imidazolidinyl. Examples of heteroaryl groups are provided below.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, and the like. In some embodiments, an aryl group has from 6 to about 20 carbon atoms.

As used herein, “arylalkyl” refers to an alkyl group substituted by an aryl group. Example arylalkyl groups include benzyl and phenylethyl.

As used herein, “cycloalkyl” refers to non-aromatic carbocycles including cyclized alkyl, alkenyl, and alkynyl groups. Cycloalkyl groups can include mono- or polycyclic (e.g., having 2, 3 or 4 fused rings) ring systems, including spirocycles. In some embodiments, cycloalkyl groups can have from 3 to about 20 carbon atoms, 3 to about 14 carbon atoms, 3 to about 10 carbon atoms, or 3 to 7 carbon atoms. Cycloalkyl groups can further have 0, 1, 2, or 3 double bonds and/or 0, 1, or 2 triple bonds. Also included in the definition of cycloalkyl are moieties that have one or more aromatic rings fused (i.e., having a bond in common with) to the cycloalkyl ring, for example, benzo derivatives of cyclopentane, cyclopentene, cyclohexane, and the like. A cycloalkyl group having one or more fused aromatic rings can be attached through either the aromatic or non-aromatic portion. One or more ring-forming carbon atoms of a cycloalkyl group can be oxidized, for example, having an oxo or sulfido substituent. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcamyl, adamantyl, and the like.

As used herein, a “heteroaryl” group refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Any ring-forming N atom in a heteroaryl group can also be oxidized to form an N-oxo moiety. Examples of heteroaryl groups include without limitation, pyridyl, N-oxopyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, a “heteroarylalkyl” group refers to an alkyl group substituted by a heteroaryl group. An example of a heteroarylalkyl group is pyridylmethyl.

As used herein, “cycloheteroalkyl” refers to a non-aromatic heterocycle where one or more of the ring-forming atoms is a heteroatom such as an O, N, or S atom. Cycloheteroalkyl groups can include mono- or polycyclic (e.g., having 2, 3 or 4 fused rings) ring systems as well as spirocycles. Example cycloheteroalkyl groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Also included in the definition of cycloheteroalkyl are moieties that have one or more aromatic rings fused (i.e., having a bond in common with) to the nonaromatic heterocyclic ring, for example phthalimidyl, naphthalimidyl, and benzo derivatives of heterocycles. A cycloheteroalkyl group having one or more fused aromatic rings can be attached though either the aromatic or non-aromatic portion. Also included in the definition of cycloheteroalkyl are moieties where one or more ring-forming atoms is substituted by 1 or 2 oxo or sulfido groups. In some embodiments, the cycloheteroalkyl group has from 1 to about 20 carbon atoms, and in further embodiments from about 3 to about 20 carbon atoms. In some embodiments, the cycloheteroalkyl group contains 3 to about 20, 3 to about 14, 3 to about 7, or 5 to 6 ring-forming atoms. In some embodiments, the cycloheteroalkyl group has 1 to about 4, 1 to about 3, or 1 to 2 heteroatoms. In some embodiments, the cycloheteroalkyl group contains 0 to 3 double bonds. In some embodiments, the cycloheteroalkyl group contains 0 to 2 triple bonds.

As used herein, “halo” or “halogen” includes fluoro, chloro, bromo, and iodo.

As used herein, “haloalkyl” refers to an alkyl group substituted by one or more halogen atoms. Examples of haloalkyl groups include CF₃ and CF₂CF₃.

As used herein, “alkoxy” refers to an —O-alkyl group. Example alkoxy groups include methoxy, ethoxy, propoxy (e.g., n-propoxy and isopropoxy), t-butoxy, and the like.

As used herein, the term “substituted” refers to the replacement of a hydrogen moiety with a non-hydrogen moiety in a molecule or group. The term “polysubstituted” means substituted with more than one substituent up to the valence of the substituted group. For example, a polysubstituted group can be substituted with 2, 3, 4, or 5 substituents. Generally when a list of possible substituents is provided, the substituents can be independently selected from that group.

As used herein, the term “leaving group” refers to a moiety that can be displaced by another moiety, such as by nucleophilic attack, during a chemical reaction. Leaving groups are well known in the art and include, for example, halogen, hydroxy, alkoxy, —O(CO)R^{a}, —OSO₂—R^{b}, and —Si(R^{c})₃ wherein R^{a} can be C₁₋₈ alkyl, C₃₋₇ cycloalkyl, aryl, heteroaryl, or cycloheteroalkyl, wherein R^{b} can be C₁₋₈ alkyl, aryl (optionally substituted by one or more halo, cyano, nitro, C₁₋₄ alkyl, C₁₋₄ haloalkyl, C₁₋₄ alkoxy, or C₁₋₄ haloalkoxy), or heteroaryl (optionally substituted by one or, more halo, cyano, nitro, C₁₋₄ alkyl, C₁₋₄ haloalkyl, C₁₋₄ alkoxy, or C₁₋₄ haloalkoxy), and wherein R^{c} can be C₁₋₈ alkyl. Example leaving groups include chloro, bromo, iodo, mesylate, tosylate, trimethylsilyl, and the like.

The term “reacting” is meant to refer to the bringing together of the indicated reagents in such a way as to allow their molecular interaction and chemical transformation according to the thermodynamica and kinetics of the chemical system. Reacting can be facilitated, particularly for solid reagents, by using an appropriate solvent or mixture of solvents in which at least one of the reagents is at least partially soluble. Reacting is typically carried out for a suitable time and under conditions suitable to bring about the desired chemical transformation.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

In the case of the compounds which contain an asymmetric carbon atom, the invention relates to the D form, the L form, and D,L mixtures and also, where more than one asymmetric carbon atom is present, to the diastereomeric forms. Those compounds of the invention which contain asymmetric carbon atoms, and which as a rule accrue as racemates, can be separated into the optically active isomers in a known manner, for example using an optically active acid. However, it is also possible to use an optically active starting substance from the outset, with a corresponding optically active or diastereomeric compound then being obtained as the end product.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the invention can also include all isotopes of atoms occurring in the intermediates or final compounds. Isotopes include those atoms having the same atomic number but different mass numbers. For example, isotopes of hydrogen include tritium and deuterium.

The term, “compound,” as used herein is meant to include all stereoisomers, geometric isomers, tautomers, and isotopes of the structures depicted.

All compounds, and pharmaceutically acceptable salts thereof, are also meant to include solvated or hydrated forms.

In some embodiments, the compounds of the invention, and salts thereof, are substantially isolated. By “substantially isolated” is meant that the compound is at least partially or substantially separated from the environment in which it was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof. Methods for isolating compounds and their salts are routine in the art.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

The physiologically acceptable salts may be obtained by neutralizing the bases with inorganic or organic acids or by neutralizing the acids with inorganic or organic bases. Examples of suitable inorganic acids are hydrochloric acid, sulphuric acid, phosphoric acid, or hydrobromic acid, while examples of suitable organic acids are carboxylic acid, sulpho acid, or sulphonic acid, such as acetic acid, tartaric acid, lactic acid, propionic acid, glycolic acid, malonic acid, maleic acid, fumaric acid, tannic acid, succinic acid, alginic acid, benzoic acid, 2-phenoxybenzoic acid, 2-acetoxybenzoic acid, cinnamic acid, mandelic acid, citric acid, maleic acid, salicylic acid, 3-aminosalicylic acid, ascorbic acid, embonic acid, nicotinic acid, isonicotinic acid, oxalic acid, gluconic acid, amino acids, methanesulphonic acid, ethanesulphonic acid, 2-hydroxyethanesulphonic acid, ethane-1,2-disulphonic acid, benzenesulphonic acid, 4-methylbenzenesulphonic acid or naphthalene-2-sulphonic acid. Examples of suitable inorganic bases are sodium hydroxide, potassium hydroxide and ammonia, while examples of suitable organic bases are amines, e.g., tertiary amines, such as trimethylamine, triethylamine, pyridine, N,N-dimethylaniline, quinoline, isoquinoline, α-picoline, β-picoline, γ-picoline, quinaldine, or pyrimidine.

In addition, physiologically acceptable salts of the compounds according to formula (I) can be obtained by converting derivatives which possess tertiary amino groups into the corresponding quaternary ammonium salts in a manner known per se using quaternizing agents. Examples of suitable quaternizing agents are alkyl halides, such as methyl iodide, ethyl bromide, and n-propyl chloride, and also arylalkyl halides, such as benzyl chloride or 2-phenylethyl bromide.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

### Compositions and Administration

The compounds of the present invention are inhibitors of phosphodiesterase 10. It is therefore a part of the subject-matter of this invention that the compounds according to formula (I), and their salts and also pharmaceutical preparations which comprise these compounds or their salts, can be used for treating or preventing disorders caused by, associated with and/or accompanied by phosphodiesterase 10 hyperactivity and/or disorders in which inhibiting phosphodiesterase 10 is of value. It is an embodiment of this invention, that compounds of formula (I) including their salts, solvates and prodrugs and also pharmaceutical compositions comprising an amount of a compound of formula (I) or one of its salts, solvates or prodrugs effective in inhibiting PDE10 can be used for the treatment of central nervous system disorders of mammals including a human.

An effective dose of the compounds according to the invention, or their salts, is used, in addition to physiologically acceptable carriers, diluents and/or adjuvants for producing a pharmaceutical composition. The dose of the active compounds can vary depending on the route of administration, the age and weight of the patient, the nature and severity of the diseases to be treated, and similar factors. The daily dose can be given as a single dose, which is to be administered once, or be subdivided into two or more daily doses, and is as a rule 0.001-2000 mg. Particular preference is given to administering daily doses of 0.1-500 mg, e.g. 0.1-100 mg.

Suitable administration forms are oral, parenteral, intravenous, transdermal, topical, inhalative, intranasal and sublingual preparations. Particular preference is given to using oral, parenteral, e.g. intravenous or intramuscular, intranasal, e.g. dry powder or sublingual preparations of the compounds according to the invention. The customary galenic preparation forms, such as tablets, sugar-coated tablets, capsules, dispersible powders, granulates, aqueous solutions, alcohol-containing aqueous solutions, aqueous or oily suspensions, syrups, juices or drops, are used.

Solid medicinal forms can comprise inert components and carrier substances, such as calcium carbonate, calcium phosphate, sodium phosphate, lactose, starch, mannitol, alginates, gelatine, guar gum, magnesium stearate, aluminium stearate, methyl cellulose, talc, highly dispersed silicic acids, silicone oil, higher molecular weight fatty acids, (such as stearic acid), gelatine, agar agar or vegetable or animal fats and oils, or solid high molecular weight polymers (such as polyethylene glycol); preparations which are suitable for oral administration can comprise additional flavourings and/or sweetening agents, if desired.

Liquid medicinal forms can be sterilized and/or, where appropriate, comprise auxiliary substances, such as preservatives, stabilizers, wetting agents, penetrating agents, emulsifiers, spreading agents, solubilizers, salts, sugars or sugar alcohols for regulating the osmotic pressure or for buffering, and/or viscosity regulators.

Examples of such additives are tartrate and citrate buffers, ethanol and sequestering agents (such as ethylenediaminetetraacetic acid and its nontoxic salts). High molecular weight polymers, such as liquid polyethylene oxides, microcrystalline celluloses, carboxymethyl celluloses, polyvinylpyrrolidones, dextrans or gelatine, are suitable for regulating the viscosity. Examples of solid carrier substances are starch, lactose, mannitol, methyl cellulose, talc, highly dispersed silicic acids, high molecular weight fatty acids (such as stearic acid), gelatine, agar agar, calcium phosphate, magnesium stearate, animal and vegetable fats, and solid high molecular weight polymers, such as polyethylene glycol.

Oily suspensions for parenteral or topical applications can be vegetable synthetic or semisynthetic oils, such as liquid fatty acid esters having in each case from 8 to 22 C atoms in the fatty acid chains, for example palmitic acid, lauric acid, tridecanoic acid, margaric acid, stearic acid, arachidic acid, myristic acid, behenic acid, pentadecanoic acid, linoleic acid, elaidic acid, brasidic acid, erucic acid or oleic acid, which are esterified with monohydric to trihydric alcohols having from 1 to 6 C atoms, such as methanol, ethanol, propanol, butanol, pentanol or their isomers, glycol or glycerol. Examples of such fatty acid esters are commercially available miglyols, isopropyl myristate, isopropyl palmitate, isopropyl stearate, PEG 6-capric acid, caprylic/capric acid esters of saturated fatty alcohols, polyoxyethylene glycerol trioleates, ethyl oleate, waxy fatty acid esters, such as artificial ducktail gland fat, coconut fatty acid isopropyl ester, oleyl oleate, decyl oleate, ethyl lactate, dibutyl phthalate, diisopropyl adipate, polyol fatty acid esters, inter alia. Silicone oils of differing viscosity, or fatty alcohols, such as isotridecyl alcohol, 2-octyldodecanol, cetylstearyl alcohol or oleyl alcohol, or fatty acids, such as oleic acid, are also suitable. It is furthermore possible to use vegetable oils, such as castor oil, almond oil, olive oil, sesame oil, cotton seed oil, groundnut oil or soybean oil.

Suitable solvents, gelatinizing agents and solubilizers are water or watermiscible solvents. Examples of suitable substances are alcohols, such as ethanol or isopropyl alcohol, benzyl alcohol, 2-octyldodecanol, polyethylene glycols, phthalates, adipates, propylene glycol, glycerol, di- or tripropylene glycol, waxes, methyl cellosolve, cellosolve, esters, morpholines, dioxane, dimethyl sulphoxide, dimethylformamide, tetrahydrofuran, cyclohexanone, etc.

Cellulose ethers which can dissolve or swell both in water or in organic solvents, such as hydroxypropylmethyl cellulose, methyl cellulose or ethyl cellulose, or soluble starches, can be used as film-forming agents.

Mixtures of gelatinizing agents and film-forming agents are also perfectly possible. In this case, use is made, in particular, of ionic macromolecules such as sodium carboxymethyl cellulose, polyacrylic acid, polymethacrylic acid and their salts, sodium amylopectin semiglycolate, alginic acid or propylene glycol alginate as the sodium salt, gum arabic, xanthan gum, guar gum or carrageenan. The following can be used as additional formulation aids: glycerol, paraffin of differing viscosity, triethanolamine, collagen, allantoin and novantisolic acid. Use of surfactants, emulsifiers or wetting agents, for example of Na lauryl sulphate, fatty alcohol ether sulphates, di-Na-N-lauryl-β-iminodipropionate, polyethoxylated castor oil or sorbitan monooleate, sorbitan monostearate, polysorbates (e.g. Tween), cetyl alcohol, lecithin, glycerol monostearate, polyoxyethylene stearate, alkylphenol polyglycol ethers, cetyltrimethylammonium chloride or mono-/dialkylpolyglycol ether orthophosphoric acid monoethanolamine salts can also be required for the formulation. Stabilizers, such as montmorillonites or colloidal silicic acids, for stabilizing emulsions or preventing the breakdown of active substances such as antioxidants, for example tocopherols or butylhydroxyanisole, or preservatives, such as p-hydroxybenzoic acid esters, can likewise be used for preparing the desired formulations.

Preparations for parenteral administration can be present in separate dose unit forms, such as ampoules or vials. Use is preferably made of solutions of the active compound, preferably aqueous solution and, in particular, isotonic solutions and also suspensions. These injection forms can be made available as ready-to-use preparations or only be prepared directly before use, by mixing the active compound, for example the lyophilisate, where appropriate containing other solid carrier substances, with the desired solvent or suspending agent.

Intranasal preparations can be present as aqueous or oily solutions or as aqueous or oily suspensions. They can also be present as lyophilisates which are prepared before use using the suitable solvent or suspending agent.

Inhalable preparations can present as powders, solutions or suspensions. Preferably, inhalable preparations are in the form of powders, e.g. as a mixture of the active ingredient with a suitable formulation aid such as lactose.

The preparations are produced, aliquoted and sealed under the customary antimicrobial and aseptic conditions. As indicated above, the compounds of the invention may be administered as a combination therapy with further active agents, e.g. therapeutically active compounds useful in the treatment of central nervous system disorders.

These further compounds may be PDE10 inhibitors or compounds which have an activity which is not based on PDE10 inhibition such as dopamine D2 receptor modulating agents or NMDA modulating agents.

For a combination therapy, the active ingredients may be formulated as compositions containing several active ingredients in a single dose form and/or as kits containing individual active ingredients in separate dose forms. The active ingredients used in combination therapy may be coadministered or administered separately.

### Pharmaceutical Methods

Compounds of the invention or pharmaceutically acceptable salts of the compounds are phosphodiesterase 10 inhibitors which are useful in treating or preventing disorders caused by, associated with and/or accompanied by phosphodiesterase 10 hyperactivity and/or disorders such as central nervous system disorders.

In one aspect, the present invention relates to the treatment of neurological disorders and psychiatric disorders including, but not limited to, schizophrenia and other psychotic disorders; mood [affective] disorders; neurotic, stress-related and somatoform including anxiety disorders; eating disorders; sexual dysfunction; excessive sexual drive; disorders of adult personality and behavior; disorders usually first diagnosed in infancy, childhood or adolescence; mental retardation; disorders of psychological development; disorders comprising the symptom of cognitive deficiency in a mammal, including a human; and factitious disorders.

Exemplary schizophrenia and other psychotic disorders that can be treated according to the present invention include, but are not limited to, continuous or episodic schizophrenia of different types (for instance, paranoid, hebephrenic, catatonic, undifferentiated, residual, and schizophreniform disorders); schizotypal disorders (such as borderline, latent, prepsychotic, prodromal, pseudoneurotic pseudopsychopathic schizophrenia and schizotypal personality disorder); persistent delusional disorders; induced acute, transient and persistent psychotic disorders; induced delusional disorders; schizoaffective disorders of different types (for instance, manic depressive or mixed type); puerperal psychosis, and other nonorganic psychosis.

Exemplary mood [affective] disorders that can be treated according to the present invention include, but not limited to, manic episodes associated with bipolar disorder and single manic episodes; hypomania; mania with psychotic symptoms; bipolar affective disorders (including for instance bipolar affective disorders with current hypomanic and manic episodes with or without psychotic symptoms, bipolar I disorder or bipolar II disorder); depressive disorders, such as single episode or recurrent major depressive disorder of the mild moderate or severe type; depressive disorder with postpartum onset; depressive disorders with psychotic symptoms; persistent mood [affective] disorders; cyclothymia; dysthymia; and premenstrual dysphoric disorder.

Exemplary neurotic, stress-related and somatoform disorders that can be treated according to the present invention include, but not limited to, phobic anxiety disorders; agoraphobia and social phobia related to psychosis; anxiety disorders; panic disorders; general anxiety disorders; obsessive compulsive disorder; reaction to severe stress and adjustment disorders; post traumatic stress disorder; dissociative disorders; neurotic disorders; and depersonalisation-derealisation syndrome.

Exemplary the disorders of adult personality and behavior that can be treated according to the present invention include, but not limited to, specific personality disorders of the paranoid, schizoid, schizotypal, antisocial, borderline, histrionic, narcissistic, avoidant, dissocial, emotionally unstable, anankastic, anxious and dependent type; mixed personality disorders; habit and impulse disorders (such as trichotillomania, pyromania, maladaptive aggression); and disorders of sexual preference.

Exemplary disorders usually first diagnosed in infancy, childhood or adolescence that can be treated according to the present invention include, but not limited to, hyperkinetic disorders; attentional deficit/hyperactivity disorder (AD/HD); conduct disorders; mixed disorders of conduct and emotional disorders; nonorganic enuresis; nonorganic encopresis; stereotyped movement disorder; and specified behavioural emotional disorders; attention deficit disorder without hyperactivity; excessive masturbation; nail-biting; nose-picking and thumb-sucking; disorders of psychological development; schizoid disorder of childhood; pervasive development disorders; and psychotic episodes associated with Asperger's syndrome.

Exemplary neurological disorders include neurodegenerative disorders including, without being limited to, Parkinson's disease, Huntington's disease, dementia (for example Alzheimer's disease, multi-infarct dementia, AIDS-related dementia, or fronto temperal dementia), neurodegeneration associated with cerebral trauma, neurodegeneration associated with stroke, neurodegeneration associated with cerebral infarct, hypoglycemia-induced neurodegeneration, neurodegeneration associated with epileptic seizure, neurodegeneration associated with neurotoxic poisoning or multi-system atrophy.

Exemplary disorders of psychological development that can be treated according to the present invention include, but not limited to, developmental disorders of speech and language; developmental disorders of scholastic skills; specific disorder of arithmetical skills; reading disorders and spelling disorders and other learning disorders, which disorders are predominantly diagnosed in infancy, childhood or adolescence.

The phrase “cognitive deficiency” as used here refers to a subnormal functioning or a suboptimal functioning in one or more cognitive aspects such as memory, intellect, learning and logic ability, or attention in a particular individual comparative to other individuals within the same general age population.

Exemplary disorders comprising as a symptom cognitive deficiency that can be treated according to the present invention include, but not limited to, cognitive deficits related to psychosis including schizophrenia; depression; age-associated memory impairment; autism; autistic spectrum disorders; fragile X syndrome; Parkinson's disease; Alzheimer's disease; multi infarct dementia; spinal cord injury; CNS hypoxia; Lewis body dementia; stroke; frontotemporal dementia; progressive supranuclear palsy Huntington's disease and in HIV disease; cerebral trauma; cardiovascular disease; drug abuse; diabetes associated cognitive impairment; and mild cognitive disorder.

In other aspects, the present invention relates to the treatment of movement disorders with malfunction of basal ganglia. Exemplary movement disorders with malfunction of basal ganglia that can be treated according to the present invention include, but not limited to, different subtypes of dystonia, such as focal dystonias, multiple-focal or segmental dystonias, torsion dystonia (induced by psychopharmacological drugs), hemispheric, generalised and tardive dyskinesias, akathisias, dyskinesias such as Huntington's disease, Parkinson's disease, Lewis body disease, restless leg syndrome, PLMS.

In other aspects, the present invention relates to the treatment of organic disorders. Examples of organic disorders include, but not limited to, symptomatic mental disorders, organic delusional (schizophrenia-like) disorders; presenil or senile psychosis associated with dementia; psychosis in epilepsy and Parkinson's disease and other organic and symptomatic psychosis; delirium; infective psychosis; and personality and behavioural disorders due to brain disease, damage and dysfunction.

In another aspect, the present invention relates to the treatment of mental and behavioural disorders due to psychoactive compounds, more particular to the treatment of psychotic disorders and residual and late-onset psychotic disorders induced by alcohol, opioids, cannabinoids, cocaine, hallucinogens, other stimulants, including caffeine, volatile solvents and other psychoactive compounds.

In a further aspect, the present invention relates to a general improvement of learning and memory capacities in a mammal, including a human.

Compounds currently used to treat schizophrenia have been associated with several undesirable side effects. These side effects include weight gain, hyperprolactinemia, elevated triglyceride levels, metabolic syndrome (markers: diabetes, hyperlipidemia, hypertension, and obesity), glucose abnormalities (such as hyperglycemia, elevated blood glucose and impaired glucose tolerance), and the exhibition of extrapyramidal symptoms. The weight gain observed with conventional atypical antipsychotics, such as risperidone and olanzapine, has been associated with an increased risk of cardiovascular disease and diabetes mellitus.

In contrast, compounds of the present invention are useful in treating schizophrenia to effect a clinically relevant improvement such as reduction of a PANSS total score in a patient, while maintaining body weight, maintaining or improving glucose levels and/or tolerance, maintaining and/or improving triglycerides levels and/or total cholesterol levels and/or maintaining an EPS profile similar to baseline measurements before administration.

The PDE10 inhibitors of the invention are further useful in the prevention and treatment of obesity, type 2 diabetes (non-insulin dependent diabetes), metabolic syndrome, glucose intolerance, and related health risks, symptoms or disorders. As such, the compounds can also be used to reduce body fat or body weight of an overweight or obese individual. In some embodiments, the PDE10 inhibitor is selective for PDE 10, meaning that it is a better inhibitor of PDE 10 than for any other PDE. In some embodiments, the selective PDE10 inhibitor can reduce PDE10 activity at least 10-fold or at least 100-fold compared to other PDE's.

As used herein, the terms “overweight” and “obese” are meant to refer to adult persons 18 years or older having a greater than ideal body weight (or body fat) measured by the body mass index (BMI). BMI is calculated by weight in kilograms divided by height in meters squared (kg/m²) or, alternatively, by weight in pounds, multiplied by 703, divided by height in inches squared (lbs×703/in²). Overweight individuals typically have a BMI of between 25 and 29, whereas obsess individuals typically have a BMI of 30 or more (see, e.g., National Heart, Lung, and Blood institute, Clinical Guidelines on the Identification, Evaluation, and Treatment of Overweight and Obesity in Adults, The Evidence Report, Washington, D.C.: U.S. Department of Health and Human Services, NIH publication no. 98-4083, 1998). Other means for indicating excess body weight, excess body fat, and obesity include direct measure of body fat and/or waist-to-hip ratio measurements.

The term “metabolic syndrome” is used according to its usual meaning in the art. The American Heart Association characterizes metabolic syndrome as having at least 3 of the 5 below symptoms: 1) Elevated waist circumference (>102 cm (40 inches) in men;

>88 cm (35 inches) in women), 2) Elevated triglycerides (>150 mg/dL (>1.7 mmol/L) or drug treatment for elevated triglycerides), 3) Reduced HDL-C (<40 mg/dL (1.03 mmol/L) in men <50 mg/dL (1.3 mmol/L) in women or drug treatment for reduced HDL-C, 4) Elevated blood pressure (>130/85 mmHg or drug treatment for hypertension), and 5) Elevated fasting glucose (>100 mg/dL or drug treatment for elevated glucose). See, Grundy, S. M. et al., Circulation, 2005, 112(17, e285 (online at circ.ahajournals.org/cgi/reprint/112/17/e285). Metabolic syndrome according to the World Health Organization (See, Alberti et al., Diabet. Med. 15, 539-553, 1998) includes individuals suffering from diabetes, glucose intolerance, low fasting glucose, or insulin resistance plus two or more of 1) High blood pressure (>160/90 mmHg), 2) Hyperlipdemia (triglycerides ≧150 mg/dL or HDL cholesterol <35 mg/dL in men and <39 mg/dL in women), 3) Central obesity (waist-to-hip ratio of >0.90 for men and >0.85 for women or BMI >30 kg/m2), and 4) Microalbuminuria (urinary albumin excretion rate ≧20 μg/min or an albumin-to-creatine ratio ≧20 μg/kg).

The present methods relating to reduction of body fat or body weight, as well as the treatment or prevention of obesity, type 2 diabetes (non-insulin dependent diabetes), metabolic syndrome, glucose intolerance, and related health risks, symptoms or disorders can be carried out by the administration of one or more compounds of the present invention. In some embodiments, one or more additional therapeutic agents can be administered such as anti-obesity agents. Example anti-obesity agents include apolipoprotein-B secretion/microsomal triglyceride transfer protein (apo-B/MTP) inhibitors, 11-beta-hydroxysteroid dehydrogenase-1(11beta-HSD type 1) inhibitors, peptide YY3-36 or analogs thereof, MCR-4 agonists, cholecystokinin-A (CCK-A) agonists, monoamine reuptake inhibitors (such as sibutramine), cannabinoid receptor-1 antagonists (such as rimona an, sympathomimetic agents, P3 adrenergic receptor agonists, 5 dopamine agonists; (such as bromocriptine), melanocyte-stimulating hormone receptor analogs, 5HT_{2C} agonists, melanin concentrating hormone antagonists, leptin (the OB protein), leptin analogs, leptin receptor agonists, galanin antagonists, lipase inhibitors (such as tetrahydrolipstatin, i.e. orlistat), anorectic agents (such as a bombesin agonist), neuropeptide-Y receptor antagonists (e.g., NPY Y5 receptor antagonists, such as the compounds described in U.S. Pat. Nos. 6,566,367; 61,649,624; 61,638,942; 61,605,720; 61,495,569; 61,462,053; 61,388,077; 6,335,345; and 6,326,375; US Pat. Appl. Publ. Nos. 2002/0151456 and 20031036652; and PCT Publication Nos. WO 031010175, WO 03/082190 and receptor agonists or antagonists, orexin receptor antagonists, glucagon-like peptide-1 receptor agonists, ciliary neurotrophic factors, human agouti-related proteins (AGRP), ghrelin receptor antagonists, histamine 3 receptor antagonists or inverse agonists, neuromedin U receptor agonists and the like. Other anti-obesity agents are readily apparent to one of ordinary skill in the art.

Representative methods for using PDE10 inhibitors for the reduction of body fat or body weight, as well as the treatment or prevention of obesity, type 2 diabetes (non-insulin dependent diabetes), metabolic syndrome, glucose intolerance, and related health risks, symptoms are reported in WO 2005/120514.

The present invention also includes method of treating pain conditions and disorders. Examples of such pain conditions and disorders include, but are not limited to, inflammatory pain, hyperalgesia, inflammatory hyperalgesia, migraine, cancer pain, osteoarthritis pain, post-surgical pain, non-inflammatory pain, neuropathic pain, sub-categories of neuropathic pain including peripheral neuropathic pain syndromes, chemotherapy-induced neuropathy, complex regional pain syndrome, HIV sensory neuropathy, neuropathy secondary to tumor infiltration, painful diabetic neuropathy, phantom limb pain, postherpetic neuralgia, postmastectomy pain, trigeminal neuralgia, central neuropathic pain syndromes, central poststroke pain, multiple sclerosis pain, Parkinson disease pain, and spinal cord injury pain.

In a further embodiment compounds of the present invention are administered in combination with one or more other agents effective for treating pain. Such agents include analgesics, non-steroidal anti-inflammatory drugs (NSAIDs), opiods and antidepressants. In various embodiments, one or more agents are selected from the group consisting of buprenorphine, naloxone, methadone, levomethadyl acetate, L-alpha acetylmethadol (LAAM), hydroxyzine, diphenoxylate, atropine, chlordiazepoxide, carbamazepine, mianserin, benzodiazepine, phenoziazine, disulfuram, acamprosate, topiramate, ondansetron, sertraline, bupropion, amantadine, amiloride, isradipine, tiagabine, baclofen, propranolol, tricyclic antidepressants, desipramine, carbamazepine, valproate, lamotrigine, doxepin, fluoxetine, imipramine, moclobemide, nortriptyline, paroxetine, sertraline, tryptophan, venlafaxine, trazodone, quetiapene, zolpidem, zopiclone, zaleplon, gabapentin, memantine, pregabalin, cannabinoids, tramadol, duloxetine, milnacipran, naltrexone, paracetamol, metoclopramide, loperamide, clonidine, lofexidine, and diazepam.

The present invention also includes methods of treating schizophrenia and other psychotic disorders, as described above, with a combination of compounds of the present invention with one or more antipsychotic agents. Examples of suitable antipsychotic agents for use in combination with the compounds of the present invention include, but are not limited to, the phenothiazine (chlorpromazine, mesoridazine, thioridazine, acetophenazine, fluphenazine, perphenazine and trifluoperazine), thioxanthine (chlorprothixene, thiothixene), heterocyclic dibenzazepine (clozapine, olanzepine and aripiprazole), butyrophenone (haloperidol), dipheyylbutylpiperidine (pimozide) and indolone (molindolone) classes of antipsychotic agents. Other antipsychotic agents with potential therapeutic value in combination with the compounds in the present invention include loxapine, sulpiride and risperidone.

The present invention further includes methods of treating depression or treatment-resistant depression with a combination of compounds of the present invention with one or more antidepressants. Examples of suitable anti-depressants for use in combination with the compounds of the present invention include, but are not limited to, norepinephrine reuptake inhibitors (tertiary and secondary amine tricyclics), selective serotonin reuptake inhibitors (SSRIs) (e.g., fluoxetine, fluvoxamine, paroxetine and sertraline), monoamine oxidase inhibitors (MAOIs) (isocarboxazid, phenelzine, tranylcypromine, selegiline), reversible inhibitors of monoamine oxidase (RIMAs) (moclobemide), serotonin and norepinephrine reuptake inhibitors (SNRIs) (venlafaxine), corticotropin releasing factor (CRF) receptor antagonists, alpha-adrenoreceptor antagonists, and atypical antidepressants (bupropion, lithium, nefazodone, trazodone and viloxazine).

In order that the invention disclosed herein may be more efficiently understood, examples are provided below. It should be understood that these examples are for illustrative purposes only and are not to be construed as limiting the invention in any manner.

### EXAMPLES

Scheme 3 shows a synthetic method that was used in the preparation of compounds of examples 1-4.

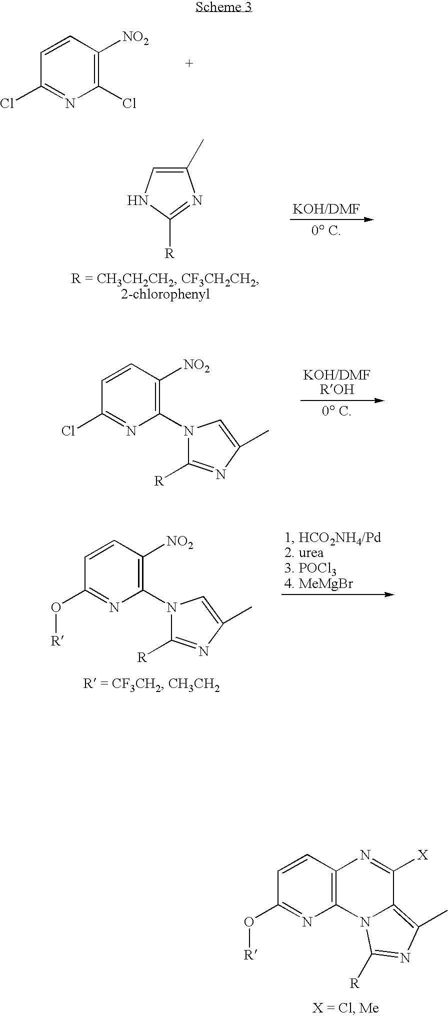

### Example 1

### 6-Chloro-7-methyl-2-(2,2,2-trifluoroethoxy)-9-(3,3,3-trifluoropropyl)imidazo[1,5-a]pyrido[3,2-e]pyrazine

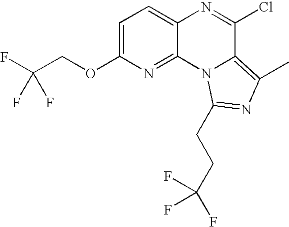

### Step 1

### 4-Methyl-2-(3,3,3-trifluoro-propyl)-1H-imidazole

Concentrated NH₄OH (2.1 mL) and water (4.2 mL) were combined and stirred. To this was added 4,4,4-trifluoro-butyraldehyde (3.5 g, 28 mmol) dissolved in methanol (7 mL). The reaction was let stir 10 min at room temperature and a 40% solution of methylglyoxal (6 mL, 31 mmol) dissolved water (6 mL) was added in one portion. The reaction was heated to 35° C. for 1 hr then stirred at room temperature overnight and extracted with CHCl₃3×. The extracts were separated and combined then brined and dried over Na₂SO₄. After filtration, the solvent was removed under reduced pressure. The crude was purified by flash chromatography on silica gel in ethyl acetate. A pale yellow oil was recovered (2.1 g) 42% yield. MS (ES) m/z 179.1 [M+1]⁺

### Step 2

### 6-Chloro-2-[4-methyl-2-(3,3,3-trifluoro-propyl)-imidazol-1-yl]-3-nitro-pyridine

4-Methyl-2-(3,3,3-trifluoro-propyl)-1H-imidazole (Example 1, step 1) (1.5 g, 8.4 mmol) was dissolved in DMF (25 mL) and cooled to 0° C. To this was added powdered KOH (0.49 g, 9.2 mmol). The reaction was stirred for 5 min and 2,6-dichloro-3-nitropyridine (1.6 g, 8.4 mmol) was added in one portion. The reaction was let stir at 0° C. for 3 hrs then diluted with water and extracted with ether. The extracts were separated and combined, washed with water, then brined and dried over Na₂SO₄. After filtration, the solvent was removed under reduced pressure. The crude was purified by flash chromatography on silica gel in hexane/ethyl acetate 1:1. A brown solid was recovered (0.8 g) 28% yield. MS (ES) m/z 335.1 [M+1]⁺

### Step 3

### 2-[4-Methyl-2-(3,3,3-trifluoro-propyl)-imidazol-1-yl]-3-nitro-6-(2,2,2-trifluoro-ethoxy)-pyridine

6-Chloro-2-[4-methyl-2-(3,3,3-trifluoro-propyl)-imidazol-1-yl]-3-nitro-pyridine (Example 1, Step 2) (1.4 g, 4.2 mmol) was dissolved in DMF (14 mL) and cooled to 0° C. To this was added powdered KOH (0.23 g, 4.2 mmol). The reaction was stirred for 5 min and 2,2,2-trifluoroethanol (0.3 mL, 4.2 mmol) was added in one portion. The reaction was let stir at 0° C. for 3 hrs then diluted with water and extracted with ethyl acetate. The extracts were separated and combined, washed with water, then brined and dried over MgSO₄. After filtration, the solvent was removed under reduced pressure. The crude was purified by flash chromatography on silica gel in hexane/ethyl acetate 2:1. A brown solid was recovered (0.38 g) 23% yield. MS (ES) m/z 399.1 [M+1]⁺

### Step 4

### 2-[4-Methyl-2-(3,3,3-trifluoro-propyl)-imidazol-1-yl]-6-(2,2,2-trifluoro-ethoxy)-pyridin-3-ylamine

2-[4-Methyl-2-(3,3,3-trifluoro-propyl)-imidazol-1-yl]-3-nitro-6-(2,2,2-trifluoro-ethoxy)-pyridine (Example 1, Step 3) (0.34 g, 0.85 mmol) and 10% Pd/C (0.048 g, 5% mol) were combined in 20 mL flask (connected with a condenser) and loaded 4 mL THF, followed by slow addition of 4 mL MeOH with stirring. Ammonium formate (0.296 g, 4.6 mmol) was added in one portion into the stirring mixture and the final mixture was stirred at room temperature for 10 min (gas released) then warmed to 50° C. for 1 hr. The reaction was cooled to room temperature and filtered through celite. The solvent was evaporated by rotovap and the residue partitioned between water and ethyl acetate. The aqueous phase was extracted with ethyl acetate and dried over MgSO₄. After filtration, the solvent was removed under reduced pressure. The crude was purified by flash chromatography on silica gel in hexane/ethyl acetate 1:1. A white solid was recovered (0.21 g) 68% yield. MS (ES) m/z 369.1 [M+1]⁺

### Step 5

### 3-Methyl-8-(2,2,2-trifluoro-ethoxy)-1-(3,3,3-trifluoro-propyl)-5H-2,5,9,9b-tetraaza-cyclopenta[a]naphthalen-4-one

A mixture of 2-[4-Methyl-2-(3,3,3-trifluoro-propyl)-imidazol-1-yl]-6-(2,2,2-trifluoro-ethoxy)-pyridin-3-ylamine (Example 1, Step 4) (0.2 g, 0.54 mmol) and urea (0.46 g, 7.5 mmol) were heated to 160° C. The reaction mixture was stirred for 2 hrs and glacial acetic acid (0.12 mL, 1.9 mmol) added. The stirring was continued for further 6 hrs. The reaction mixture was allowed to cool to 70° C. then diluted with water and stirred for 1 hr at 50° C. The warm mixture was filtered and the solids washed with water then dried. A tan solid was recovered (0.15 g) 70% yield.

### Step 6

### 6-chloro-7-methyl-2-(2,2,2-trifluoroethoxy)-9-(3,3,3-trifluoropropyl)imidazo[1,5-a]pyrido[3,2-e]pyrazine

3-Methyl-8-(2,2,2-trifluoro-ethoxy)-1-(3,3,3-trifluoro-propyl)-5H-2,5,9,9b-tetraaza-cyclopenta[a]naphthalen-4-one (0.1 g, 0.25 mmol) (Example 1, Step 5) was dissolved in phosphorous oxychloride (1.5 mL) and heated to 120° C. for 4 hrs. The reaction was poured onto ice and neutralized with sodium bicarbonate. The aqueous solution was then extracted with ethyl acetate. The organic layers were separated and combined then washed with water, brined and dried over MgSO₄. After filtration, the solvent was removed under reduced pressure. The crude was purified by flash chromatography on silica gel in hexane/ethyl acetate 10:2. A yellow solid was recovered (0.029 g) 28% yield. MS (ES) m/z 413.1 [M+1]⁺

### Example 2

### 6-Chloro-2-ethoxy-7-methyl-9-propylimidazo[1,5-a]pyrido[3,2-e]pyrazine

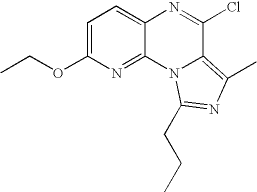

6-Chloro-2-ethoxy-7-methyl-9-propylimidazo[1,5-a]pyrido[3,2-e]pyrazine was prepared in a manner similar to Example 1 starting with butyraldehyde (2 g, 28 mmol). A yellow solid was recovered (0.016 g) 19% yield overall. MS (ES) m/z 305.1 [M+1]⁺

### Example 3

### 2-Ethoxy-6,7-dimethyl-9-propylimidazo[1,5-a]pyrido[3,2-e]pyrazine

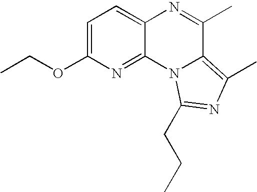

6-Chloro-2-ethoxy-7-methyl-9-propylimidazo[1,5-a]pyrido[3,2-e]pyrazine (0.1 g, 0.33 mmol) was dissolved in dry THF (3 mL). To this was added methyl magnesium bromide (3M/ether) (0.44 mL, 1.3 mmol). The reaction was let to stir at room temperature over night then poured into saturated ammonium chloride and extracted with ethyl acetate. The organic layers were separated and combined then washed with water, brined and dried over MgSO₄. After filtration, the solvent was removed under reduced pressure. The crude was purified by flash chromatography on silica gel in hexane/ethyl acetate 2:1. A yellow solid was recovered (0.06 g) 64% yield. MS (ES) m/z 285.1 [M+1]⁺

### Example 4

### 9-(2-Chlorophenyl)-2-ethoxy-6,7-dimethylimidazo[1,5-a]pyrido[3,2-e]pyrazine

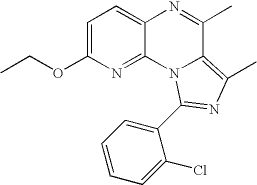

### Step 1

### 2-(2-Chloro-phenyl)-4-methyl-1H-imidazole

To concentrated NH₄OH (4 mL) was added 2-chlorobenzaldehyde (1.0 g, 7.1 mmol) dissolved in ethanol (4 mL). The reaction was heated to 50° C. and a 40% solution of methylglyoxal (1.6 mL, 8.9 mmol) was added in one portion. The reaction temperature was maintained with stirring for 3 hrs then diluted with water and extracted with ethyl acetate. The extracts were separated and combined then brined and dried over MgSO₄. After filtration, the solvent was removed under reduced pressure. The crude triturated with ethanol and filtered. A tan solid was recovered (0.41 g) 30% yield. MS (ES) m/z 193.1 [M+1]⁺

### Step 2

### 9-(2-Chlorophenyl)-2-ethoxy-6,7-dimethylimidazo[1,5-a]pyrido[3,2-e]pyrazine

9-(2-Chlorophenyl)-2-ethoxy-6,7-dimethylimidazo[1,5-a]pyrido[3,2-e]pyrazine was prepared in a manner similar to Example 3 starting with 2-(2-chloro-phenyl)-4-methyl-1H-imidazole (Example 4, step 1) (0.41 g, 2.1 mmol). A yellow solid was recovered (0.05 g) 2% yield overall. MS (ES) m/z 353.1 [M+1]⁺

Examples 5-11 were prepared according to the following synthetic scheme (Scheme 4).

### Method A

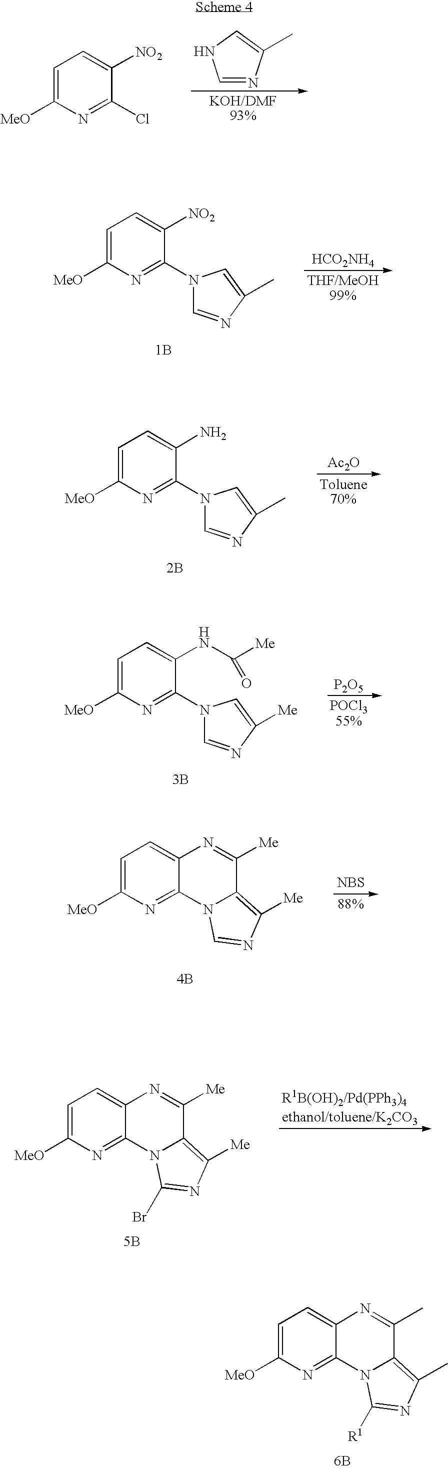

### 6-Methoxy-2-(4-methyl-1H-imidazol-1-yl)-3-nitropyridine (1B)

To a N,N-dimethylformamide (500 mL) solution of 4-methylimidazole (8.5 g, 103 mmol) was added freshly powdered KOH (6.72 g, 120 mmol) in two portions under N₂ at 0° C., followed by addition of 2-chloro-6-methoxy-3-nitropyridine (18.9 g, 100 mmol). The resulting solution was warmed to room temperature and stirred for 2 hours. Majority of solvent was removed under vacuum and the residue was diluted with water and extracted with ethyl acetate three times. The organic layer was combined and washed two more times with water to remove additional dimethyl formamide and dried over magnesium sulfate. Solvent was evaporated under vacuum and the residue was purified by column (15-25% gradient eluent of ethyl acetate in dichloromethane) to provide compound 1B as a yellow oil (21.9 g, 93% yield) which becomes yellow solid after standing on bench.

¹H NMR (400 MHz, DMSO) δ ppm 8.48 (d, 1H), 8.00 (s, 1H), 7.18 (s, 1H), 7.01 (d, 1H), 3.97 (s, 3H), 2.12 (s, 3H); EIMS 235.0 [M+H]+.

### 6-Methoxy-2-(4-methyl-1H-imidazol-1-yl)pyridin-3-amine (2B)

To a mixture of intermediate (1B) (21.4 g, 91.5 mmol) and 10% Pd/C (5.12 g, 4.58 mmol) in a 1 L RB flask (connected with a condenser) was loaded 240 mL THF, followed by slow addition of 240 mL MeOH under N₂ with stirring. HCOONH₄ (34.75 g, 503.25 mmol) was added in two portions into the stirring mixture and the final mixture was stirred at room temperature for 10 min (gas released) and then warmed to 50° C. for 1 hr. The reaction was cooled to room temperature and filtered through celite. Solvent was evaporated under vacuum to dryness to provide a clean product as an offwhite powder (18.6 g, 99% yield). NMR indicated a 4:1 ratio mixture of two regioisomers with the major one as the desired regioisomer (confirmed by NOE studies).

¹H NMR (400 MHz, DMSO) δ ppm 7.91 (s, 1H), 7.30 (d, 1H), 7.25 (s, 1H), 6.63 (d, 1H), 4.70 (s, br, 2H), 3.70 (s, 3H), 2.13 (s, 3H); EIMS 205.0 [M+H]+.

### N-(6-Methoxy-2-(4-methyl-1H-imidazol-1-yl)pyridin-3-yl)acetamide (3B)

To a solution of intermediate (2B) (8.16 g, 40 mmol, 4:1 mix) in 200 mL toluene was added acetic anhydride (18.8 mL, 200 mmol) in dropwise. The resulting mixture was stirred at room temperature for 3.5 hours. Stop the agitation for 30 min and the precipitate was filtered to provide a product as an offwhite solid 5.45 g (70% yield based on the major isomer) as a single regioisomer.

¹H NMR (400 MHz, DMSO) δ ppm 9.58 (s, 1H), 8.00 (s, 1H), 7.72 (d, 1H), 7.30 (s, 1H), 6.80 (d, 1H), 3.84 (s, 3H), 2.12 (s, 3H), 1.95 (s, 3H); EIMS 247.1 [M+H]+.

### 2-Methoxy-6,7-dimethylimidazo[1,5-a]pyrido[3,2-e]pyrazine (4B)

To a solution of intermediate (3B) (2.04 g, 8.2 mmol) in 16 mL of POCl₃ was added P₂O₅ quickly (minimize the moisture induction). The resulting mixture was refluxed at 110-120° C. for 4 hours. POCl₃ was evaporated and the residue was quenched with ice-water very carefully. The mixture was neutralized with saturated Na₂CO₃ solution and extracted with ethyl acetate. The organic layer was dried over magnesium sulfate. Condensation followed by column chromatography using 2-5% MeOH in dichloromethane as eluent to provide a product as a yellow powder 1.12 g (55% yield).

¹H NMR (400 MHz, DMSO) δ ppm 8.82 (s, 1H), 8.07 (d, 1H), 6.95 (d, 1H), 3.99 (s, 3H), 2.69 (s, 3H), 2.64 (s, 3H); EIMS 229.0 [M+H]+.

### 9-Bromo-2-methoxy-6,7-dimethylimidazo[1,5-a]pyrido[3,2-e]pyrazine (5B)

To a mixture of intermediate (4B) (172 mg, 0.75 mmol) and NBS (200 mg, 1.13 mmol) was added anhydrous CH₃CN (6 mL) under N₂. The resulting solution was stirred in dark for 24 hours. The reaction was concentrated to dryness and the residue was dissolved in 30 mL ethyl acetate. The solution was washed twice with brine (2×30 mL), saturated Na₂SO₃ solution (20 mL) and brine (20 mL). All aqueous phase were combined and extracted with ethyl acetate (2×50 mL). The organic layers were combined and dried over magnesium sulfate. Evaporation under vacuum to dryness to provide a clean product as a light yellow powder (206 mg, 88% yield).

¹H NMR (400 MHz, DMSO) δ ppm 8.08 (d, 1H), 7.01 (d, 1H), 4.04 (s, 3H), 2.67 (s, 3H), 2.62 (s, 3H); EIMS 306.9 [M+H]+.

### Example 5

### 9-(3-Fluorophenyl)-2-methoxy-6,7-dimethylimidazo[1,5-a]pyrido[3,2-e]pyrazine

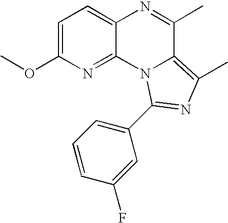

9-Bromo-2-methoxy-6,7-dimethylimidazo[1,5-a]pyrido[3,2-e]pyrazine 5B (0.12 g, 0.39 mmol) was suspended in a solution containing ethanol (2 mL) and toluene (2 mL). To this was added 3-fluorophenylboronic acid (0.12 g, 0.72 mmol) followed by potassium carbonate (0.15 g, 1.4 mmol) and tetrakis(triphenylphosphine)palladium(0) (0.023 g, 5% mole). After bubbling argon thru the reaction for 1 min, the reaction was sealed and heated to 110° C. overnight. The reaction was then removed of solvent under reduced pressure. The crude was purified by flash chromatography on silica gel in hexane/ethyl acetate 1:1. A tan solid was recovered (0.06 g) 47% yield. MS (ES) m/z 323.2 [M+1]⁺

### Example 6

### 9-(3,5-Dichlorophenyl)-2-methoxy-6,7-dimethylimidazo[1,5-a]pyrido[3,2-e]pyrazine

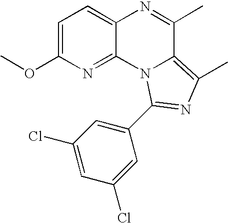

9-(3,5-Dichlorophenyl)-2-methoxy-6,7-dimethylimidazo[1,5-a]pyrido[3,2-e]pyrazine 5B synthesized in a manner similar to compound 5, starting with 9-bromo-2-methoxy-6,7-dimethylimidazo[1,5-a]pyrido[3,2-e]pyrazine (0.12 g, 0.39 mmol) and 3,5-dichlorophenylboronic acid (0.13 g, 0.72 mmol) The crude was purified by flash chromatography on silica gel in hexane/ethyl acetate 1:1. A tan solid was recovered (0.08 g) 55% yield. MS (ES) m/z 373.1 [M+1]⁺

### Example 7

### 9-(3,4-Dichlorophenyl)-2-methoxy-6,7-dimethylimidazo[1,5-a]pyrido[3,2-e]pyrazine

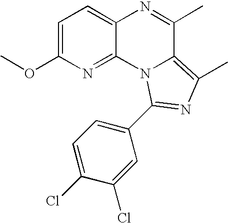

9-(3,4-Dichlorophenyl)-2-methoxy-6,7-dimethylimidazo[1,5-a]pyrido[3,2-e]pyrazine synthesized in a manner similar to Example 5, starting with 9-bromo-2-methoxy-6,7-dimethylimidazo[1,5-a]pyrido[3,2-e]pyrazine 5B (0.12 g, 0.39 mmol) and 3,4-dichlorophenylboronic acid (0.13 g, 0.72 mmol) The crude was purified by flash chromatography on silica gel in hexane/ethyl acetate 1:1. A tan solid was recovered (0.09 g) 62% yield. MS (ES) m/z 373.1 [M+1]⁺

### Example 8

### 9-(2,4-Difluorophenyl)-2-methoxy-6,7-dimethylimidazo[1,5-a]pyrido[3,2-e]pyrazine

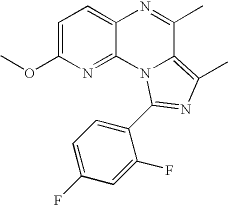

9-(2,4-Difluorophenyl)-2-methoxy-6,7-dimethylimidazo[1,5-a]pyrido[3,2-e]pyrazine synthesized in a manner similar to Example 5, starting with 9-bromo-2-methoxy-6,7-dimethylimidazo[1,5-a]pyrido[3,2-e]pyrazine 5B (0.12 g, 0.39 mmol) and 1,4-difluororophenylboronic acid (0.11 g, 0.72 mmol) The crude was purified by flash chromatography on silica gel in hexane/ethyl acetate 1:1. A yellow solid was recovered (0.04 g) 30% yield. MS (ES) m/z 341.1 [M+1]⁺

### Example 9

### 9-(6-Fluoropyridin-3-yl)-2-methoxy-6,7-dimethylimidazo[1,5-a]pyrido[3,2-e]pyrazine

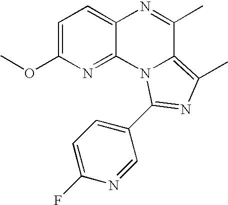

9-(6-Fluoropyridin-3-yl)-2-methoxy-6,7-dimethylimidazo[1,5-a]pyrido[3,2-e]pyrazine synthesized in a manner similar to Example 5, starting with 9-bromo-2-methoxy-6,7-dimethylimidazo[1,5-a]pyrido[3,2-e]pyrazine 5B (0.12 g, 0.39 mmol) and 2-fluoro-5-pyridineboronic acid (0.1 g, 0.72 mmol) The crude was purified by flash chromatography on silica gel in ethyl acetate. A white solid was recovered (0.07 g) 55% yield. MS (ES) m/z 324.1 [M+1]⁺

### Example 10

### 2-Methoxy-6,7-dimethyl-9-pyridin-3-ylimidazo[1,5-a]pyrido[3,2-e]pyrazine

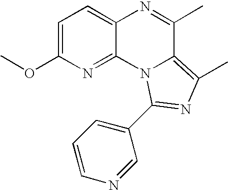

2-Methoxy-6,7-dimethyl-9-pyridin-3-ylimidazo[1,5-a]pyrido[3,2-e]pyrazine synthesized in a manner similar to Example 5, starting with 9-bromo-2-methoxy-6,7-dimethylimidazo[1,5-a]pyrido[3,2-e]pyrazine 5B (0.12 g, 0.39 mmol) and 3-pyridineboronic acid (0.86 g, 0.72 mmol) The crude was purified by flash chromatography on silica gel in ethyl acetate. A white solid was recovered (0.05 g) 42% yield. MS (ES) m/z 306.2 [M+1]⁺

### Example 11

### 2-Methoxy-6,7-dimethyl-9-pyridin-4-ylimidazo[1,5-a]pyrido[3,2-e]pyrazine

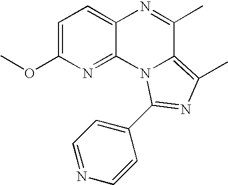

2-Methoxy-6,7-dimethyl-9-pyridin-4-ylimidazo[1,5-a]pyrido[3,2-e]pyrazine synthesized in a manner similar to Example 5, starting with 9-bromo-2-methoxy-6,7-dimethylimidazo[1,5-a]pyrido[3,2-e]pyrazine 5B (0.12 g, 0.39 mmol) and 4-pyridineboronic acid (0.86 g, 0.72 mmol). The crude was purified by flash chromatography on silica gel in ethyl acetate. A white solid was recovered (0.02 g) 17% yield. MS (ES) m/z 306.2 [M+1]⁺

Examples 12-33 were prepared according to the following synthesis (Method B).

### Method B

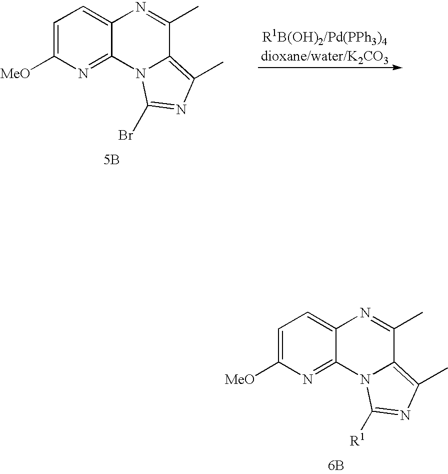

### Example 12

### 9-(2-Chloro-4-methylphenyl)-2-methoxy-6,7-dimethylimidazo[1,5-a]pyrido[3,2-e]pyrazine

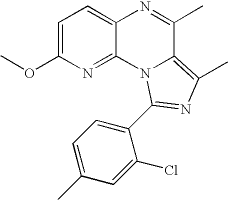

9-Bromo-2-methoxy-6,7-dimethylimidazo[1,5-a]pyrido[3,2-e]pyrazine 5B (0.12 g, 0.39 mmol) was suspended in a solution containing dioxane (4 mL) and water (1 mL). To this was added 2-chlorophenylboronic acid (0.1 g, 1.2 mmol) followed by potassium carbonate (0.15 g, 1.4 mmol) and tetrakis(triphenylphosphine)palladium(0) (0.023 g, 5% mole). After bubbling argon thru the reaction for 1 min, the reaction was sealed and heated to 100° C. overnight. The reaction was then removed of solvent under reduced pressure. The crude was purified by flash chromatography on silica gel in hexane/ethyl acetate 2:1. A white solid was recovered (0.06 g) 44% yield. MS (ES) m/z 353.0 [M+1]⁺

### Example 13

### 9-(4-Chloro-2-methylphenyl)-2-methoxy-6,7-dimethylimidazo[1,5-a]pyrido[3,2-e]pyrazine

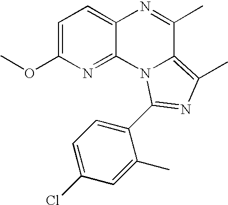

9-(4-Chloro-2-methylphenyl)-2-methoxy-6,7-dimethylimidazo[1,5-a]pyrido[3,2-e]pyrazine was synthesized in a manner similar to Example 12, starting with 9-bromo-2-methoxy-6,7-dimethylimidazo[1,5-a]pyrido[3,2-e]pyrazine 5B (0.12 g, 0.39 mmol) and 2-methyl-4-chlorophenylboronic acid (0.1 g, 0.58 mmol) The crude was purified by flash chromatography on silica gel in ethyl acetate. A white solid was recovered (0.07 g) 51% yield. MS (ES) m/z 353.0 [M+1]⁺

### Example 14

### 9-(2-Fluoro-4-methylphenyl)-2-methoxy-6,7-dimethylimidazo[1,5-a]pyrido[3,2-e]pyrazine

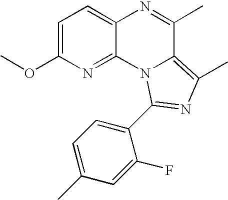

9-(2-Fluoro-4-methylphenyl)-2-methoxy-6,7-dimethylimidazo[1,5-a]pyrido[3,2-e]pyrazine was synthesized in a manner similar to Example 12, starting with 9-bromo-2-methoxy-6,7-dimethylimidazo[1,5-a]pyrido[3,2-e]pyrazine 5B (0.12 g, 0.39 mmol) and 2-fluoro-4-methylphenylboronic acid (0.1 g, 0.58 mmol) The crude was purified by flash chromatography on silica gel in hexane/hyl acetate 2:1. A white solid was recovered (0.09 g) 68% yield. MS (ES) m/z 337.1 [M+1]⁺

### Example 15

### 9-(2-Fluoro-3-methoxyphenyl)-2-methoxy-6,7-dimethylimidazo[1,5-a]pyrido[3,2-e]pyrazine

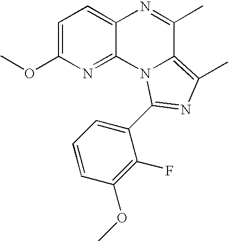

9-(2-Fluoro-3-methoxyphenyl)-2-methoxy-6,7-dimethylimidazo[1,5-a]pyrido[3,2-e]pyrazine was synthesized in a manner similar to Example 12, starting with 9-bromo-2-methoxy-6,7-dimethylimidazo[1,5-a]pyrido[3,2-e]pyrazine 5B (0.12 g, 0.39 mmol) and 2-fluoro-3-methoxyphenylboronic acid (0.1 g, 0.58 mmol) The crude was purified by flash chromatography on silica gel in hexane/ethyl acetate 2:1. A pale yellow solid was recovered (0.02 g) 14% yield. MS (ES) m/z 353.1 [M+1]⁺

### Example 16

### 9-(2-Chloro-4-fluorophenyl)-2-methoxy-6,7-dimethylimidazo[1,5-a]pyrido[3,2-e]pyrazine

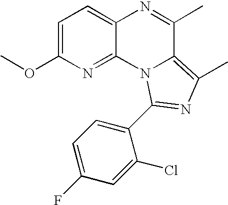

9-(2-Chloro-4-fluorophenyl)-2-methoxy-6,7-dimethylimidazo[1,5-a]pyrido[3,2-e]pyrazine was synthesized in a manner similar to Example 12, starting with 9-bromo-2-methoxy-6,7-dimethylimidazo[1,5-a]pyrido[3,2-e]pyrazine 5B (0.12 g, 0.39 mmol) and 2-chloro-4-fluorophenylboronic acid (0.2 g, 1.2 mmol) The crude was purified by flash chromatography on silica gel in hexane/ethyl acetate 2:1. A pale yellow solid was recovered (0.03 g) 22% yield. MS (ES) m/z 357.0 [M+1]⁺

### Example 17

### 9-(4-Chloro-2-fluorophenyl)-2-methoxy-6,7-dimethylimidazo[1,5-a]pyrido[3,2-e]pyrazine

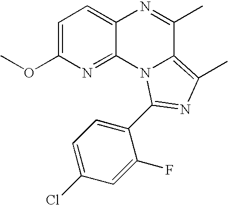

9-(4-Chloro-2-fluorophenyl)-2-methoxy-6,7-dimethylimidazo[1,5-a]pyrido[3,2-e]pyrazine was synthesized in a manner similar to Example 12, starting with 9-bromo-2-methoxy-6,7-dimethylimidazo[1,5-a]pyrido[3,2-e]pyrazine 5B (0.12 g, 0.39 mmol) and 2-fluoro-4-chlorophenylboronic acid (0.2 g, 1.2 mmol) The crude was purified by flash chromatography on silica gel in hexane/ethyl acetate 2:1. A pale yellow solid was recovered (0.08 g) 56% yield. MS (ES) m/z 353.1 [M+1]⁺

### Example 18

### 9-(2-Chloro-4-methoxyphenyl)-2-methoxy-6,7-dimethylimidazo[1,5-a]pyrido[3,2-e]pyrazine

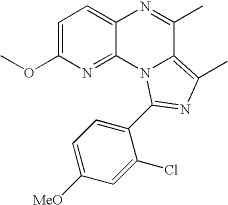

9-(2-Chloro-4-methoxyphenyl)-2-methoxy-6,7-dimethylimidazo[1,5-a]pyrido[3,2-e]pyrazine was synthesized in a manner similar to Example 12, starting with 9-bromo-2-methoxy-6,7-dimethylimidazo[1,5-a]pyrido[3,2-e]pyrazine 5B (0.12 g, 0.39 mmol) and 2-chloro-4-methoxyphenylboronic acid (0.22 g, 1.2 mmol) The crude was purified by flash chromatography on silica gel in hexane/ethyl acetate 2:1. A white solid was recovered (0.06 g) 41% yield. MS (ES) m/z 369.0 [M+1]⁺

### Example 19

### 9-(2-Chloro-5-methoxyphenyl)-2-methoxy-6,7-dimethylimidazo[1,5-a]pyrido[3,2-e]pyrazine

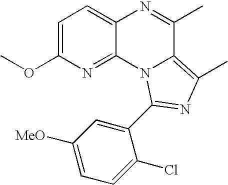

9-(2-Chloro-5-methoxyphenyl)-2-methoxy-6,7-dimethylimidazo[1,5-a]pyrido[3,2-e]pyrazine was synthesized in a manner similar to Example 12, starting with 9-bromo-2-methoxy-6,7-dimethylimidazo[1,5-a]pyrido[3,2-e]pyrazine 5B (0.12 g, 0.39 mmol) and 2-chloro-5-methoxyphenylboronic acid (0.22 g, 1.2 mmol) The crude was purified by flash chromatography on silica gel in hexane/ethyl acetate 2:1. A white solid was recovered (0.07 g) 50% yield. MS (ES) m/z 369.0 [M+1]⁺

### Example 20

### 9-[2-Chloro-4-(trifluoromethyl)phenyl]-2-methoxy-6,7-dimethylimidazo[1,5-a]pyrido[3,2-e]pyrazine

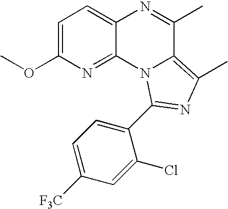

9-[2-Chloro-4-(trifluoromethyl)phenyl]-2-methoxy-6,7-dimethylimidazo[1,5-a]pyrido[3,2-e]pyrazine was synthesized in a manner similar to Example 12, starting with 9-bromo-2-methoxy-6,7-dimethylimidazo[1,5-a]pyrido[3,2-e]pyrazine 5B (0.12 g, 0.39 mmol) and 2-chloro-4-trifluoromethylphenylboronic acid (0.27 g, 1.2 mmol) The crude was purified by flash chromatography on silica gel in hexane/ethyl acetate 2:1. A white solid was recovered (0.1 g) 63% yield. MS (ES) m/z 407.0 [M+1]⁺

### Example 21

### 9-(2-Fluoro-5-methylphenyl)-2-methoxy-6,7-dimethylimidazo[1,5-a]pyrido[3,2-e]pyrazine

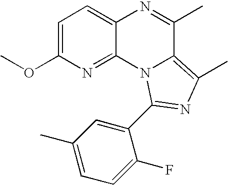

9-(2-Fluoro-5-methylphenyl)-2-methoxy-6,7-dimethylimidazo[1,5-a]pyrido[3,2-e]pyrazine was synthesized in a manner similar to Example 12, starting with 9-bromo-2-methoxy-6,7-dimethylimidazo[1,5-a]pyrido[3,2-e]pyrazine 5B (0.12 g, 0.39 mmol) and 2-fluoro-5-methylphenylboronic acid (0.18 g, 1.2 mmol) The crude was purified by flash chromatography on silica gel in hexane/ethyl acetate 2:1. A white solid was recovered (0.11 g) 83% yield. MS (ES) m/z 337.1 [M+1]⁺

### Example 22

### 9-(2-Chloro-5-fluorophenyl)-2-methoxy-6,7-dimethylimidazo[1,5-a]pyrido[3,2-e]pyrazine

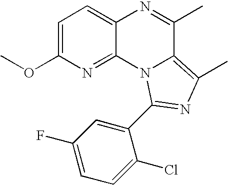

9-(2-Chloro-5-fluorophenyl)-2-methoxy-6,7-dimethylimidazo[1,5-a]pyrido[3,2-e]pyrazine was synthesized in a manner similar to Example 12, starting with 9-bromo-2-methoxy-6,7-dimethylimidazo[1,5-a]pyrido[3,2-e]pyrazine 5B (0.12 g, 0.39 mmol) and 2-chloro-5-fluorophenylboronic acid (0.2 g, 1.2 mmol) The crude was purified by flash chromatography on silica gel in hexane/ethyl acetate 2:1. A white solid was recovered (0.06 g) 43% yield. MS (ES) m/z 357.0 [M+1]⁺

### Example 23

### 9-[2-Chloro-5-(trifluoromethyl)phenyl]-2-methoxy-6,7-dimethylimidazo[1,5-a]pyrido[3,2-e]pyrazine

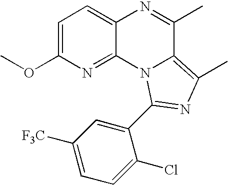

9-[2-Chloro-5-(trifluoromethyl)phenyl]-2-methoxy-6,7-dimethylimidazo[1,5-a]pyrido[3,2-e]pyrazine was synthesized in a manner similar to Example 12, starting with 9-bromo-2-methoxy-6,7-dimethylimidazo[1,5-a]pyrido[3,2-e]pyrazine 5B (0.12 g, 0.39 mmol) and 2-chloro-5-trifluoromethylphenylboronic acid (0.27 g, 1.2 mmol) The crude was purified by flash chromatography on silica gel in hexane/ethyl acetate 2:1. A white solid was recovered (0.02 g) 13% yield. MS (ES) m/z 407.0 [M+1]⁺

### Example 24

### 9-[2-Chloro-5-(trifluoromethoxy)phenyl]-2-methoxy-6,7-dimethylimidazo[1,5-]pyrido[3,2-e]pyrazine

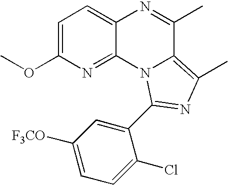

9-[2-Chloro-5-(trifluoromethoxy)phenyl]-2-methoxy-6,7-dimethylimidazo[1,5-a]pyrido[3,2-e]pyrazine was synthesized in a manner similar to Example 12, starting with 9-bromo-2-methoxy-6,7-dimethylimidazo[1,5-a]pyrido[3,2-e]pyrazine 5B (0.12 g, 0.39 mmol) and 2-chloro-5-trifluoromethoxyphenylboronic acid (0.29 g, 1.2 mmol) The crude was purified by flash chromatography on silica gel in hexane/ethyl acetate 2:1. A white solid was recovered (0.1 g) 61% yield. MS (ES) m/z 423.1 [M+1]⁺

### Example 25

### 4-Chloro-3-(2-methoxy-6,7-dimethylimidazo[1,5-a]pyrido[3,2-e]pyrazin-9-yl)benzonitrile

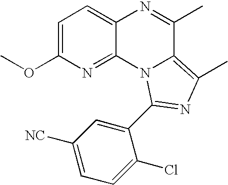

4-Chloro-3-(2-methoxy-6,7-dimethylimidazo[1,5-a]pyrido[3,2-e]pyrazin-9-yl)benzonitrile was synthesized in a manner similar to Example 12, starting with 9-bromo-2-methoxy-6,7-dimethylimidazo[1,5-a]pyrido[3,2-e]pyrazine 5B (0.12 g, 0.39 mmol) and 2-chloro-5-cyanophenylboronic acid (0.22 g, 1.2 mmol) The crude was purified by flash chromatography on silica gel in hexane/ethyl acetate 2:1. A white solid was recovered (0.09 g) 63% yield. MS (ES) m/z 364.1 [M+1]⁺

### Example 26

### 9-(2-Chloro-5-ethoxyphenyl)-2-methoxy-6,7-dimethylimidazo[1,5-a]pyrido[3,2-e]pyrazine

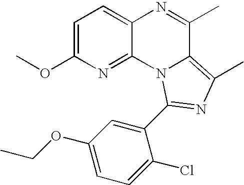

9-(2-Chloro-5-ethoxyphenyl)-2-methoxy-6,7-dimethylimidazo[1,5-a]pyrido[3,2-e]pyrazine was synthesized in a manner similar to Example 12, starting with 9-bromo-2-methoxy-6,7-dimethylimidazo[1,5-a]pyrido[3,2-e]pyrazine 5B (0.12 g, 0.39 mmol) and 2-chloro-5-ethoxyphenylboronic acid (0.24 g, 1.2 mmol) The crude was purified by flash chromatography on silica gel in hexane/ethyl acetate 2:1. A white solid was recovered (0.09 g) 60% yield. MS (ES) m/z 383.1 [M+1]⁺

### Example 27

### 2-Methoxy-6,7-dimethyl-9-pyrimidin-5-ylimidazo[1,5-a]pyrido[3,2-e]pyrazine

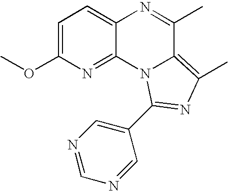

2-Methoxy-6,7-dimethyl-9-pyrimidin-5-ylimidazo[1,5-a]pyrido[3,2-e]pyrazine was synthesized in a manner similar to Example 12, starting with 9-bromo-2-methoxy-6,7-dimethylimidazo[1,5-a]pyrido[3,2-e]pyrazine 5B (0.12 g, 0.39 mmol) and 5-pyrimidineboronic acid (0.14 g, 1.2 mmol) The crude was purified by flash chromatography on silica gel in ethyl acetate. A white solid was recovered (0.02 g) 17% yield. MS (ES) m/z 307.1 [M+1]⁺

### Example 28

### 2-Methoxy-9-(6-methoxypyridin-3-yl)-6,7-dimethylimidazo[1,5-a]pyrido[3,2-e]pyrazine

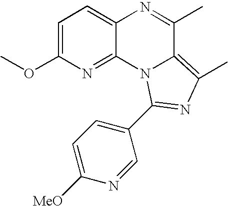

2-Methoxy-9-(6-methoxypyridin-3-yl)-6,7-dimethylimidazo[1,5-a]pyrido[3,2-e]pyrazine was synthesized in a manner similar to Example 12, starting with 9-bromo-2-methoxy-6,7-dimethylimidazo[1,5-a]pyrido[3,2-e]pyrazine 5B (0.12 g, 0.39 mmol) and 2-methoxy-5-pyridineboronic acid (0.18 g, 1.2 mmol) The crude was purified by flash chromatography on silica gel in ethyl acetate. A white solid was recovered (0.05 g) 38% yield. MS (ES) m/z 336.1 [M+1]⁺

### Example 29

### 2-Methoxy-9-(2-methoxypyridin-3-yl)-6,7-dimethylimidazo[1,5-a]pyrido[3,2-e]pyrazine

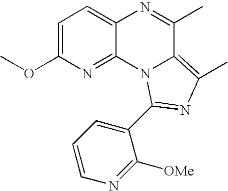

2-Methoxy-9-(2-methoxypyridin-3-yl)-6,7-dimethylimidazo[1,5-a]pyrido[3,2-e]pyrazine was synthesized in a manner similar to Example 12, starting with 9-bromo-2-methoxy-6,7-dimethylimidazo[1,5-a]pyrido[3,2-e]pyrazine 5B (0.12 g, 0.39 mmol) and 2-methoxy-3-pyridineboronic acid (0.18 g, 1.2 mmol) The crude was purified by flash chromatography on silica gel in ethyl acetate. A white solid was recovered (0.05 g) 38% yield. MS (ES) m/z 336.1 [M+1]⁺

### Example 30

### 2-Methoxy-9-(4-methoxypyridin-3-yl)-6,7-dimethylimidazo[1,5-a]pyrido[3,2-e]pyrazine

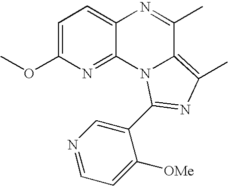

2-Methoxy-9-(4-methoxypyridin-3-yl)-6,7-dimethylimidazo[1,5-a]pyrido[3,2-e]pyrazine was synthesized in a manner similar to Example 12, starting with 9-bromo-2-methoxy-6,7-dimethylimidazo[1,5-a]pyrido[3,2-e]pyrazine 5B (0.12 g, 0.39 mmol) and 4-methoxy-3-pyridineboronic acid (0.18 g, 1.2 mmol) The crude was purified by flash chromatography on silica gel in ethyl acetate. A white solid was recovered (0.05 g) 38% yield. MS (ES) m/z 336.1 [M+1]⁺

### Example 31

### 9-(6-Fluoro-2-methylpyridin-3-yl)-2-methoxy-6,7-dimethylimidazo[1,5-a]pyrido[3,2-e]pyrazine

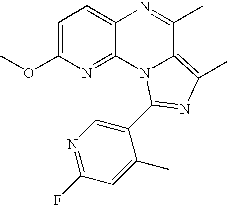

9-(6-Fluoro-2-methylpyridin-3-yl)-2-methoxy-6,7-dimethylimidazo[1,5-a]pyrido[3,2-e]pyrazine was synthesized in a manner similar to Example 12, starting with 9-bromo-2-methoxy-6,7-dimethylimidazo[1,5-a]pyrido[3,2-e]pyrazine 5B (0.12 g, 0.39 mmol) and 2-fluoro-4-methyl-5-pyridineboronic acid (0.18 g, 1.2 mmol) The crude was purified by flash chromatography on silica gel in ethyl acetate. A white solid was recovered (0.06 g) 45% yield. MS (ES) m/z 338.1 [M+1]⁺

### Example 32

### 2-Methoxy-6,7-dimethyl-9-(4-methylpyridin-3-yl)imidazo[1,5-a]pyrido[3,2-e]pyrazine

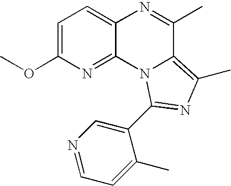

A mixture of bromide 5B (2.0 g, 6.53 mmol), 4-methylpyridine-3-boronic acid (1.79 g, 13.06 mmol), K₂CO₃ (2.70 g, 19.60 mmol) and Pd(PPh₃)₄ (150 mg, 0.1306 mmol) in a 250 M1 flask was vacuumed and flushed with nitrogen, followed by addition of p-dioxane (120 mL) and water (40 mL). The final mixture was stirred at 90° C. for 4 hours, then cooled to room temperature. The reaction was quenched with NH₄Cl solution, extracted with ethyl acetate. Combined organic layer was washed with brine, dried over magnesium sulfate. Column chromatography using 50% ethyl acetate in dichloromethane as eluent provided 2-methoxy-6,7-dimethyl-9-(4-methylpyridin-3-yl)imidazo[1,5-a]pyrido[3,2-e]pyrazine as an offwhite powder (1.68 g, 81% yield).

¹H NMR (400 MHz, DMSO) δ ppm 8.55 (s, 1H), 8.50 (m, 1H), 8.10 (d, 1H), 7.40 (m, 1H), 6.85 (d, 1H), 3.10 (s, 3H), 2.75 (s, 3H), 2.70 (s, 3H), 2.05 (s, 3H); EIMS 320.1 [M+H]+.

### Example 32

### Method C

The intermediate 5B 9-Bromo-2-methoxy-6,7-dimethylimidazo[1,5-a]pyrido[3,2-e]pyrazine was alternately prepared according to Scheme 5.

Example 32, 2-methoxy-6,7-dimethyl-9-(4-methylpyridin-3-yl)imidazo[1,5-a]pyrido[3,2-e]pyrazine, was prepared from 9-bromo-2-methoxy-6,7-dimethylimidazo[1,5-a]pyrido[3,2-e]pyrazine 5B according to Method B.

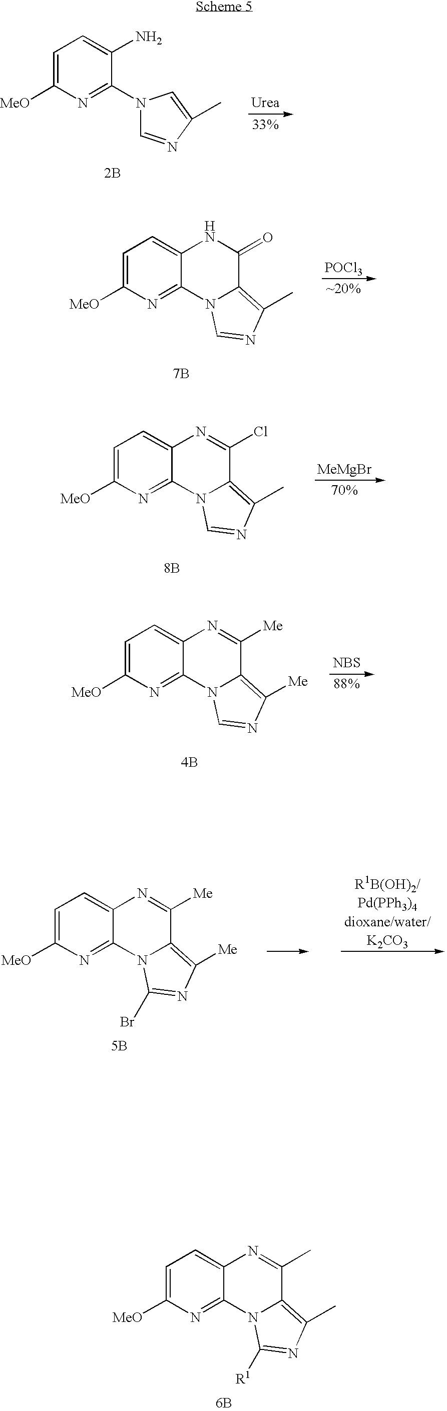

### 2-Methoxy-7-methylimidazo[1,5-a]pyrido[3,2-e]pyrazin-6(5H)-one (7B)

A mixture of substrate 2B (3.06 g, 15 mmol) and urea (12.8 g, 210 mmol) was heated to 160° C. for 4 hours, then 4 mL of glacial acetic acid was added and stirring was continued at 120° C. for additional 2 hours. The mixture was cooled to 70° C. and 80 mL water was added, stirred for 0.5 hour at room temperature and the mixture was filtered to provide 1.2 g (33% yield) of the desired product 7B. [M+H]⁺ 231.1 (ES).

### 6-Chloro-2-methoxy-7-methylimidazo[1,5-a]pyrido[3,2-e]pyrazine (8B)

A suspension of substrate 7B (920 mg, 4 mmol) in 20 mL POCl₃ was stirred at 110° C. for 5 hours. Major solvent was removed under vacuo and the residue was added slowly to iced methanol. Extraction with dichloromethane and condensation by rotavap provided 200 mg (20% yield) of product 8B as an offwhite solid. [M+H]⁺ 249.0 (ES).

### 2-Methoxy-6,7-dimethylimidazo[1,5-a]pyrido[3,2-e]pyrazine (4B)

To a suspension of substrate 8B (280 mg, 1.12 mmol) in 8 mL THF was added dropwise MeMgBr (3.0 M in Et₂O, 1.5 mL, 4.5 mmol) at 0° C. The resulting mixture was stirred at the same temperature for 20 minutes, then warmed to room temperature for 6 hours. The mixture was poured into iced-NH₄Cl solution slowly, stirred for 0.5 hour. Standard workup procedure followed by column purification provided 180 mg (70% yield) of product 4B as an offwhite solid. EIMS 229.0 [M+H]+.

### Example 33

### 9-(6-Fluoro-5-methylpyridin-3-yl)-2-methoxy-6,7-dimethylimidazo[1,5-a]pyrido[3,2-e]pyrazine

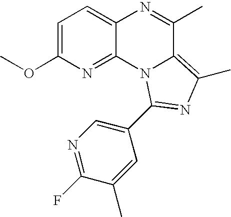

9-(6-Fluoro-5-methylpyridin-3-yl)-2-methoxy-6,7-dimethylimidazo[1,5-a]pyrido[3,2-e]pyrazine was synthesized in a manner similar to Example 12, starting with 9-bromo-2-methoxy-6,7-dimethylimidazo[1,5-a]pyrido[3,2-e]pyrazine 5B (0.12 g, 0.39 mmol) and 2-fluoro-3-methyl-5-pyridineboronic acid (0.18 g, 1.2 mmol) The crude was purified by flash chromatography on silica gel in ethyl acetate. A white solid was recovered (0.05 g) 37% yield. MS (ES) m/z 338.1 [M+1]⁺

Scheme 6 shows a synthetic method that was used in the preparation of examples 34-37.

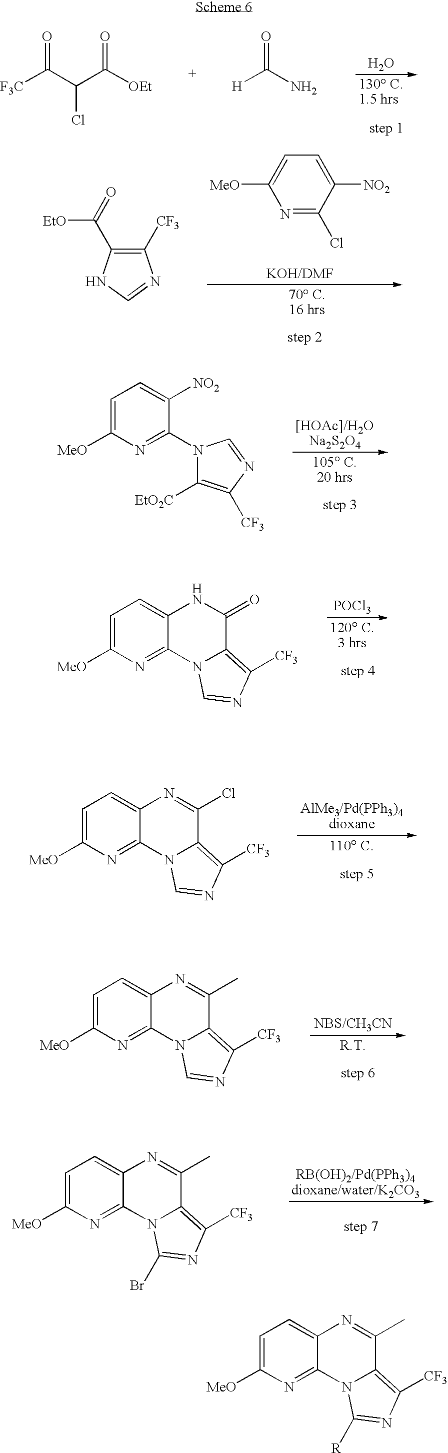

### Example 34

### 2-Methoxy-9-(4-methoxypyridin-3-yl)-6-methyl-7-(trifluoromethyl)imidazo[1,5-a]pyrido[3,2-e]pyrazine

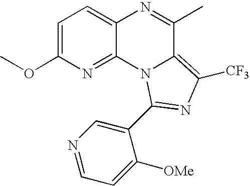

### Step 1

### 5-Trifluoromethyl-3H-imidazole-4-carboxylic Acid Ethyl Ester

Ethyl 2-chloro-4,4,4-trifluoroacetate (25 g, 0.114 mol) was combined with amidine (50 g, 1.1 mol) and water (5 mL). The reaction became warm and was heated to 130° C. for 1.5 hrs. The reaction was then cooled to room temperature and 100 mL of ice water added. The resulting solids were collected and washed with water then dried. 5.5 g, 23% of a brown solid was recovered as desired product. EIMS 209.05 [M+H]+.

### Step 2

### 3-(6-Methoxy-3-nitro-pyridin-2-yl)-5-trifluoromethyl-3H-imidazole-4-carboxylic Acid Ethyl Ester

5-Trifluoromethyl-3H-imidazole-4-carboxylic acid ethyl ester (Scheme 6, step 1) (5 g, 24 mmol) and 2-chloro-3-nitro-6-methoxypyridine (4.5 g, 24 mmol) were dissolved in DMF (60 mL). To this was added freshly powdered KOH (1.3 g, 24 mmol). The reaction was heated to 70° C. for 16 hrs then cooled to room temperature and diluted with water. The solution was then extracted with ethyl acetate 2×. The organic layers were combined and washed with water then brined and dried over MgSO₄. The solution was filtered and the solvent removed under reduced pressure. The crude was purified using flash chromatography on silica gel in hexane/ethyl acetate 2:1. A yellow oil (3.4 g, 39%) was recovered as desired product. EIMS 361.0 [M+H]+.

### Step 3

### 8-Methoxy-3-trifluoromethyl-5H-2,5,9,9b-tetraaza-cyclopenta[a]naphthalen-4-one

3-(6-Methoxy-3-nitro-pyridin-2-yl)-5-trifluoromethyl-3H-imidazole-4-carboxylic acid ethyl ester (Scheme 6, step 2) (2.9 g, 8.0 mmol) was dissolved in glacial acetic acid (45 mL). To this was added water (23 mL) followed by sodium hydrogensulfite (10 g, 80 mmol). The reaction was heated to 105° C. for 16 hrs. 2 g of the hydrogensulfite is then added every 2 hrs until starting material consumed, as indicated by TLC. The reaction was diluted with water and the solids filtered and collected. The solids were washed with water followed by a small amount of chloroform then dried. A grey/white solid (1.8 g, 79%) was recovered as desired product. EIMS 285.1 [M+H]+.

### Step 4

### 4-Chloro-8-methoxy-3-trifluoromethyl-2,5,9,9b-tetraaza-cyclopenta[a]naphthalene

8-Methoxy-3-trifluoromethyl-5H-2,5,9,9b-tetraaza-cyclopenta[a]naphthalen-4-one (Scheme 6, step 3) (1.0 g, 3.5 mmol) was suspended in POCl₃ (11 mL) and heated to 120° C. for 3 hrs. POCl₃ was removed under reduced pressure and the residue taken in water and neutralized with solid sodium bicarbonate. The resulting solids were filtered and collected then dried. A pale yellow solid (0.98 g, 99%) was recovered as desired product. EIMS 303.0 [M+H]+.

### Step 5

### 8-Methoxy-4-methyl-3-trifluoromethyl-2,5,9,9b-tetraaza-cyclopenta[a]naphthalene

4-Chloro-8-methoxy-3-trifluoromethyl-2,5,9,9b-tetraaza-cyclopenta[a]naphthalene (Scheme 6, step 4) (0.2 g, 0.66 mmol) was dissolved in dry dioxane (4 mL). To this was added Pd(PPh₃)₄ (0.012 g, 5% mol) followed by trimethylaluminum (2 M/toluene) (1.6 mL, 3.3 mmol). The reaction was heated to 110° C. for 2 hrs, then cooled in an ice bath. Dilute HCl (2 mL) was slowly added followed by dilute sodium hydroxide (4 mL). The reaction was extracted with ethyl acetate and the organic layers separated and combined. The combined extracts were washed with water then brined and dried over MgSO₄. The solution was filtered and the solvent removed under reduced pressure. The crude was purified using flash chromatography on silica gel in hexane/ethyl acetate 10:1. A white solid (0.12 g, 60%) was recovered as desired product. EIMS 283.0 [M+H]+.

### Step 6

### 1-Bromo-8-methoxy-4-methyl-3-trifluoromethyl-2,5,9,9b-tetraaza-cyclopenta[a]naphthalene

8-Methoxy-4-methyl-3-trifluoromethyl-2,5,9,9b-tetraazacyclopenta[a]naphthalene (Scheme 6, step 5, step 5) (0.12 g, 0.42 mmol) was suspended in acetonitrile (4 mL). N-bromosuccinimide (0.11 g, 0.6 mmol) was then added and the reaction protected from light and stirred for 16 hrs at room temperature. The reaction was then poured into aqueous sodium sulfite and extracted with ethyl acetate 2×. The organic layers were separated and combined then washed with water, brined and dried over MgSO₄. The solution was filtered and removed of solvent under reduced pressure. The crude was purified using flash chromatography on silica gel in hexane/ethyl acetate 10:2. A white solid (0.07 g, 45%) was recovered as desired product. EIMS 361.0 [M+H]+.

### Step 7

### 2-Methoxy-9-(4-methoxypyridin-3-yl)-6-methyl-7-(trifluoromethyl)imidazo[1,5-a]pyrido[3,2-e]pyrazine

2-Methoxy-9-(4-methoxypyridin-3-yl)-6-methyl-7-(trifluoromethyl)imidazo[1,5-a]pyrido[3,2-e]pyrazine was synthesized in a manner similar to Example 12, starting with 1-bromo-8-methoxy-4-methyl-3-trifluoromethyl-2,5,9,9b-tetraaza-cyclopenta[a]naphthalene (0.12 g, 0.33 mmol) (Scheme 6, step 6) and 4-methoxy-5-pyridineboronic acid (0.18 g, 1.0 mmol) The crude was purified by flash chromatography on silica gel in ethyl acetate. A white solid was recovered (0.04 g) 31% yield. MS (ES) m/z 390.1 [M+1]⁺

### Example 35

### 9-(2,5-Dichlorophenyl)-2-methoxy-6-methyl-7-(trifluoromethyl)imidazo[1,5-a]pyrido[3,2-e]pyrazine

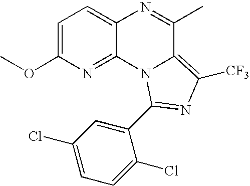

9-(2,5-Dichlorophenyl)-2-methoxy-6-methyl-7-(trifluoromethyl)imidazo[1,5-a]pyrido[3,2-e]pyrazine was synthesized in a manner similar to Example 12, starting with 1-bromo-8-methoxy-4-methyl-3-trifluoromethyl-2,5,9,9b-tetraaza-cyclopenta[a]naphthalene (0.12 g, 0.33 mmol) and 2,5-dichlorophenylboronic acid (0.22 g, 1.0 mmol) The crude was purified by flash chromatography on silica gel in hexane/ethyl acetate 2:1. A white solid was recovered (0.06 g) 45% yield. MS (ES) m/z 427.0 [M+1]⁺

### Example 36

### 4-Fluoro-3-[2-methoxy-6-methyl-7-(trifluoromethyl)imidazo[1,5-a]pyrido[3,2-e]pyrazin-9-yl]benzamide

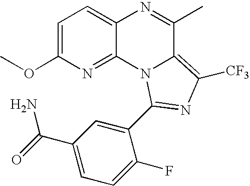

4-Fluoro-3-[2-methoxy-6-methyl-7-(trifluoromethyl)imidazo[1,5-a]pyrido[3,2-e]pyrazin-9-yl]benzamide was synthesized in a manner similar to Example 12, starting with 1-bromo-8-methoxy-4-methyl-3-trifluoromethyl-2,5,9,9b-tetraaza-cyclopenta[a]naphthalene (0.12 g, 0.33 mmol) and [5-carbamoyl-2-fluorophenyl]boronic acid (0.14 g, 0.66 mmol) The crude was purified by flash chromatography on silica gel in hexane/ethyl acetate 2:1. A white solid was recovered (0.08 g) 58% yield. MS (ES) m/z 420.1 [M+1]⁺

### Example 37

### 2-Methoxy-6-methyl-9-(2-methylphenyl)-7-(trifluoromethyl)imidazo[1,5-a]pyrido[3,2-e]pyrazine

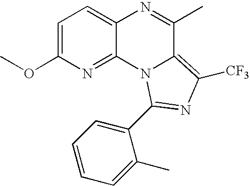

2-Methoxy-6-methyl-9-(2-methylphenyl)-7-(trifluoromethyl)imidazo[1,5-a]pyrido[3,2-e]pyrazine was synthesized in a manner similar to Example 12, starting with 1-bromo-8-methoxy-4-methyl-3-trifluoromethyl-2,5,9,9b-tetraaza-cyclopenta[a]naphthalene (Example 33, step 6) (0.12 g, 0.33 mmol) and 2-methylphenylboronic acid (0.16 g, 0.1 mmol) The crude was purified by flash chromatography on silica gel in hexane/ethyl acetate 2:1. A white solid was recovered (0.04 g) 32% yield. MS (ES) m/z 373.1 [M+1]⁺

Scheme 7 shows a synthetic method that was used in the preparation of Examples 38-39.

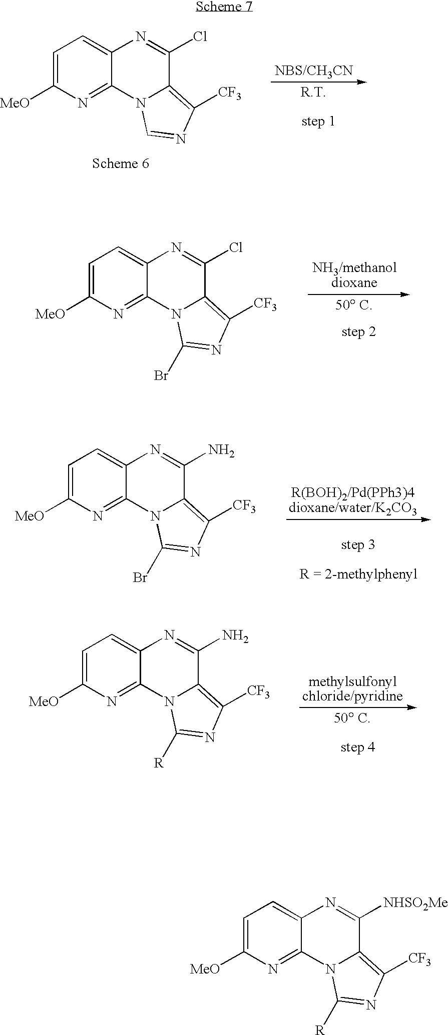

### Example 38

### 2-Methoxy-9-(2-methylphenyl)-7-(trifluoromethyl)imidazo[1,5-a]pyrido[3,2-e]pyrazin-6-amine

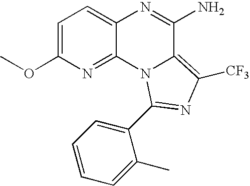

### Step 1

### 1-Bromo-4-chloro-8-methoxy-3-trifluoromethyl-2,5,9,9b-tetraaza-cyclopenta[a]naphthalene

4-Chloro-8-methoxy-3-trifluoromethyl-2,5,9,9b-tetraazacyclopenta[a]naphthalene (Scheme 6, Step 4) (0.10 g, 0.33 mmol) was suspended in acetonitrile (3 mL). N-bromosuccinimide (0.09 g, 0.5 mmol) was then added and the reaction protected from light and stirred for 16 hrs at room temperature. The reaction was then poured into aqueous sodium sulfite and extracted with ethyl acetate 2×. The organic layers were separated and combined then washed with water, brine and dried over MgSO₄. The solution was filtered and solvent was removed under reduced pressure. A white solid (0.1 g, 79%) was recovered as desired product. EIMS 380.0 [M+H]+.

### Step 2

### 1-Bromo-8-methoxy-3-trifluoromethyl-2,5,9,9b-tetraaza-cyclopenta[a]-naphthalen-4-ylamine

1-Bromo-4-chloro-8-methoxy-3-trifluoromethyl-2,5,9,9b-tetraaza-cyclopenta[a]naphthalene (Scheme 7, Step 1) (0.50 g, 1.3 mmol) was suspended in dioxane (3 mL). Ammonia 7M/methanol (3 mL) was then added and the reaction sealed and heated to 50° C. overnight. The reaction was let cool the filtered and the solids collected. A white solid (0.1 g, 20%) was recovered as desired product. EIMS 362.0 [M+H]+.

### Step 3

### 2-Methoxy-7-methyl-9-(2-methylphenyl)imidazo[1,5-a]pyrido[3,2-e]pyrazin-6-amine

2-Methoxy-7-methyl-9-(2-methylphenyl)imidazo[1,5-a]pyrido[3,2-e]pyrazin-6-amine was synthesized in a manner similar to Example 12, starting with 1-bromo-8-methoxy-3-trifluoromethyl-2,5,9,9b-tetraaza-cyclopenta[a]naphthalen-4-ylamine (0.10 g, 0.27 mmol) (scheme 7 step 2) and 2-methylphenylboronic acid (0.11 g, 0.8 mmol) The crude was purified by flash chromatography on silica gel in hexane/ethyl acetate 2:1. A white solid was recovered (0.06 g) 59% yield. MS (ES) m/z 374.1 [M+1]⁺

### Example 39

### N-[2-Methoxy-9-(2-methylphenyl)-7-(trifluoromethyl)imidazo[1,5-a]pyrido[3,2-e]pyrazin-6-yl]methanesulfonamide

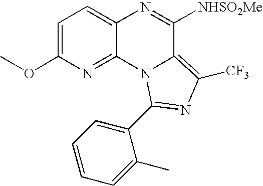

6-Chloro-2-methoxy-9-(2-methylphenyl)-7-(trifluoromethyl)imidazo[1,5-a]pyrido[3,2-e]pyrazine (Scheme 7, step 2) (0.14 g, 0.37 mmol) was suspended in pyridine (3 mL). To this was added methylsulfonyl chloride (0.09 mL, 1.1 mmol). The reaction was sealed and heated to 50° C. overnight. The reaction was diluted with water and extracted with ethyl acetate. The organic layer was separated and washed with dilute HCl then water, brined and dried over MgSO₄. After filtration, the solvent was removed under reduced pressure. The crude was purified by flash chromatography on silica gel in hexane/ethyl acetate 2:1. A yellow solid was recovered (0.01 g) 6% yield. MS (ES) m/z 452.0 [M+1].

Scheme 8 shows a synthetic method that was used in the preparation of Example 40.

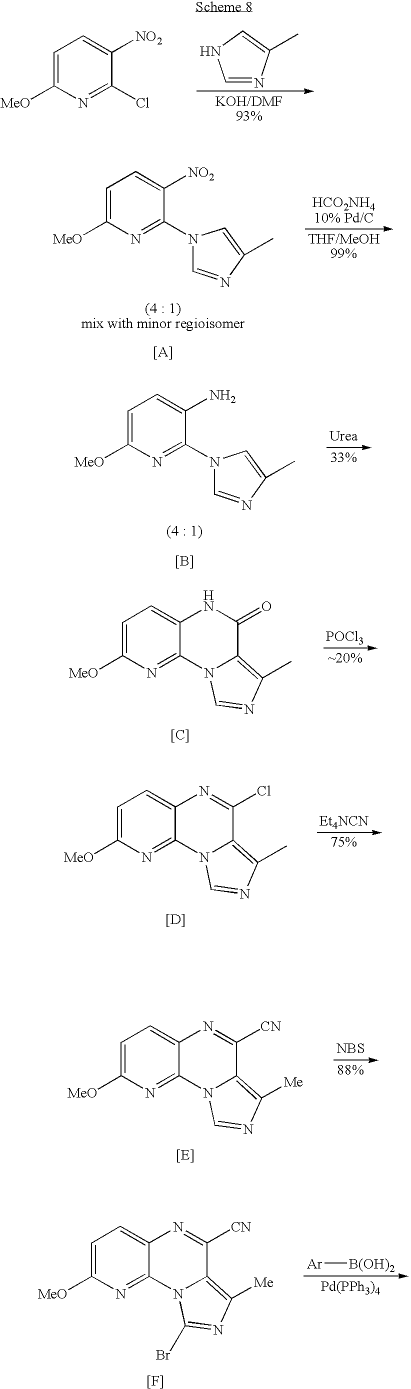

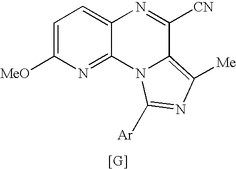

### Example 40

### 9-(2,5-Dichlorophenyl)-2-methoxy-7-methylimidazo[1,5-a]pyrido[3,2-e]pyrazine-6-carbonitrile

### Step 1

### 6-Methoxy-2-(4-methyl-1H-imidazol-1-yl)-3-nitropyridine

To a N,N-dimethylformamide (500 mL) solution of 4-methylimidazole (8.5 g, 103 mmol) was added freshly powdered KOH (6.72 g, 120 mmol) in two portions under N₂ at 0° C., followed by addition of 2-chloro-6-methoxy-3-nitropyridine (18.9 g, 100 mmol). The resulting solution was warmed to room temperature and stirred for 2 hours. Majority of solvent was removed under vacuum and the residue was diluted with water and extracted with ethyl acetate three times. The organic layer was combined and washed two more times with water to remove additional N,N-dimethylformamide and dried over magnesium sulfate. Solvent was evaporated under vacuum and the residue was purified by column (15-25% gradient eluent of ethyl acetate in dichloromethane) to provide a yellow oil (21.9 g, 93% yield) which becomes yellow solid after standing on bench. The ratio of two regioisomers was determined by NOE studies of intermediate B.

### Step 2

### 6-Methoxy-2-(4-methyl-1H-imidazol-1-yl)pyridin-3-amine

To a mixture of 6-methoxy-2-(4-methyl-1H-imidazol-1-yl)-3-nitropyridine (21.4 g, 91.5 mmol) and 10% Pd/C (5.12 g, 4.58 mmol) in a 1 L RB flask (connected with a condenser) was loaded 240 mL THF, followed by slow addition of 240 mL MeOH under N₂ with stirring. HCOONH₄ (34.75 g, 503.25 mmol) was added in two portions into the stirring mixture and the final mixture was stirred at room temperature for 10 min (gas released) and then warmed to 50° C. for 1 hr. The reaction was cooled to room temperature and filtered through celite. Solvent was evaporated under vacuum to dryness to provide clean product as an off-white powder (18.6 g, 99% yield). NMR indicated a 4:1 ratio mixture of two regioisomers with the major one as the desired regioisomer (confirmed by NOE studies).

### Step 3

### 2-Methoxy-7-methylimidazo[1,5-a]pyrido[3,2-e]pyrazin-6(5H)-one

A mixture of 6-methoxy-2-(4-methyl-1H-imidazol-1-yl)pyridin-3-amine (8.56 g, 41.8 mmol) and urea (35.8 g, 596.7 mmol) was heated to 140° C. for 10 min (solid melted) and then heated to 160° C. for 2 hours. Glacial acetic acid (6 mL) was added and stirred at 120° C. for additional 2 hours before cooling to 70° C. 80 mL water was added and the mixture was stirred at 70° C. for 30 min, then agitation was stopped. The precipitate was filtered and washed with water (2×25 mL) and dried in oven overnight to provide an off-white solid (3.2 g, 33% yield). NMR indicated some acyclic byproduct presented.

### Step 4

### 6-Chloro-2-methoxy-7-methylimidazo[1,5-a]pyrido[3,2-e]pyrazine

A suspension of 2-methoxy-7-methylimidazo[1,5-a]pyrido[3,2-e]pyrazin-6(5H)-one (1.6 g, 6.8 mmol) in 20 mL POCl₃ was stirred at 108° C. for 5 hours, then cooled to room temperature. POCl₃ was removed using toluene as co-solvent (2×50 mL) under vacuum. The residue was added water and dichloromethane. The mixture was agitated for 15 min. The aqueous phase was extracted with dichloromethane and the organic layer was washed with brine and dried over magnesium sulfate. Evaporation under vacuum to dryness provided clean product as an offwhite solid (280 mg, 20% yield).

### Step 5

### 2-Methoxy-7-methylimidazo[1,5-a]pyrido[3,2-e]pyrazine-6-carbonitrile

To a suspension of 6-chloro-2-methoxy-7-methylimidazo[1,5-a]pyrido[3,2-e]pyrazine (1.0 g, 4.27 mmol) in 10 mL DMSO was added tetramethylammonium cyanide (1.2 g, 4.17 mmol) under N₂ at 0° C. The resulting mixture was stirred at 75° C. for 2 hours. The mixture was poured into water extracted with chloroform. The organic phase was dried over magnesium sulfate. Evaporation under vacuum and purification by ISCO (20% ethyl acetate in dichloromethane) provided the product as a yellow solid (965 mg).

### Step 6

### 9-Bromo-2-methoxy-7-methylimidazo[1,5-a]pyrido[3,2-e]pyrazine-6-carbonitrile

To a mixture of 2-methoxy-7-methylimidazo[1,5-a]pyrido[3,2-e]pyrazine-6-carbonitrile (800 mg, 3.48 mmol) and NBS (619 mg, 3.48 mmol) was added anhydrous CH₃CN (20 mL) under N₂. The resulting solution was stirred in dark for 24 hours. The reaction was concentrated to dryness and the residue was dissolved in 30 mL ethyl acetate. The solution was washed twice with brine (2×30 mL), saturated Na₂SO₃ solution (20 mL) and brine (20 mL). All aqueous phase were combined and extracted with ethyl acetate (2×50 mL). The organic layers were combined and dried over magnesium sulfate. Evaporation under vacuum and purification by ISCO (20% ethyl acetate in dichloromethane) provided the product as a yellow solid (860 mg).

### Example 40

A flask containing the mixture of 9-bromo-2-methoxy-7-methylimidazo[1,5-a]pyrido[3,2-e]pyrazine-6-carbonitrile, 200 mg, 0.65 mmol), 2,5-dichlorophenylboronic acid (123 mg, 0.65 mmol), K₂CO₃ (267 mg, 1.94 mmol) and Pd(PPh₃)₄ (15 mg, 0.013 mmol) was vacuumed and refilled with nitrogen, followed by the addition of dioxane and H₂O (V/V 3:1). The final mixture was stirred at 90° C. for 1 hour and cooled to room temperature. The reaction was quenched with saturated NH₄Cl, extracted with ethyl acetate. Organic solution was washed with brine and dried over magnesium sulfate. Column chromatography using 20% ethyl acetate in dichloromethane as eluent provided the desired coupling product as a white solid (102 mg).

Scheme 9 shows a synthetic method that was used in the preparation of Examples 41-45.

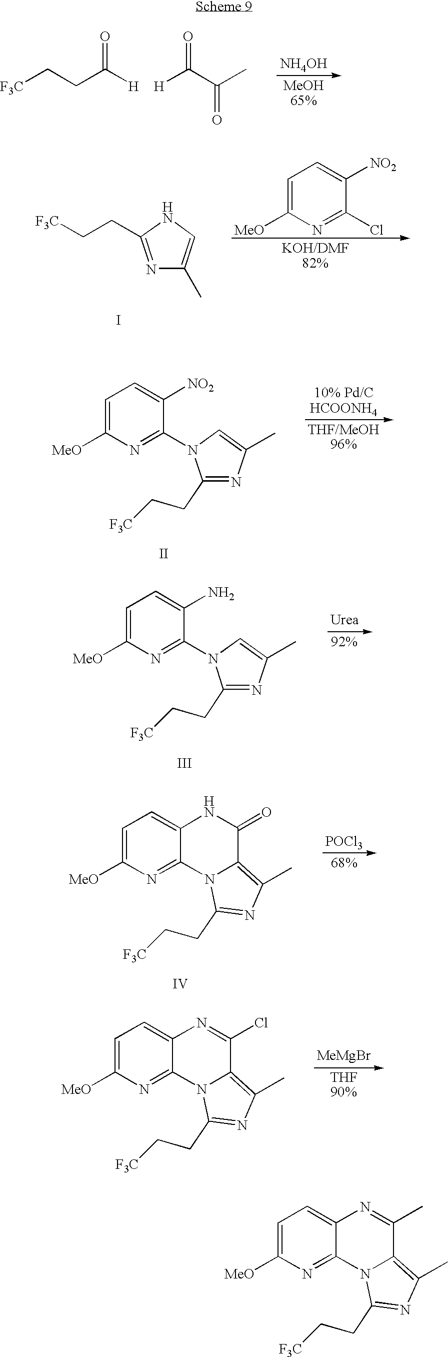

### Example 41

### 6-Chloro-2-methoxy-7-methyl-9-(3,3,3-trifluoropropyl)imidazo[1,5-a]pyrido[3,2-e]pyrazine

### Step 1

### 4-Methyl-2-(3,3,3-trifluoropropyl)-1H-imidazole

A solution of 4,4,4-trifluorobutanal (6.3 g, 50 mmol) in ethanol (25 mL) was treated with ammonium hydroxide (30%, 25 mL) and heated to 55° C. 2-Oxopropanal (40% in H₂O, 11.25 g, 62.5 mmol) was added dropwise and the resulting mixture was stirred at 60° C. overnight. The reaction mixture was poured into water, extracted with ethyl acetate and dried over magnesium sulfate. Column purification using ethyl acetate as eluent provided the product as a yellow solid (5.8 g, 65% yield). EIMS 179.1 [M+H]+.

### Step 2

### 6-Methoxy-2-(4-methyl-2-(3,3,3-trifluoropropyl)-1H-imidazol-1-yl)-3-nitropyridine

To a solution of 4-methyl-2-(3,3,3-trifluoropropyl)-1H-imidazole (1 g, 5.6 mmol) in DMF (25 mL) was added freshly powdered KOH (366 mg, 6.54 mmol) at 0° C. under nitrogen, followed by addition of 2-chloro-6-methoxy-3-nitropyridine (1.03 g, 5.45 mmol). The resulting brown solution was stirred at room temperature for 2 hours and then poured into ice-water. The mixture was extracted with ethyl acetate, washed with brine and dried over MgSO₄. Column purification using 10-25% ethyl acetate in hexane as eluent provided the product as a yellow powder (1.46 g, 82% yield). EIMS 331.0 [M+H]+.

### Step 3

### 6-Methoxy-2-(4-methyl-2-(3,3,3-trifluoropropyl)-1H-imidazol-1-yl)pyridin-3-amine

To a mixture of 6-methoxy-2-(4-methyl-2-(3,3,3-trifluoropropyl)-1H-imidazol-1-yl)-3-nitropyridine (8.1 g, 24.5 mmol) and 10% Pd/C (1.38 g, 1.22 mmol) in a 250 mL RB flask (connected with a condenser) was loaded 80 mL THF, followed by slow addition of 80 mL MeOH with stirring. HCOONH₄ (9.31 g, 134.75 mmol) was added in three portions into the stirring mixture and the final mixture was stirred at room temperature for 10 min (gas released) and then warmed to 50° C. for 1 hr. The reaction was cooled to room temperature and filtered through celite. Solvent was evaporated by rotovap and the residue was partitioned between water (˜100 mL) and ethyl acetate (˜150 mL). Aqueous phase was extracted with ethyl acetate (3×50 mL). The combined organic phase was dried over MgSO₄. Solvent was evaporated to provide clean product as an offwhite powder (7.22 g, 98% yield). EIMS 301.0 [M+H]+.

### Step 4

### 2-Methoxy-7-methyl-9-(3,3,3-trifluoropropyl)imidazo[1,5-a]pyrido[3,2-e]pyrazin-6(5H)-one

A mixture of 6-methoxy-2-(4-methyl-2-(3,3,3-trifluoropropyl)-1H-imidazol-1-yl)pyridin-3-amine (6.87 g, 22.9 mmol) and urea (19.8 g, 330 mmol) was heated to 160° C. for 4 hours, then 5 mL of glacial acetic acid was added. The mixture was stirred at 120° C. for additional 2 hours, cooled to 70° C. and added 100 mL water. Stirring was continued for 30 minutes and the reaction was cooled to room temperature overnight. The precipitate was collected and washed with water (2×25 mL), dried in oven for 2 hours. The product was obtained as an off-white solid (6.9 g, 92% yield). EIMS 327.1 [M+H]+.

### Step 5

### 6-Chloro-2-methoxy-7-methyl-9-(3,3,3-trifluoropropyl)imidazo[1,5-a]pyrido[3,2-e]pyrazine

A mixture of 2-methoxy-7-methyl-9-(3,3,3-trifluoropropyl)imidazo[1,5-a]pyrido[3,2-e]pyrazin-6(5H)-one (6.9 g, 21.1 mmol) in 40 mL of POCl₃ was refluxed at 120° C. for 4 hours, then cooled to room temperature. The solvent was removed under vacuum. Cold water was added very slowly, followed by addition of dichloromethane. The mixture was stirred for 15 minutes and extracted with dichloromethane, dried over magnesium sulfate. Column purification using 10-20% ethyl acetate in dichloromethane as eluent provided the product as an off-white powder (4.93 g, 68% yield). EIMS 345.0 [M+H]+.

### Example 42

### 2-Methoxy-6,7-dimethyl-9-(3,3,3-trifluoropropyl)imidazo[1,5-a]pyrido[3,2-e]pyrazine

To a solution of 6-Chloro-2-methoxy-7-methyl-9-(3,3,3-trifluoropropyl)imidazo[1,5-a]pyrido[3,2-e]pyrazine (172 mg, 0.5 mmol) in tetrahydrofuran (3 mL) was added MeMgBr (3.0 M in ethyl ether, 0.6 mL, 2.0 mmol) dropwise at 0° C. The resulting solution was stirred at room temperature overnight. The mixture was cooled to 0° C. and quenched with saturated NH₄Cl aqueous solution very carefully. Extraction with dichloromethane and column purification using 50% ethyl acetate in hexane as eluent provided the product as a yellow powder (145 mg, 90% yield). EIMS 325.0 [M+H]+.

### Example 43

### 6-Azetidin-1-yl-2-methoxy-7-methyl-9-(3,3,3-trifluoropropyl)imidazo[1,5-a]pyrido[3,2-e]pyrazine

To a suspension of 6-Chloro-2-methoxy-7-methyl-9-(3,3,3-trifluoropropyl)imidazo[1,5-a]pyrido[3,2-e]pyrazine (172 mg, 0.5 mmol) in 1 mL of ethanol was added azetidine (0.1 mL, 1.5 mmol) at room temperature. The resulting mixture was stirred under microwave (150° C.) for 10 minutes. Solvent was removed under vacuum. Column purification using 20% ethyl acetate in dichloromethane as eluent provided the product as a white powder (146 mg, 80% yield). EIMS 366.1 [M+H]+.

### Example 44

### 2-Methoxy-7-methyl-9-(3,3,3-trifluoropropyl)imidazo[1,5-a]pyrido[3,2-e]pyrazin-6-amine

To a mixture of 6-Chloro-2-methoxy-7-methyl-9-(3,3,3-trifluoropropyl)imidazo[1,5-a]pyrido[3,2-e]pyrazine (1.78 g, 5.1 mmol) in ethanol (12 mL) was added ammonium in methanol (7 N, 12 mL) quickly. The resulting mixture was stirred in a sealed tube at 100° C. for 3 days, cooled to room temperature. The precipitate was collected to provide clean product as an off-white powder (1.33 g, 80% yield). EIMS 326.1 [M+H]+.

### Example 45

### N-[2-Methoxy-7-methyl-9-(3,3,3-trifluoropropyl)imidazo[1,5-a]pyrido[3,2-e]pyrazin-6-yl]methanesulfonamide

To a mixture of 2-Methoxy-7-methyl-9-(3,3,3-trifluoropropyl)imidazo[1,5-a]pyrido[3,2-e]pyrazin-6-amine (1.31 g, 4.0 mmol) in pyridine (30 mL) was added MeSO₂Cl (1.0 mL, 12.0 mmol). The resulting mixture was stirred at 40° C. for 2 days. Pyridine was removed under vacuum. The residue was dissolved in dichloromethane and water, extracted with dichloromethane, dried over magnesium sulfate. Column purification using 10-25% ethyl acetate in dichloromethane as eluent provided the desired product as an off-white powder (700 mg, 44%). EIMS 404.1 [M+H]+.

### Example 46

### 6,7-Dimethyl-9-propylimidazo[1,5-a]pyrido[3,2-e]pyrazin-2(1H)-one

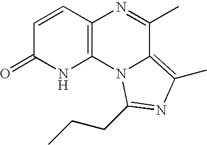

To a solution of 2-methoxy-6,7-dimethyl-9-propylimidazo[1,5-a]pyrido[3,2-e]pyrazine (270 mg, 1 mmol) in 4 mL of dichloromethane was added BBr₃ (0.48 mL, 5 mmol) dropwise at 0° C., then slowly warmed to 40° C. for 1 hour and refluxed at 50° C. for another 1 hour. The reaction was added K₂CO₃ aqueous solution at 0° C. Solvent was removed under vacuum and the residue was purified by column using 5% methanol in dichloromethane as eluent to provide the product as a white powder (100 mg, 40% yield). EIMS 257.1 [M+H]+.

### General Experimental for Suzuki Coupling

A vial or RB flask containing the mixture of bromide 5B (1 equivalent), aryl boronic acid (1.5˜2 equivalent), K₂CO₃ (3 equivalent) and Pd(PPh₃)₄ (0.05 equivalent) was vacuumed and refilled with nitrogen, followed by the addition of dioxane and H₂O (concentration=0.05 molar, V/V 3:1). The final mixture was stirred at 90° C. for 1˜4 hours and cooled to room temperature. The reaction was quenched with saturated NH₄Cl, extracted with ethyl acetate. Organic solution was washed with brine and dried over magnesium sulfate. Column chromatography using 20-50% ethyl acetate in dichloromethane as eluent provided the desired coupling product.

### Example 47

### 9-(2,5-Dichlorophenyl)-2-methoxy-6,7-dimethylimidazo[1,5-a]pyrido[3,2-e]pyrazine

Following the general Suzuki coupling procedure, reaction of bromide 5B (800 mg, 2.61 mmol), 2,5-dichlorophenylboronic acid (600 mg, 3.14 mmol), K₂CO₃ (1.08 g, 7.83 mmol) and Pd(PPh₃)₄ (60 mg, 0.0522 mmol) provided the coupling product as a white powder (770 mg, 80% yield). EIMS 372.8 [M+H]+.

### Example 48

### 9-(3-Chlorophenyl)-2-methoxy-6,7-dimethylimidazo[1,5-a]pyrido[3,2-e]pyrazine

Following the general Suzuki coupling procedure, reaction of bromide 5B (60 mg, 0.19 mmol), 3-chlorophenylboronic acid (33.8 mg, 0.21 mmol), K₂CO₃ (80 mg, 0.57 mmol) and Pd(PPh₃)₄ (11.6 mg, 0.01 mmol) provided the coupling product as a white powder (41 mg, 64% yield). EIMS 338.9 [M+H]+.

### Example 49

### 2-(2-Methoxy-6,7-dimethylimidazo[1,5-a]pyrido[3,2-e]pyrazin-9-yl)benzamide

Following the general Suzuki coupling procedure, reaction of bromide 5B (120 mg, 0.39 mmol), 2-cyanophenylboronic acid (63.2 mg, 0.43 mmol), K₂CO₃ (162.8 mg, 1.18 mmol) and Pd(PPh₃)₄ (22.6 mg, 0.0196 mmol) provided the hydrolyzed product as a yellow powder (45 mg, 33% yield). EIMS 348.1 [M+H]+.

### Example 50

### 2-Methoxy-6,7-dimethyl-9-(2-methylphenyl)imidazo[1,5-a]pyrido[3,2-e]pyrazine

Following the general Suzuki coupling procedure, reaction of bromide 5B (120 mg, 0.39 mmol), 2-methylphenylboronic acid (58.5 mg, 0.43 mmol), K₂CO₃ (162.8 mg, 1.18 mmol) and Pd(PPh₃)₄ (22.6 mg, 0.0196 mmol) provided the coupling product as a yellow powder (122 mg, 98% yield). EIMS 319.1 [M+H]+.

### Example 51

### 2-Methoxy-6,7-dimethyl-9-[2-(trifluoromethyl)phenyl]imidazo[1,5-a]pyrido[3,2-e]pyrazine

Following the general Suzuki coupling procedure, reaction of bromide 5B (120 mg, 0.39 mmol), 2-trifluoromethylphenylboronic acid (81.7 mg, 0.43 mmol), K₂CO₃ (162.8 mg, 1.18 mmol) and Pd(PPh₃)₄ (22.6 mg, 0.0196 mmol) provided the coupling product as an off-white powder (142 mg, 98% yield). EIMS 373.1 [M+H]+.

### Example 52

### 2-Methoxy-9-(2-methoxyphenyl)-6,7-dimethylimidazo[1,5-a]pyrido[3,2-e]pyrazine

Following the general Suzuki coupling procedure, reaction of bromide 5B (120 mg, 0.39 mmol), 2-methoxyphenylboronic acid (65.3 mg, 0.43 mmol), K₂CO₃ (162.8 mg, 1.18 mmol) and Pd(PPh₃)₄ (22.6 mg, 0.0196 mmol) provided the coupling product as an off-white powder (130 mg, 100% yield). EIMS 335.1 [M+H]+.

### Example 53

### 2-Methoxy-6,7-dimethyl-9-[2-(trifluoromethoxy)phenyl]imidazo[1,5-a]pyrido[3,2-e]pyrazine

Following the general Suzuki coupling procedure, reaction of bromide 5B (120 mg, 0.39 mmol), 2-trifluoromethoxyphenylboronic acid (88.5 mg, 0.43 mmol), K₂CO₃ (162.8 mg, 1.18 mmol) and Pd(PPh₃)₄ (22.6 mg, 0.0196 mmol) provided the coupling product as an off-white powder (143 mg, 95% yield). EIMS 389.1 [M+H]+.

### Example 54

### 9-(2-Isopropoxyphenyl)-2-methoxy-6,7-dimethylimidazo[1,5-a]pyrido[3,2-e]pyrazine

Following the general Suzuki coupling procedure, reaction of bromide 5B (120 mg, 0.39 mmol), 2-isopropoxyphenylboronic acid (77.4 mg, 0.43 mmol), K₂CO₃ (162.8 mg, 1.18 mmol) and Pd(PPh₃)₄ (22.6 mg, 0.0196 mmol) provided the coupling product as an off-white powder (140 mg, 99% yield). EIMS 363.2 [M+H]+.

### Example 55

### 2-Methoxy-9-(4-methoxyphenyl)-6,7-dimethylimidazo[1,5-a]pyrido[3,2-e]pyrazine

Following the general Suzuki coupling procedure, reaction of bromide 5B (100 mg, 0.32 mmol), 4-methoxyphenylboronic acid (54.7 mg, 0.36 mmol), K₂CO₃ (135.2 mg, 0.98 mmol) and Pd(PPh₃)₄ (18.5 mg, 0.016 mmol) provided the coupling product as an off-white powder (82 mg, 77% yield). EIMS 335.2 [M+H]+.

### Example 56

### 2-Methoxy-6,7-dimethyl-9-(3-thienyl)imidazo[1,5-a]pyrido[3,2-e]pyrazine

Following the general Suzuki coupling procedure, reaction of bromide 5B (120 mg, 0.39 mmol), 3-thienylboronic acid (62 mg, 0.48 mmol), K₂CO₃ (162.8 mg, 1.18 mmol) and Pd(PPh₃)₄ (22.6 mg, 0.0196 mmol) provided the coupling product as an off-white powder (90 mg, 75% yield). EIMS 311.1 [M+H]+.

### Example 57

### 2-Methoxy-6,7-dimethyl-9-(3-methyl-2-thienyl)imidazo[1,5-a]pyrido[3,2-e]pyrazine

Following the general Suzuki coupling procedure, reaction of bromide 5B (120 mg, 0.39 mmol), 3-methyl-2-thienylboronic acid (68 mg, 0.48 mmol), K₂CO₃ (162.8 mg, 1.18 mmol) and Pd(PPh₃)₄ (22.6 mg, 0.0196 mmol) provided the coupling product as an off-white powder (74 mg, 59% yield). EIMS 325.1 [M+H]+.

### Example 58

### 9-(3-Furyl)-2-methoxy-6,7-dimethylimidazo[1,5-a]pyrido[3,2-e]pyrazine

Following the general Suzuki coupling procedure, reaction of bromide 5B (120 mg, 0.39 mmol), 3-furylboronic acid (54 mg, 0.48 mmol), K₂CO₃ (162.8 mg, 1.18 mmol) and Pd(PPh₃)₄ (22.6 mg, 0.0196 mmol) provided the coupling product as an off-white powder (68 mg, 60% yield). EIMS 295.1 [M+H]+.

### Example 59

### 2-Methoxy-6,7-dimethyl-9-(4-methylphenyl)imidazo[1,5-a]pyrido[3,2-e]pyrazine

Following the general Suzuki coupling procedure, reaction of bromide 5B (100 mg, 0.32 mmol), 4-methylphenylboronic acid (49 mg, 0.36 mmol), K₂CO₃ (135.2 mg, 0.98 mmol) and Pd(PPh₃)₄ (18.5 mg, 0.016 mmol) provided the coupling product as an off-white powder (77 mg, 75% yield). EIMS 319.2 [M+H]+.

### Example 60

### 9-(2-Furyl)-2-methoxy-6,7-dimethylimidazo[1,5-a]pyrido[3,2-e]pyrazine

Following the general Suzuki coupling procedure, reaction of bromide 5B (120 mg, 0.39 mmol), 2-furylboronic acid (54 mg, 0.48 mmol), K₂CO₃ (162.8 mg, 1.18 mmol) and Pd(PPh₃)₄ (22.6 mg, 0.0196 mmol) provided the coupling product as an off-white powder (89 mg, 77% yield). EIMS 295.1 [M+H]+.

### Example 61

### 9-(3,5-Dimethylisoxazol-4-yl)-2-methoxy-6,7-dimethylimidazo[1,5-a]pyrido[3,2-e]pyrazine

Following the general Suzuki coupling procedure, reaction of bromide 5B (120 mg, 0.39 mmol), 3,5-dimethylisoxazolboronic acid (68 mg, 0.48 mmol), K₂CO₃ (162.8 mg, 1.18 mmol) and Pd(PPh₃)₄ (22.6 mg, 0.0196 mmol) provided the coupling product as an off-white powder (30 mg, 24% yield). EIMS 324.1 [M+H]+.

### Example 62

### 2-Methoxy-9-(3-methoxyphenyl)-6,7-dimethylimidazo[1,5-a]pyrido[3,2-e]pyrazine

Following the general Suzuki coupling procedure, reaction of bromide 5B (100 mg, 0.32 mmol), 3-methoxyphenylboronic acid (54.7 mg, 0.36 mmol), K₂CO₃ (135.2 mg, 0.98 mmol) and Pd(PPh₃)₄ (18.5 mg, 0.016 mmol) provided the coupling product as a light yellow powder (59 mg, 55% yield). EIMS 335.2 [M+H]+.

### Example 63

### 2-Methoxy-6,7-dimethyl-9-[3-(trifluoromethoxy)phenyl]imidazo[1,5-a]pyrido[3,2-e]pyrazine

Following the general Suzuki coupling procedure, reaction of bromide 5B (100 mg, 0.32 mmol), 3-trifluoromethoxyphenylboronic acid (74.2 mg, 0.36 mmol), K₂CO₃ (135.2 mg, 0.98 mmol) and Pd(PPh₃)₄ (18.5 mg, 0.016 mmol) provided the coupling product as an off-white powder (100 mg, 80% yield). EIMS 389.2 [M+H]+.

### Example 64

### 2-Methoxy-6,7-dimethyl-9-[4-(trifluoromethoxy)phenyl]imidazo[1,5-a]pyrido[3,2-e]pyrazine

Following the general Suzuki coupling procedure, reaction of bromide 5B (100 mg, 0.32 mmol), 4-trifluoromethoxyphenylboronic acid (74.2 mg, 0.36 mmol), K₂CO₃ (135.2 mg, 0.98 mmol) and Pd(PPh₃)₄ (18.5 mg, 0.016 mmol) provided the coupling product as a light yellow powder (82 mg, 66% yield). EIMS 389.2 [M+H]+.

### Example 65

### 2-Methoxy-6,7-dimethyl-9-(3-methylphenyl)imidazo[1,5-a]pyrido[3,2-e]pyrazine

Following the general Suzuki coupling procedure, reaction of bromide 5B (80 mg, 0.26 mmol), 3-methylphenylboronic acid (40 mg, 0.29 mmol), K₂CO₃ (108 mg, 0.78 mmol) and Pd(PPh₃)₄ (6 mg, 0.0052 mmol) provided the coupling product as an off-white powder (52 mg, 63% yield). EIMS 319.1 [M+H]+.

### Example 66

### 2-Methoxy-6,7-dimethyl-9-[3-(trifluoromethyl)phenyl]imidazo[1,5-a]pyrido[3,2-e]pyrazine

Following the general Suzuki coupling procedure, reaction of bromide 5B (80 mg, 0.26 mmol), 3-trifluoromethylphenylboronic acid (57 mg, 0.29 mmol), K₂CO₃ (108 mg, 0.78 mmol) and Pd(PPh₃)₄ (6 mg, 0.0052 mmol) provided the coupling product as an off-white powder (76 mg, 79% yield). EIMS 373.1 [M+H]+.

### Example 67

### 2-Methoxy-6,7-dimethyl-9-[4-(trifluoromethyl)phenyl]imidazo[1,5-a]pyrido[3,2-e]pyrazine

Following the general Suzuki coupling procedure, reaction of bromide 5B (80 mg, 0.26 mmol), 4-trifluoromethylphenylboronic acid (57 mg, 0.29 mmol), K₂CO₃ (108 mg, 0.78 mmol) and Pd(PPh₃)₄ (6 mg, 0.0052 mmol) provided the coupling product as an off-white powder (54 mg, 56% yield). EIMS 373.1 [M+H]+.

### Example 68

### 2-Methoxy-6,7-dimethyl-9-(2-thienyl)imidazo[1,5-a]pyrido[3,2-e]pyrazine

Following the general Suzuki coupling procedure, reaction of bromide 5B (100 mg, 0.32 mmol), 2-thienylboronic acid (46 mg, 0.36 mmol), K₂CO₃ (135.2 mg, 0.98 mmol) and Pd(PPh₃)₄ (7.5 mg, 0.0064 mmol) provided the coupling product as an off-white powder (58 mg, 58% yield). EIMS 311.1 [M+H]+.

### Example 69

### 2-Methoxy-6,7-dimethyl-9-(4-methyl-3-thienyl)imidazo[1,5-a]pyrido[3,2-e]pyrazine

Following the general Suzuki coupling procedure, reaction of bromide 5B (100 mg, 0.32 mmol), 4-methyl-3-thienylboronic acid (52 mg, 0.36 mmol), K₂CO₃ (135.2 mg, 0.98 mmol) and Pd(PPh₃)₄ (7.5 mg, 0.0064 mmol) provided the coupling product as an off-white powder (68 mg, 66% yield). EIMS 325.1 [M+H]+.

### Example 70

### 9-Biphenyl-2-yl-2-methoxy-6,7-dimethylimidazo[1,5-a]pyrido[3,2-e]pyrazine

Following the general Suzuki coupling procedure, reaction of bromide 5B (100 mg, 0.32 mmol), biphenyl-2-ylboronic acid (72 mg, 0.36 mmol), K₂CO₃ (135.2 mg, 0.98 mmol) and Pd(PPh₃)₄ (7.5 mg, 0.0064 mmol) provided the coupling product as an off-white powder (74 mg, 61% yield). EIMS 381.1 [M+H]+.

### Example 71

### 9-Biphenyl-3-yl-2-methoxy-6,7-dimethylimidazo[1,5-a]pyrido[3,2-e]pyrazine

Following the general Suzuki coupling procedure, reaction of bromide 5B (100 mg, 0.32 mmol), biphenyl-3-ylboronic acid (72 mg, 0.36 mmol), K₂CO₃ (135.2 mg, 0.98 mmol) and Pd(PPh₃)₄ (7.5 mg, 0.0064 mmol) provided the coupling product as an off-white powder (79 mg, 65% yield). EIMS 381.1 [M+H]+.

### Example 72

### 9-Biphenyl-4-yl-2-methoxy-6,7-dimethylimidazo[1,5-a]pyrido[3,2-e]pyrazine

Following the general Suzuki coupling procedure, reaction of bromide 5B (100 mg, 0.32 mmol), biphenyl-4-ylboronic acid (72 mg, 0.36 mmol), K₂CO₃ (135.2 mg, 0.98 mmol) and Pd(PPh₃)₄ (7.5 mg, 0.0064 mmol) provided the coupling product as an off-white powder (73 mg, 60% yield). EIMS 381.1 [M+H]+.

### Example 73

### 3-(2-Methoxy-6,7-dimethylimidazo[1,5-a]pyrido[3,2-e]pyrazin-9-yl)benzonitrile

Following the general Suzuki coupling procedure, reaction of bromide 5B (80 mg, 0.26 mmol), 3-cyanophenylboronic acid (48 mg, 0.32 mmol), K₂CO₃ (108 mg, 0.78 mmol) and Pd(PPh₃)₄ (6 mg, 0.0052 mmol) provided the coupling product as an off-white powder (55 mg, 65% yield). EIMS 330.1 [M+H]+.

### Example 74

### 4-(2-Methoxy-6,7-dimethylimidazo[1,5-a]pyrido[3,2-e]pyrazin-9-yl)benzonitrile

Following the general Suzuki coupling procedure, reaction of bromide 5B (80 mg, 0.26 mmol), 4-cyanophenylboronic acid (48 mg, 0.32 mmol), K₂CO₃ (108 mg, 0.78 mmol) and Pd(PPh₃)₄ (6 mg, 0.0052 mmol) provided the coupling product as an off-white powder (62 mg, 72% yield). EIMS 330.1 [M+H]+.

### Example 75

### 2-Methoxy-6,7-dimethyl-9-(phenylethynyl)imidazo[1,5-a]pyrido[3,2-e]pyrazine

To a pre-dried flask was charged with bromide 5B (80 mg, 0.26 mmol), DMF (3 mL), Et₃N (0.11 mL, 0.78 mmol) and phenylacetylene (33 mg, 0.32 mmol) under nitrogen, followed by addition of Pd(PPh₃)₂Cl₂ (3.6 mg, 0.0052 mmol) and CuI (2 mg, 0.0104 mmol). The mixture was stirred at 85° C. for 2 hours and cooled to room temperature. The reaction was poured into saturated NH₄Cl aqueous solution, extracted with ethyl acetate and dried over magnesium sulfate. Column purification using 20-50% ethyl acetate in dichloromethane as eluent provided the coupling product as a light yellow powder (82 mg, 96% yield). EIMS 329.1 [M+H]+.

### Example 76

### 9-[(4-Fluorophenyl)ethynyl]-2-methoxy-6,7-dimethylimidazo[1,5-a]pyrido[3,2-e]pyrazine

Following the procedure of preparing Example 75, reaction of bromide 5B 80 mg, 0.26 mmol), DMF (3 mL), Et₃N (0.11 mL, 0.78 mmol), 4-fluorophenylacetylene (38.4 mg, 0.32 mmol), Pd(PPh₃)₂Cl₂ (3.6 mg, 0.0052 mmol) and CuI (2 mg, 0.0104 mmol) provided the coupling product as a light yellow powder (40 mg, 44% yield). EIMS 347.1 [M+H]+.

### Example 77

### 2-Methoxy-9-[(4-methoxyphenyl)ethynyl]-6,7-dimethylimidazo[1,5-a]pyrido[3,2-e]pyrazine

Following the procedure of preparing Example 75, reaction of bromide 5B (80 mg, 0.26 mmol), DMF (3 mL), Et₃N (0.11 mL, 0.78 mmol), 4-methoxyphenylacetylene (42.2 mg, 0.32 mmol), Pd(PPh₃)₂Cl₂ (3.6 mg, 0.0052 mmol) and CuI (2 mg, 0.0104 mmol) provided the coupling product as a light yellow powder (32 mg, 34% yield). EIMS 359.1 [M+H]+.

### Example 78

### 9-(2-Chloro-5-methylphenyl)-2-methoxy-6,7-dimethylimidazo[1,5-a]pyrido[3,2-e]pyrazine

Following the general Suzuki coupling procedure, reaction of bromide 5B (60 mg, 0.196 mmol), 2-chloro-5-methylphenylboronic acid (40 mg, 0.235 mmol), K₂CO₃ (80 mg, 0.588 mmol) and Pd(PPh₃)₄ (5 mg, 0.0039 mmol) provided the coupling product as a white powder (63 mg, 92% yield). EIMS 353.1 [M+H]+.

### Example 79

### 9-(5-Chloro-2-methylphenyl)-2-methoxy-6,7-dimethylimidazo[1,5-a]pyrido[3,2-e]pyrazine

Following the general Suzuki coupling procedure, reaction of bromide 5B (80 mg, 0.26 mmol), 5-chloro-2-methylphenylboronic acid (53 mg, 0.32 mmol), K₂CO₃ (108 mg, 0.78 mmol) and Pd(PPh₃)₄ (6 mg, 0.0052 mmol) provided the coupling product as a white powder (92 mg, 100% yield). EIMS 353.1 [M+H]+.

### Example 80

### 9-(4-Chloro-2-methylphenyl)-2-methoxy-6,7-dimethylimidazo[1,5-a]pyrido[3,2-e]pyrazine

Following the general Suzuki coupling procedure, reaction of bromide 5B (80 mg, 0.26 mmol), 4-chloro-2-methylphenylboronic acid (53 mg, 0.32 mmol), K₂CO₃ (108 mg, 0.78 mmol) and Pd(PPh₃)₄ (6 mg, 0.0052 mmol) provided the coupling product as a white powder (85 mg, 92% yield). EIMS 353.1 [M+H]+.

### Example 81

### 9-(5-Fluoro-2-methylphenyl)-2-methoxy-6,7-dimethylmidazo[1,5-a]pyrido[3,2-e]pyrazine

Following the general Suzuki coupling procedure, reaction of bromide 5B (80 mg, 0.26 mmol), 5-fluoro-2-methylphenylboronic acid (48 mg, 0.32 mmol), K₂CO₃ (108 mg, 0.78 mmol) and Pd(PPh₃)₄ (6 mg, 0.0052 mmol) provided the coupling product as a white powder (83 mg, 95% yield). EIMS 337.1 [M+H]+.

### Example 82

### 9-(4-Fluoro-2-methylphenyl)-2-methoxy-6,7-dimethylmidazo[1,5-a]pyrido[3,2-e]pyrazine

Following the general Suzuki coupling procedure, reaction of bromide 5B (80 mg, 0.26 mmol), 4-fluoro-2-methylphenylboronic acid (48 mg, 0.32 mmol), K₂CO₃ (108 mg, 0.78 mmol) and Pd(PPh₃)₄ (6 mg, 0.0052 mmol) provided the coupling product as a white powder (67 mg, 77% yield). EIMS 337.1 [M+H]+.

### Example 83

### 9-(5-Fluoro-2-methoxyphenyl)-2-methoxy-6,7-dimethylimidazo[1,5-a]pyrido[3,2-e]pyrazine

Following the general Suzuki coupling procedure, reaction of bromide 5B (80 mg, 0.26 mmol), 5-fluoro-2-methoxyphenylboronic acid (53 mg, 0.32 mmol), K₂CO₃ (108 mg, 0.78 mmol) and Pd(PPh₃)₄ (6 mg, 0.0052 mmol) provided the coupling product as a white powder (89 mg, 98% yield). EIMS 353.1 [M+H]+.

### Example 84

### 9-(5-Chloro-2-methoxyphenyl)-2-methoxy-6,7-dimethylimidazo[1,5-a]pyrido[3,2-e]pyrazine

Following the general Suzuki coupling procedure, reaction of bromide 5B (80 mg, 0.26 mmol), 5-chloro-2-methoxyphenylboronic acid (58 mg, 0.32 mmol), K₂CO₃ (108 mg, 0.78 mmol) and Pd(PPh₃)₄ (6 mg, 0.0052 mmol) provided the coupling product as a white powder (96 mg, 100% yield). EIMS 369 [M+H]+.

### Example 85

### 4-(2-Methoxy-6,7-dimethylimidazo[1,5-a]pyrido[3,2-e]pyrazin-9-yl)benzamide

Following the general Suzuki coupling procedure, reaction of bromide 5B (80 mg, 0.26 mmol), 4-aminocarbonylphenylboronic acid (52 mg, 0.32 mmol), K₂CO₃ (108 mg, 0.78 mmol) and Pd(PPh₃)₄ (6 mg, 0.0052 mmol) provided the coupling product as a white powder (76 mg, 84% yield). EIMS 348.1 [M+H]+.

### Example 86

### 9-(4-Fluoro-2-methoxyphenyl)-2-methoxy-6,7-dimethylmidazo[1,5-a]pyrido[3,2-e]pyrazine

Following the general Suzuki coupling procedure, reaction of bromide 5B (80 mg, 0.26 mmol), 4-fluoro-2-methoxyphenylboronic acid (53 mg, 0.32 mmol), K₂CO₃ (108 mg, 0.78 mmol) and Pd(PPh₃)₄ (6 mg, 0.0052 mmol) provided the coupling product as a white powder (62 mg, 67% yield). EIMS 353.1 [M+H]+.

### Example 87

### 9-(3-Chloro-2-methylphenyl)-2-methoxy-6,7-dimethylimidazo[1,5-a]pyrido[3,2-e]pyrazine

Following the general Suzuki coupling procedure, reaction of bromide 5B (80 mg, 0.26 mmol), 3-chloro-2-methylphenylboronic acid (53 mg, 0.32 mmol), K₂CO₃ (108 mg, 0.78 mmol) and Pd(PPh₃)₄ (6 mg, 0.0052 mmol) provided the coupling product as a white powder (73 mg, 79% yield). EIMS 353.0 [M+H]+.

### Example 88

### 9-(3-Fluoro-2-methylphenyl)-2-methoxy-6,7-dimethylmidazo[1,5-a]pyrido[3,2-e]pyrazine

Following the general Suzuki coupling procedure, reaction of bromide 5B (80 mg, 0.26 mmol), 3-fluoro-2-methylphenylboronic acid (48 mg, 0.32 mmol), K₂CO₃ (108 mg, 0.78 mmol) and Pd(PPh₃)₄ (6 mg, 0.0052 mmol) provided the coupling product as a white powder (65 mg, 75% yield). EIMS 337.1 [M+H]+.

### Example 89

### 9-(2,3-dichlorophenyl)-2-methoxy-6,7-dimethylimidazo[1,5-a]pyrido[3,2-e]pyrazine

Following the general Suzuki coupling procedure, reaction of bromide 5B (80 mg, 0.26 mmol), 2,3-dichlorophenylboronic acid (60 mg, 0.32 mmol), K₂CO₃ (108 mg, 0.78 mmol) and Pd(PPh₃)₄ (6 mg, 0.0052 mmol) provided the coupling product as a white powder (82 mg, 85% yield). EIMS 373.0 [M+H]+.

### Example 90

### 9-(4-Chloro-2-methoxyphenyl)-2-methoxy-6,7-dimethylimidazo[1,5-a]pyrido[3,2-e]pyrazine

Following the general Suzuki coupling procedure, reaction of bromide 5B (80 mg, 0.26 mmol), 4-chloro-2-methoxyphenylboronic acid (58 mg, 0.32 mmol), K₂CO₃ (108 mg, 0.78 mmol) and Pd(PPh₃)₄ (6 mg, 0.0052 mmol) provided the coupling product as a white powder (78 mg, 81% yield). EIMS 369.2 [M+H]+.

### Example 91

### 9-[4-Chloro-2-(trifluoromethyl)phenyl]-2-methoxy-6,7-dimethylimidazo[1,5-a]pyrido[3,2-e]pyrazine

Following the general Suzuki coupling procedure, reaction of bromide 5B (80 mg, 0.26 mmol), 4-chloro-2-trifluoromethylphenylboronic acid (70 mg, 0.32 mmol), K₂CO₃ (108 mg, 0.78 mmol) and Pd(PPh₃)₄ (6 mg, 0.0052 mmol) provided the coupling product as a white powder (55 mg, 52% yield). EIMS 407.2 [M+H]+.

### Example 92

### 9-(5-Chloro-2-thienyl)-2-methoxy-6,7-dimethylimidazo[1,5-a]pyrido[3,2-e]pyrazine

Following the general Suzuki coupling procedure, reaction of bromide 5B (80 mg, 0.26 mmol), 5-chloro-2-thienylboronic acid (126 mg, 0.78 mmol), K₂CO₃ (108 mg, 0.78 mmol) and Pd(PPh₃)₄ (6 mg, 0.0052 mmol) provided the coupling product as a yellow powder (75 mg, 84% yield). EIMS 345.2 [M+H]+.

### Example 93

### 3-(2-Methoxy-6,7-dimethylimidazo[1,5-a]pyrido[3,2-e]pyrazin-9-yl)benzamide

Following the general Suzuki coupling procedure, reaction of bromide 5B (80 mg, 0.26 mmol), 3-aminocarbonylphenylboronic acid (52 mg, 0.32 mmol), K₂CO₃ (108 mg, 0.78 mmol) and Pd(PPh₃)₄ (6 mg, 0.0052 mmol) provided the coupling product as a white powder (80 mg, 89% yield). EIMS 348.1 [M+H]+.

### Example 94

### 2-Methoxy-9-[(3-methoxyphenyl)ethynyl]-6,7-dimethylimidazo[1,5-a]pyrido[3,2-e]pyrazine

Following the procedure of preparing Example 75, reaction of bromide 5B (80 mg, 0.26 mmol), DMF (3 mL), Et₃N (0.11 mL, 0.78 mmol), 3-methoxyphenylacetylene (42.2 mg, 0.32 mmol), Pd(PPh₃)₂Cl₂ (3.6 mg, 0.0052 mmol) and CuI (2 mg, 0.0104 mmol) provided the coupling product as a light yellow powder (78 mg, 84% yield). EIMS 359.1 [M+H]+.

### Example 95

### 9-(Cyclohexylethynyl)-2-methoxy-6,7-dimethylimidazo[1,5-a]pyrido[3,2-e]pyrazine

Following the procedure of preparing Example 75, reaction of bromide 5B (80 mg, 0.26 mmol), DMF (3 mL), Et₃N (0.11 mL, 0.78 mmol), cyclohexylacetylene (140 mg, 1.3 mmol), Pd(PPh₃)₂Cl₂ (3.6 mg, 0.0052 mmol) and CuI (2 mg, 0.0104 mmol) provided the coupling product as a light yellow powder (28 mg, 32% yield). EIMS 335.1 [M+H]+.

### Example 96

### 9-[(2-Chlorophenyl)ethynyl]-2-methoxy-6,7-dimethylimidazo[1,5-a]pyrido[3,2-e]pyrazine

Following the procedure of preparing Example 75, reaction of bromide 5B (80 mg, 0.26 mmol), DMF (3 mL), Et₃N (0.11 mL, 0.78 mmol), 2-chlorophenylacetylene (107 mg, 0.78 mmol), Pd(PPh₃)₂Cl₂ (3.6 mg, 0.0052 mmol) and CuI (2 mg, 0.0104 mmol) provided the coupling product as a light yellow powder (82 mg, 87% yield). EIMS 363.1 [M+H]+.

### Example 97

### 9-(Cyclopropylethynyl)-2-methoxy-6,7-dimethylmidazo[1,5-a]pyrido[3,2-e]pyrazine

Following the procedure of preparing Example 75, reaction of bromide 5B (80 mg, 0.26 mmol), DMF (3 mL), Et₃N (0.11 mL, 0.78 mmol), cyclopropylacetylene (172 mg, 2.6 mmol), Pd(PPh₃)₂Cl₂ (3.6 mg, 0.0052 mmol) and CuI (2 mg, 0.0104 mmol) provided the coupling product as a light yellow powder (34 mg, 45% yield). EIMS 293.1 [M+H]+.

### Example 98

### 2-Methoxy-9-[(2-methoxyphenyl)ethynyl]-6,7-dimethylimidazo[1,5-a]pyrido[3,2-e]pyrazine

Following the procedure of preparing Example 75, reaction of bromide 5B (80 mg, 0.26 mmol), DMF (3 mL), Et₃N (0.11 mL, 0.78 mmol), 2-methoxyphenylacetylene (42.2 mg, 0.32 mmol), Pd(PPh₃)₂Cl₂ (3.6 mg, 0.0052 mmol) and CuI (2 mg, 0.0104 mmol) provided the coupling product as a light yellow powder (25 mg, 27% yield). EIMS 359.1 [M+H]+.

### Example 99

### 2-Methoxy-6,7-dimethyl-9-[(2-methylphenyl)ethynyl]imidazo[1,5-a]pyrido[3,2-e]pyrazine

Following the procedure of preparing Example 75, reaction of bromide 5B (80 mg, 0.26 mmol), DMF (3 mL), Et₃N (0.11 mL, 0.78 mmol), 2-methylphenylacetylene (37 mg, 0.32 mmol), Pd(PPh₃)₂Cl₂ (3.6 mg, 0.0052 mmol) and CuI (2 mg, 0.0104 mmol) provided the coupling product as a light yellow powder (78 mg, 88% yield). EIMS 343.1 [M+H]+.

### Example 100

### 3-(2-Methoxy-6,7-dimethylimidazo[1,5-a]pyrido[3,2-e]pyrazin-9-yl)-N-methylbenzamide

Following the general Suzuki coupling procedure, reaction of bromide 5B (80 mg, 0.26 mmol), 3-(methylcarbamoyl)phenylboronic acid (56 mg, 0.32 mmol), K₂CO₃ (108 mg, 0.78 mmol) and Pd(PPh₃)₄ (6 mg, 0.0052 mmol) provided the coupling product as a light yellow powder (40 mg, 43% yield). EIMS 362.1 [M+H]+.

### Example 101

### N-Ethyl-3-(2-methoxy-6,7-dimethylimidazo[1,5-a]pyrido[3,2-e]pyrazin-9-yl)benzamide

Following the general Suzuki coupling procedure, reaction of bromide 5B (80 mg, 0.26 mmol), 3-(ethylcarbamoyl)phenylboronic acid (60 mg, 0.32 mmol), K₂CO₃ (108 mg, 0.78 mmol) and Pd(PPh₃)₄ (6 mg, 0.0052 mmol) provided the coupling product as a light yellow powder (52 mg, 53% yield). EIMS 376.1 [M+H]+.

### Example 102

### N-Methoxy-3-(2-methoxy-6,7-dimethylimidazo[1,5-a]pyrido[3,2-e]pyrazin-9-yl)benzamide

Following the general Suzuki coupling procedure, reaction of bromide 5B (80 mg, 0.26 mmol), 3-(methoxycarbamoyl)phenylboronic acid (62 mg, 0.32 mmol), K₂CO₃ (108 mg, 0.78 mmol) and Pd(PPh₃)₄ (6 mg, 0.0052 mmol) provided the coupling product as an off-white powder (65 mg, 66% yield). EIMS 378.0 [M+H]+.

### Example 103

### N-Isopropyl-3-(2-methoxy-6,7-dimethylimidazo[1,5-a]pyrido[3,2-e]pyrazin-9-yl)benzamide

Following the general Suzuki coupling procedure, reaction of bromide 5B (80 mg, 0.26 mmol), 3-(isopropylcarbamoyl)phenylboronic acid (65 mg, 0.32 mmol), K₂CO₃ (108 mg, 0.78 mmol) and Pd(PPh₃)₄ (6 mg, 0.0052 mmol) provided the coupling product as an off-white powder (66 mg, 65% yield). EIMS 390.1 [M+H]+.

### Example 104

### 3-(2-Methoxy-6,7-dimethylimidazo[1,5-a]pyrido[3,2-e]pyrazin-9-yl)-N,N-dimethylbenzamide

Following the general Suzuki coupling procedure, reaction of bromide 5B (80 mg, 0.26 mmol), 3-(dimethylcarbamoyl)phenylboronic acid (60 mg, 0.32 mmol), K₂CO₃ (108 mg, 0.78 mmol) and Pd(PPh₃)₄ (6 mg, 0.0052 mmol) provided the coupling product as an off-white powder (98 mg, 100% yield). EIMS 376.1 [M+H]+.

### Example 105

### 2-Methoxy-6,7-dimethyl-9-[3-(piperidin-1-ylcarbonyl)phenyl]imidazo[1,5-a]pyrido[3,2-e]pyrazine

Following the general Suzuki coupling procedure, reaction of bromide 5B (80 mg, 0.26 mmol), 3-(piperidine-1-carbonyl)phenylboronic acid (72 mg, 0.32 mmol), K₂CO₃ (108 mg, 0.78 mmol) and Pd(PPh₃)₄ (6 mg, 0.0052 mmol) provided the coupling product as an off-white powder (108 mg, 100% yield). EIMS 416.1 [M+H]+.

### Example 106

### 4-Fluoro-3-(2-methoxy-6,7-dimethylimidazo[1,5-a]pyrido[3,2-e]pyrazin-9-yl)benzamide

Following the general Suzuki coupling procedure, reaction of bromide 5B (80 mg, 0.26 mmol), 5-carbamoyl-2-fluorophenylboronic acid (58 mg, 0.32 mmol), K₂CO₃ (108 mg, 0.78 mmol) and Pd(PPh₃)₄ (6 mg, 0.0052 mmol) provided the coupling product as an off-white powder (89 mg, 93% yield). EIMS 366.1 [M+H]+.

### Example 107

### 4-Fluoro-3-(2-methoxy-6,7-dimethylimidazo[1,5-a]pyrido[3,2-e]pyrazin-9-yl)-N-methylbenzamide

Following the general Suzuki coupling procedure, reaction of bromide 5B (80 mg, 0.26 mmol), 2-fluoro-5-(methylcarbamoyl)phenylboronic acid (62 mg, 0.32 mmol), K₂CO₃ (108 mg, 0.78 mmol) and Pd(PPh₃)₄ (6 mg, 0.0052 mmol) provided the coupling product as an off-white powder (98 mg, 99% yield). EIMS 380.0 [M+H]+.

Examples 108-128 were prepared according to the processes described in this application or U.S. application Ser. Nos. 11/753,207 and 11/753,260.

TABLE 1
Examples 108-128

| Example | Chemical Structure |
|---|---|
| 108 | 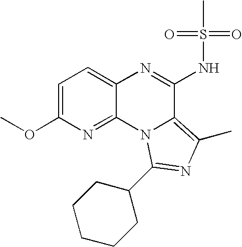 |
| 109 | 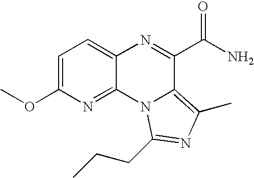 |
| 110 | 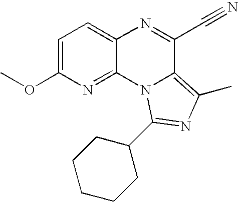 |
| 111 | 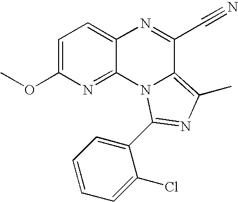 |
| 112 | 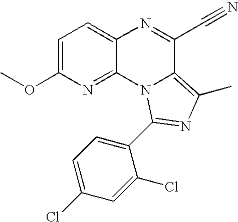 |
| 113 | 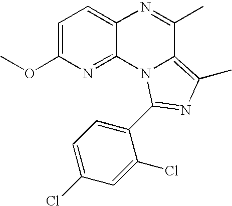 |
| 114 | 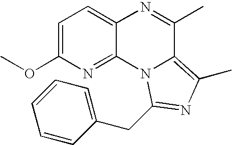 |
| 115 | 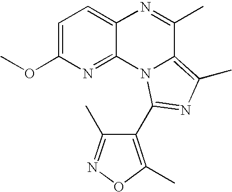 |
| 116 | 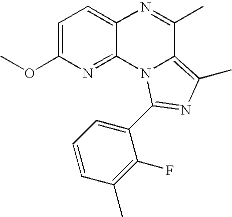 |
| 117 | 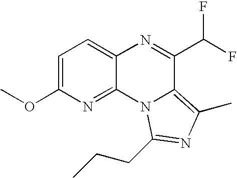 |
| 118 | 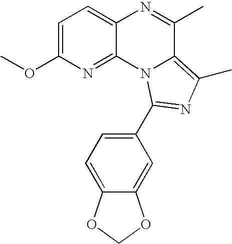 |
| 119 | 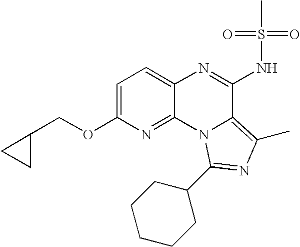 |
| 120 | 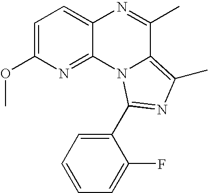 |
| 121 | 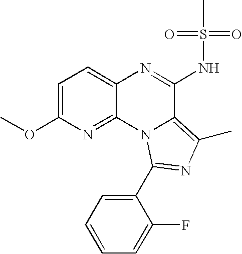 |
| 122 | 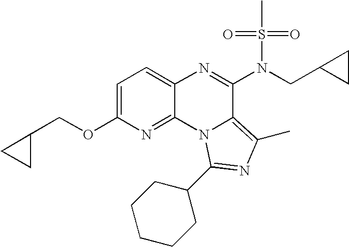 |
| 123 | 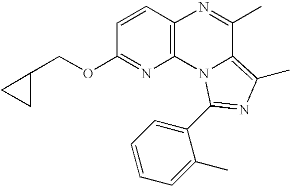 |
| 124 | 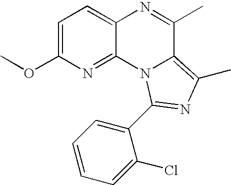 |
| 125 | 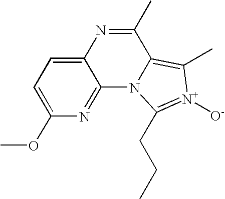 |
| 126 | 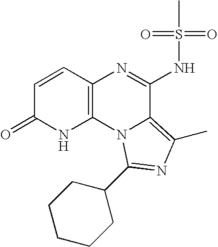 |
| 127 | 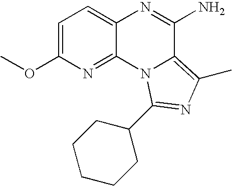 |
| 128 | 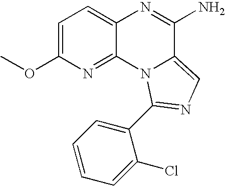 |

Examples 129-132 were prepared according to the method described in Example 47.

TABLE 2
Examples 129-132

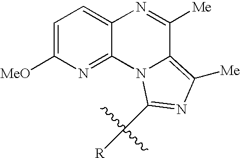

The symbol “ [symbol] ” shows the point where substituent R is
attached to the tricyclic ring system

| Example | R | Chemical Name |
|---|---|---|
| 129 | 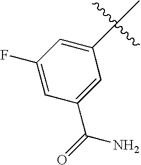 | 3-fluoro-5-(2-methoxy-6,7- dimethylimidazo[1,5- a]pyrido[3,2-e]pyrazin- 9-yl)benzamide |
| 130 | 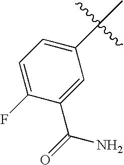 | 2-fluoro-5-(2-methoxy-6,7- dimethylimidazo[1,5- a]pyrido[3,2-e]pyrazin- 9-yl)benzamide |
| 131 | 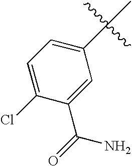 | 2-chloro-5-(2-methoxy-6,7- dimethylimidazo[1,5- a]pyrido[3,2-e]pyrazin- 9-yl)benzamide |
| 132 | 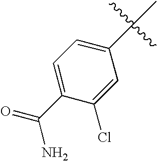 | 2-chloro-4-(2-methoxy-6,7- dimethylimidazo[1,5- a]pyrido[3,2-e]pyrazin- 9-yl)benzamide |

### Example 133

### (2-Methoxy-6,7-dimethylimidazo[1,5-a]pyrido[3,2-e]pyrazin-9-yl)acetonitrile

Scheme 11 shows a synthetic method that was used in the preparation of compounds of Example 133.

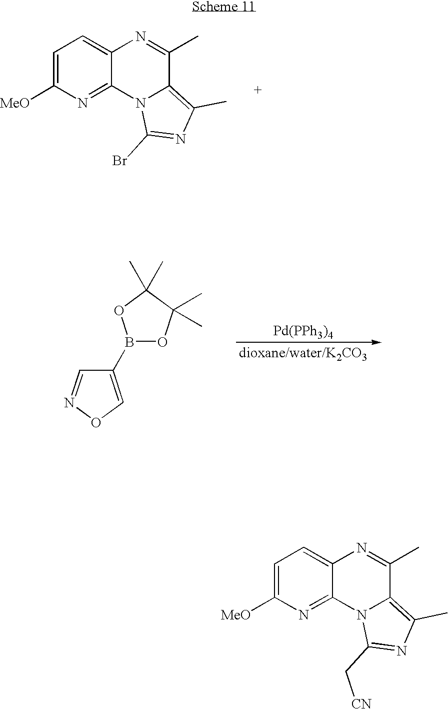

Following the General Experimental for Suzuki Coupling as shown in Scheme 11, the above named compound was obtained as an off-white powder. [M+H]⁺ 268.1 (ESI).

Examples 134-151 were prepared according to Scheme 12.

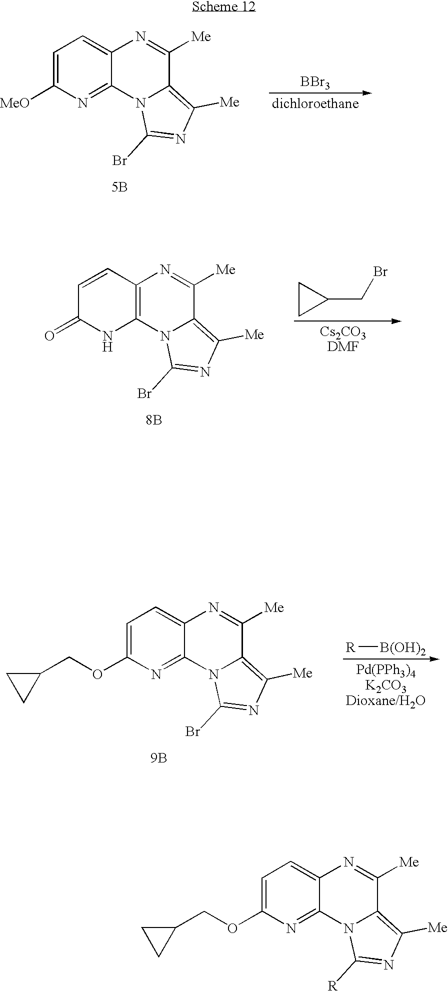

### 9-Bromo-6,7-dimethylimidazo[1,5-a]pyrido[3,2-e]pyrazin-2(1H)-one (8B)

To a mixture of substrate 5B (306 mg, 1 mmol) in 10 mL dichloroethane was added BBr₃ (0.8 mL, 8 mmol) dropwise at 0° C. The resulting mixture was stirred at 80° C. overnight and then cooled to room temperature, poured into a solution of 2 g K₂CO₃ in 20 mL ice water. The crude product precipitated and was filtered, which was purified by column using 5% methanol in dichloromethane as eluent to provide 160 mg (55% yield) of product 8B as a yellow powder. [M+H]⁺ 292.9 (ESI).

### 9-Bromo-2-(cyclopropylmethoxy)-6,7-dimethylimidazo[1,5-a]pyrido[3,2-e]pyrazine (9B)

To a mixture of substrate 8B (160 mg, 0.54 mmol) and Cs₂CO₃ (266 mg, 0.82 mmol) in 5 mL DMF was added cyclopropylmethyl bromide (0.08 mL, 0.82 mmol). The resulting mixture was warmed to 100° C. overnight, cooled to room temperature and diluted with water. Standard workup followed by column purification provided 156 mg (84% yield) of product 9B as a yellow solid. [M+H]⁺ 347.0 (ESI).

TABLE 3
Examples 134-151

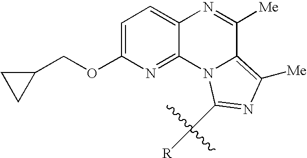

The symbol “ [symbol] ” shows the point where substituent R is attached
to the tricyclic ring system

| Exam- | | |
|---|---|---|
| ple | R | Chemical Name |
| 134 | 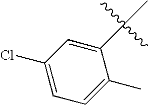 | 9-(5-chloro-2-methylphenyl)-2- (cyclopropylmethoxy)-6,7- dimethylimidazo[1,5- a]pyrido[3,2-e]pyrazine |
| 135 | 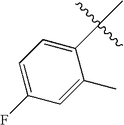 | 2-(cyclopropylmethoxy)-9-(4- fluoro-2-methylphenyl)-6,7- dimethylimidazo[1,5- a]pyrido[3,2-e]pyrazine |
| 136 | 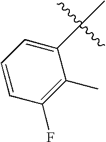 | 2-(cyclopropylmethoxy)-9-(3- fluoro-2-methylphenyl)-6,7- dimethylimidazo[1,5- a]pyrido[3,2-e]pyrazine |
| 137 | 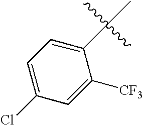 | 9-[4-chloro-2- (trifluoromethyl)phenyl]-2- (cyclopropylmethoxy)-6,7- dimethylimidazo[1,5- a]pyrido[3,2-e]pyrazine |
| 138 | 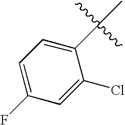 | 9-(2-chloro-4-fluorophenyl)-2- (cyclopropylmethoxy)-6,7- dimethylimidazo[1,5- a]pyrido[3,2-e]pyrazine |
| 139 | 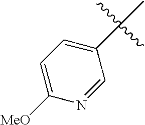 | 2-(cyclopropylmethoxy)-9-(6- methoxypyridin-3-yl)-6,7- dimethylimidazo[1,5- a]pyrido[3,2-e]pyrazine |
| 140 | 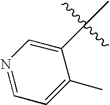 | 2-(cyclopropylmethoxy)-6,7- dimethyl-9-(4-methylpyridin-3- yl)imidazo[1,5-a]pyrido[3,2- e]pyrazine |
| 141 | 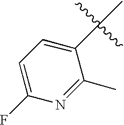 | 2-(cyclopropylmethoxy)-9-(6- fluoro-2-methylpyridin-3-yl)-6,7- dimethylimidazo[1,5- a]pyrido[3,2-e]pyrazine |
| 142 | 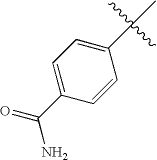 | 4-[2-(cyclopropylmethoxy)-6,7- dimethylimidazo[1,5- a]pyrido[3,2-e]pyrazin-9- yl]benzamide |
| 143 | 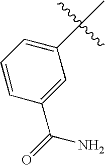 | 3-[2-(cyclopropylmethoxy)-6,7- dimethylimidazo[1,5- a]pyrido[3,2-e]pyrazin-9- yl]benzamide |
| 144 | 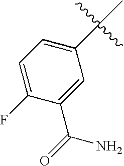 | 5-(2-(cyclopropylmethoxy)-6,7- dimethylimidazo[1,5- a]pyrido[3,2-e]pyrazin-9-yl)-2- fluorobenzamide |
| 145 | 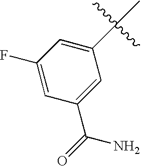 | 3-[2-(cyclopropylmethoxy)-6,7- dimethylimidazo[1,5- a]pyrido[3,2-e]pyrazin-9-yl]-5- fluorobenzamide |
| 146 | 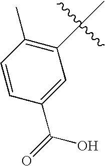 | 3-[2-(cyclopropylmethoxy)-6,7- dimethylimidazo[1,5- a]pyrido[3,2-e]pyrazin-9-yl]-4- methylbenzoic acid |
| 147 | 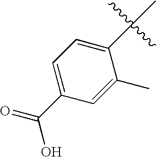 | 4-[2-(cyclopropylmethoxy)-6,7- dimethylimidazo[1,5- a]pyrido[3,2-e]pyrazin-9-yl]-3- methylbenzoic acid |
| 148 | 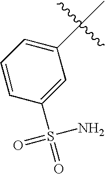 | 3-[2-(cyclopropylmethoxy)-6,7- dimethylimidazo[1,5- a]pyrido[3,2-e]pyrazin-9- yl]benzenesulfonamide |
| 149 | 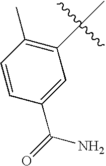 | 3-[2-(cyclopropylmethoxy)-6,7- dimethylimidazo[1,5- a]pyrido[3,2-e]pyrazin-9-yl]-4- methylbenzamide |
| 150 | 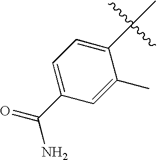 | 4-[2-(cyclopropylmethoxy)-6,7- dimethylimidazo[1,5- a]pyrido[3,2-e]pyrazin-9-yl]-3- methylbenzamide |
| 151 | 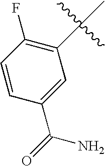 | 3-[2-(cyclopropylmethoxy)-6,7- dimethylimidazo[1,5- a]pyrido[3,2-e]pyrazin-9-yl]-4- fluorobenzamide |

Examples 123 and 152-158 were prepared according to Scheme 13.

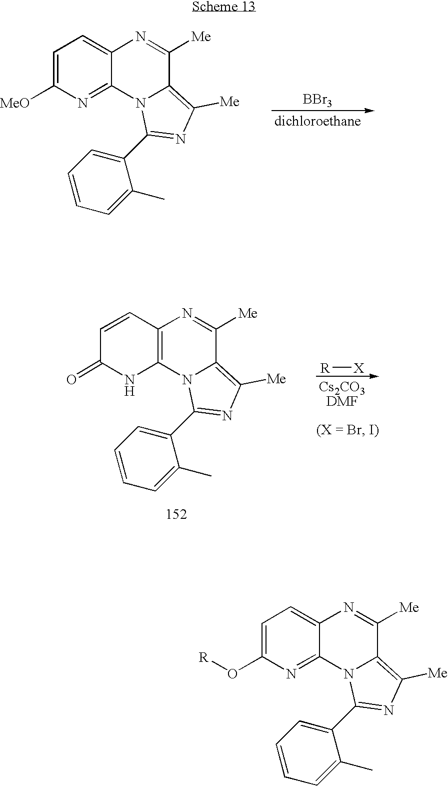

### Example 152

### 6,7-Dimethyl-9-o-tolylimidazo[1,5-a]pyrido[3,2-e]pyrazin-2(1H)-one

The procedure to prepare compound 8B in Scheme 12 was followed to prepare Example 152, which was isolated as a yellow powder (82% yield). [M+H]⁺ 305.1 (ESI).

TABLE 4
Examples 123 and 153-158

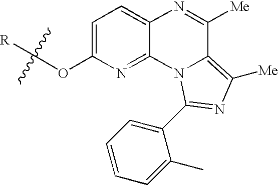

The symbol “ [symbol] ” shows the point where substituent R is
attached to the tricyclic ring system

| Exam- | | |
|---|---|---|
| ple | R | Chemical Name |
| 153 | 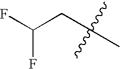 | 2-(2,2-difluoroethoxy)-6,7- dimethyl-9-(2- methylphenyl)imidazo[1,5- a]pyrido[3,2-e]pyrazine |
| 154 | 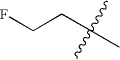 | 2-(2-fluoroethoxy)-6,7- dimethyl-9-(2- methylphenyl)imidazo[1,5- a]pyrido[3,2-e]pyrazine |
| 155 | 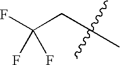 | 6,7-dimethyl-9-(2- methylphenyl)-2-(2,2,2- trifluoroethoxy)imidazo[1,5- e]pyrazine |
| 123 | 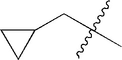 | 2-(cyclopropylmethoxy)-6,7- dimethyl-9-(2- methylphenyl)imidazo[1,5- a]pyrido[3,2-e]pyrazine |
| 156 | 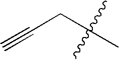 | 6,7-dimethyl-9-(2- methylphenyl)-2-(prop-2-yn- 1-yloxy)imidazo[1,5- a]pyrido[3,2-e]pyrazine |
| 157 | 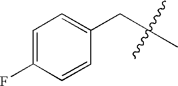 | 2-[(4-fluorobenzyl)oxy]-6,7- dimethyl-9-(2- methylphenyl)imidazo[1,5- a]pyrido[3,2-e]pyrazine |
| 158 | 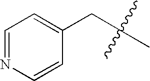 | 6,7-dimethyl-9-(2- methylphenyl)-2-(pyridin-4- ylmethoxy)imidazo[1,5- a]pyrido[3,2-e]pyrazine |

### Example 159

### 6,7-Dimethyl-9-(4-methylpyridin-3-yl)imidazo[1,5-a]pyrido[3,2-e]pyrazin-2(H)-one

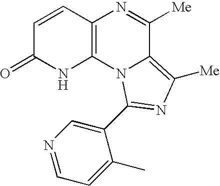

6,7-Dimethyl-9-(4-methylpyridin-3-yl)imidazo[1,5-a]pyrido[3,2-e]pyrazin-2(1H)-one was prepared according to Scheme 13. It was isolated as a yellow powder. ¹H NMR (400 MHz, DMSO) δ ppm 10.98 (s, br, 1H), 8.50 (d, 1H), 8.43 (s, 1H), 8.01 (d, 1H), 7.33 (d, 1H), 6.76 (d, 1H), 2.75 (s, 3H), 2.71 (s, 3H), 2.02 (s, 3H). [M+H]⁺ 306.1 (ESI).

### Example 160

### 2-Methoxy-6,7-dimethyl-9-(3-methylpyridin-4-yl)imidazo[1,5-a]pyrido[3,2-e]pyrazine

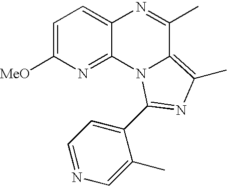

Compound was made according to Example 5. A white solid was recovered (0.06 g) 48% yield. MS (ES) m/z 320.1 [M+1]⁺

### Example 161

### 2-Methoxy-9-(3-methoxypyridin-4-yl)-6,7-dimethylimidazo[1,5-a]pyrido[3,2-e]pyrazine

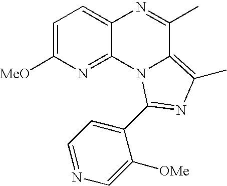

Compound was made according to Example 5. A white solid was recovered (0.06 g) 46% yield. MS (ES) m/z 336.1 [M+1]⁺

### Example 162

### 2-Methoxy-6,7-dimethyl-9-(6-methylpyridin-3-yl)imidazo[1,5-a]pyrido[3,2-e]pyrazine

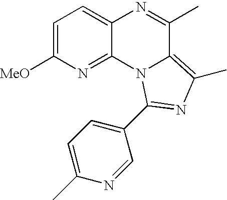

Compound was made according to Example 5. A white solid was recovered (0.1 g) 80% yield. MS (ES) m/z 320.2 [M+1]⁺

Scheme 14 shows a synthetic method that was used in the preparation of Intermediate 1 used in the preparation of Example 163.

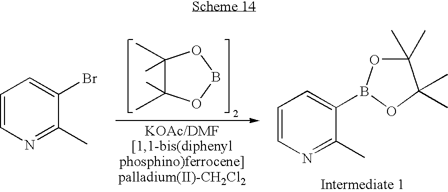

### Intermediate 1: 2-Methyl-3-(4,4,5,5-tetramethyl-[1,3,2]dioxaborolan-2-yl)-pyridine

3-Bromo-2-pinnacol (1 g, 5.8 mmol) was dissolved in DMF (20 mL). To this was added potassium acetate (2 g, 20.3 mmol) followed by 4,4,5,5,4′,4′,5′,5′-octamethyl-[2,2′]bi[[1,3,2]dioxaborolanyl] (1.9 g, 7.5 mmol) and [1,1-bis(diphenylphosphino)ferrocene]palladium(II) bis methylene chloride (0.47 g, 10% mol).

The reaction was heated to 80° C. for 16 hrs then poured into water and extracted with ethyl acetate. The organic layer was separated and brined then dried over magnesium sulfate. The solvent was removed under reduced pressure and the crude purified by flash chromatography on silica gel in ethyl acetate. A greenish/black oil 0.2 g was recovered.

### Example 163

### 2-Methoxy-6,7-dimethyl-9-(2-methylpyridin-3-yl)imidazo[1,5-a]pyrido[3,2-e]pyrazine

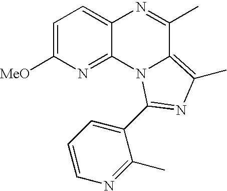

Compound was made according to Example 5 using intermediate 1 (2-methyl-3-(4,4,5,5-tetramethyl-[1,3,2]dioxaborolan-2-yl)-pyridine).

A white solid was recovered (0.15 g) 73% yield. MS (ES) m/z 320.1 [M+1]⁺

¹H NMR (400 MHz, DMSO) δ ppm 8.50 (m, 1H), 8.10 (d, 1H), 7.75 (dxd, 1H), 7.35 (m, 1H), 6.15 (d, 1H), 3.05 (s, 1H), 2.75 (s, 3H), 2.70 (s, 3H), 2.15 (s, 3H).

### Example 164

### 9-Bromo-2-methoxy-7-(trifluoromethyl)imidazo[1,5-a]pyrido[3,2-e]pyrazine-6-carbonitrile

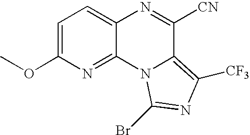

A mixture of imidazo[1,5-a]pyrido[3,2-e]pyrazine, 9-bromo-6-chloro-2-methoxy-7-(trifluoromethyl) (5.0 g, 13.1 mmol), DMSO (100 mL) and tetraethylammonium cyanide (2.0 g, 13.1 mmol) was stirred at 75° C. for 10 hours. The mixture was poured into water and extracted with CH₂Cl₂. The organic extracts were dried over MgSO₄. Evaporation and purification by ISCO (eluting solvent CH₂Cl₂/EtOAc 3/1) afforded 9-bromo-2-methoxy-7-(trifluoromethyl)imidazo[1,5-a]pyrido[3,2-e]pyrazine-6-carbonitrile as a yellow solid (3.85 g, 79% yield); MS m/s 317(M+)

Examples 111, 112, and 165-172 were prepared according to Example 40.

Intermediate F (9-bromo-2-methoxy-7-methylimidazo[1,5-a]pyrido[3,2-e]pyrazine-6-carbonitrile) of Scheme 7 was coupled with the corresponding boronic acids or boronic acid pinacol esters under palladium catalyzed conditions.

TABLE 5
Examples 111, 112, and 165-172

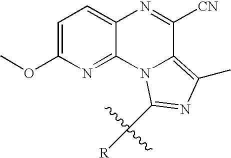

The symbol “ [symbol] ” shows the point where substituent R is
attached to the tricyclic ring system

| Exam- | | |
|---|---|---|
| ple | R | Chemical Name |
| 165 | 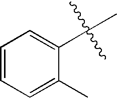 | 2-methoxy-7-methyl-9-(2- methylphenyl)imidazo[1,5- a]pyrido[3,2-e]pyrazine-6- carbonitrile |
| 166 | 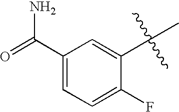 | 3-(6-cyano-2-methoxy-7- methylimidazo[1,5- a]pyrido[3,2-e]pyrazin-9-yl)- 4-fluorobenzamide |
| 167 | 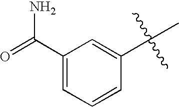 | 3-(6-cyano-2-methoxy-7- methylimidazo[1,5- a]pyrido[3,2-e]pyrazin-9- yl)benzamide |
| 168 | 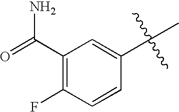 | 5-(6-cyano-2-methoxy-7- methylimidazo[1,5- a]pyrido[3,2-e]pyrazin-9-yl)- 2-fluorobenzamide |
| 169 | 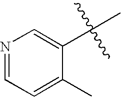 | 2-methoxy-7-methyl-9-(4- methylpyridin-3- yl)imidazo[1,5-a]pyrido[3,2- e]pyrazine-6-carbonitrile |
| 170 | 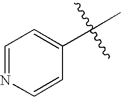 | 2-methoxy-7-methyl-9- pyridin-4-ylimidazo[1,5- a]pyrido[3,2-e]pyrazine-6- carbonitrile |
| 171 | 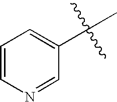 | 2-methoxy-7-methyl-9- pyridin-3-ylimidazo[1,5- a]pyrido[3,2-e]pyrazine-6- carbonitrile |
| 172 | 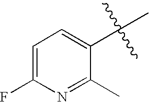 | 9-(6-fluoro-2-methylpyridin- 3-yl)-2-methoxy-7- methylimidazo[1,5- a]pyrido[3,2-e]pyrazine-6- carbonitrile |
| 111 | 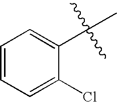 | 9-(2-chlorophenyl)-2- methoxy-7- methylimidazo[1,5- a]pyrido[3,2-e]pyrazine-6- carbonitrile |
| 112 | 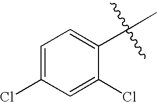 | 9-(2,4-dichlorophenyl)-2- methoxy-7- methylimidazo[1,5- a]pyrido[3,2-e]pyrazine-6- carbonitrile |

Examples 173-191 were prepared according to Example 47.

Intermediate E (9-bromo-2-methoxy-6,7-dimethylimidazo[1,5-a]pyrido[3,2-e]pyrazine) of Scheme 9 was coupled with the corresponding boronic acids or boronic acid pinacol esters under palladium catalyzed conditions.

TABLE 6
Examples 173-191

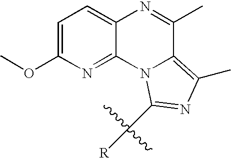

The symbol “ [symbol] ” shows the point where substituent R is
attached to the tricyclic ring system

| Exam- | | |
|---|---|---|
| ple | R | Chemical Name |
| 173 | 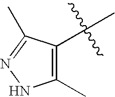 | 9-(3,5-dimethyl-1H-pyrazol-4- yl)-2-methoxy-6,7- dimethylimidazo[1,5- a]pyrido[3,2-e]pyrazine |
| 174 | 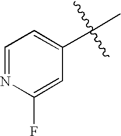 | 9-(2-fluoropyridin-4-yl)-2- methoxy-6,7- dimethylimidazo[1,5- a]pyrido[3,2-e]pyrazine |
| 175 | 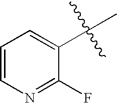 | 9-(2-fluoropyridin-3-yl)-2- methoxy-6,7- dimethylimidazo[1,5- a]pyrido[3,2-e]pyrazine |
| 176 | 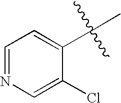 | 9-(3-chloropyridin-4-yl)-2- methoxy-6,7- dimethylimidazo[1,5- a]pyrido[3,2-e]pyrazine |
| 177 | 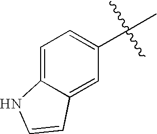 | 9-(1H-indol-5-yl)-2-methoxy- 6,7-dimethylimidazo[1,5- a]pyrido[3,2-e]pyrazine |
| 178 | 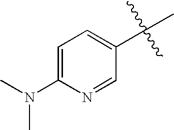 | 5-(2-methoxy-6,7- dimethylimidazo[1,5- a]pyrido[3,2-e]pyrazin-9-yl)- N,N-dimethylpyridin-2-amine |
| 179 | 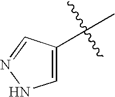 | 2-methoxy-6,7-dimethyl-9- (1H-pyrazol-4-yl)imidazo[1,5- a]pyrido[3,2-e]pyrazine |
| 180 | 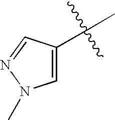 | 2-methoxy-6,7-dimethyl-9-(1- methyl-1H-pyrazol-4- yl)imidazo[1,5-a]pyrido[3,2- e]pyrazine |
| 181 | 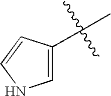 | 2-methoxy-6,7-dimethyl-9- (1H-pyrrol-3-yl)imidazo[1,5- a]pyrido[3,2-e]pyrazine |
| 182 | 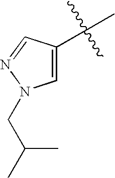 | 2-methoxy-6,7-dimethyl-9-[1- (2-methylpropyl)-1H-pyrazol- 4-yl]imidazo[1,5- yl}imidazo[1,5-a]pyrido[3,2- e]pyrazine |
| 183 | 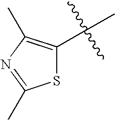 | 9-(2,4-dimethyl-1,3-thiazol-5- yl)-2-methoxy-6,7- dimethylimidazo[1,5- a]pyrido[3,2-e]pyrazine |
| 184 | 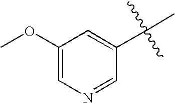 | 2-methoxy-9-(5- methoxypyridin-3-yl)-6,7- dimethylimidazo[1,5- a]pyrido[3,2-e]pyrazine |
| 185 | 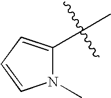 | 2-methoxy-6,7-dimethyl-9-(1- methyl-1H-pyrrol-2- yl)imidazo[1,5-a]pyrido[3,2- e]pyrazine |
| 186 | 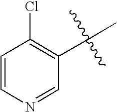 | 9-(4-chloropyridin-3-yl)-2- methoxy-6,7- dimethylimidazo[1,5- a]pyrido[3,2-e]pyrazine |
| 187 | 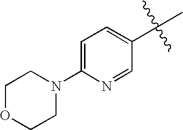 | 2-methoxy-6,7-dimethyl-9-(6- morpholin-4-ylpyridin-3- yl)imidazo[1,5-a]pyrido[3,2- e]pyrazine |
| 188 | 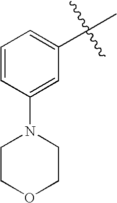 | 2-methoxy-6,7-dimethyl-9-(3- morpholin-4- ylphenyl)imidazo[1,5- a]pyrido[3,2-e]pyrazine |
| 189 | 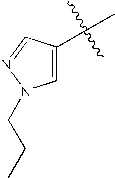 | 2-methoxy-6,7-dimethyl-9-(1- propyl-1H-pyrazol-4- yl)imidazo[1,5-a]pyrido[3,2- e]pyrazine |
| 190 | 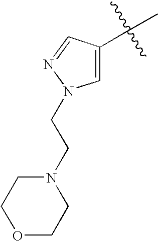 | 2-methoxy-6,7-dimethyl-9-[1- (2-morpholin-4-ylethyl)-1H- pyrazol-4-yl]imidazo[1,5- a]pyrido[3,2-e]pyrazine |

### Example 191

### 2-Methoxy-6,7-dimethyl-9-(1,3,5-trimethyl-1H-pyrazol-4-yl)imidazo[1,5-a]pyrido[3,2-e]pyrazine

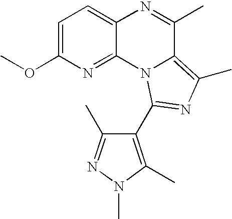

¹H NMR (400 MHz, DMSO) δ ppm 8.00 (d, 1H), 7.85 (d, 1H), 3.65 (s, 3H), 3.35 (s, 3H), 2.75 (s, 3H), 2.70 (s, 3H), 2.00 (s, 3H), 1.85 (s, 3H).

### Example A

### Inhibition of PDE10

### Method A

Phosphodiesterase isoenzyme 10 (PDE10) activity was determined in preparations of human recombinant PDE10A and PDE10 from pig striatum, respectively.

The DNA of PDE10A1 (AB 020593, 2340 bp) was synthesized and cloned into the vector pCR4.TOPO (Entelechon GmbH, Regensburg, Germany). The gene was than inserted into a baculovirus vector, ligated with the baculovirus DNA. The enzyme-protein was expressed in SF21-cells. The enzyme was isolated from these cells by harvesting the cells by a centrifugation at 200 g to collect the cells. The cells were resuspended in 50 mM Tris-HCl/5 mM MgCl₂ buffer (pH=7.4) and lysed by a sonication of the cells. The cytosolic PDE10A was obtained by a centrifugation at 48000 g for 1 h in the supernatant and stored at −70° C.

Striatum from male hybrid pigs (150 kg) were collected and frozen at −70° C.

At the day of preparation 0.5 g striatum was homogenised in 10 ml 50 mM Tris/Mg-buffer at 4° C. and centrifuged for one hour at 100000 g. The supernatant was removed and the pellet was resuspended in the same buffer, but containing 1% Triton and incubated for 45 min at 4° C. The membrane fraction was applied onto a 5 ml Hi Trap™ QHP column at the Akta-FPLC. After washing the column the bound PDE protein was eluted with an increasing sodium chloride gradient (O mM-500 mM sodium chloride) in 50 mM Tris/Mg-buffer at 4° C. in the presence of 1% Triton. The eluted and collected fractions were tested with 100 nM [3H]-cAMP for PDE10-activity in the presence and without a specific PDE-Inhibitor at a concentration, were a 100% inhibition is expected. The fractions with PDE10-activity were pooled and frozen in aliquots until use at −20° C.

PDE10 activity was determined in a one step procedure in microtiterplates. The reaction mixture of 100 μl contained 50 mM Tris-HCl/5 mM MgCl₂ buffer (pH=7.4) (Sigma, Deisenhofen, Germany; Merck, Darmstadt, Germany) 0.1 μM [3H]-cAMP (Amersham, Buckinghamshire, UK) and the enzyme. Nonspecific activity was tested without the enzyme. The reaction was initiated by addition of the substrate solution and was carried out at 37° C. for 30 minutes. Enzymatic activity was stopped by addition of 25 μl YSi-SPA-beads (Amersham-Pharmacia). One hour later the mixture was measured in a liquid scintillation counter for microtiterplates (Microbeta Trilux). To pipette the incubation mixture a robot Biomek (Fa. Beckman) is used. The determined Km-values for the substrate cAMP is 88 nM for pig striatum and 130 nM for human recombinant PDE10A respectively. The optimal amount of enzyme in the assay has been determined and optimised for each enzyme preparation before using the enzyme in compound testing. For determination of IC50 values the Hill-plot, 2-parameter-model, was used. Specific inhibitors of other PDE-Subtypes do not inhibit the PDE10 preparation significantly. Papaverine was used as the most common PDE10 inhibitor and inhibits the PDE10 with IC50 values of 89 nM and 103 nM for PDE10 from human recombinant PDE10A and PDE10 from striatum of pig respectively.

### Method B

The phosphodiesterase isoenzyme 10 (PDE10) activity was determined in preparations of rat, pig and guinea pig striatum respectively. Striatum from male Wistar rats (180-200 g), male hybrid pigs (150 kg) and male guinea pigs (CRL (HA), 500 g) respectively were collected and frozen at −70° C.

At the day of preparation 0.5 g striatum was homogenized in 10 ml 50 mM Tris/Mg-buffer at 4° C. and centrifuged for one hour at 100000 g. The supernatant is called the cytosolic fraction and was removed and stored on ice. The pellet was resuspended in the same buffer, but containing 1% Triton and incubated for 45 min at 4° C. Both fractions were independently applied onto a 5 ml Hi Trap™ QHP column at the Akta-FPLC. After washing the columns the bound PDE protein was eluted with an increasing sodium chloride gradient (0 mM-500 mM sodium chloride) in 50 mM Tris/Mg-buffer at 4° C. for the cytosolic fraction and in the presence of 1% Triton for the membrane fraction. The eluted and collected fractions were tested with 100 nM [³H]-cAMP for PDE10-activity in the presence and without a specific PDE-Inhibitor at a concentration, were a 100% inhibition is expected. The fractions with PDE10-activity were pooled and frozen in aliquots until use at −20° C.

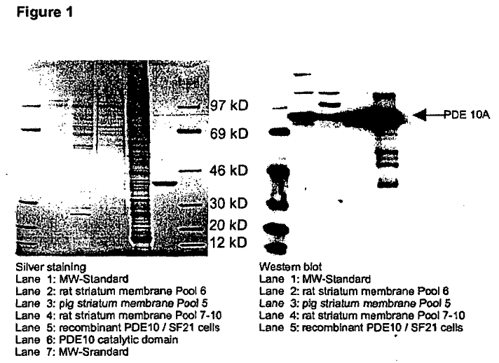

The eluted fractions from the FPLC were additionally characterized by Western blot (FIG. 1).

It was shown, that the PDE10A containing pooled fractions include a great number of other proteins. Nevertheless PDE10 was detected with specific antibodies by Western blot clearly.

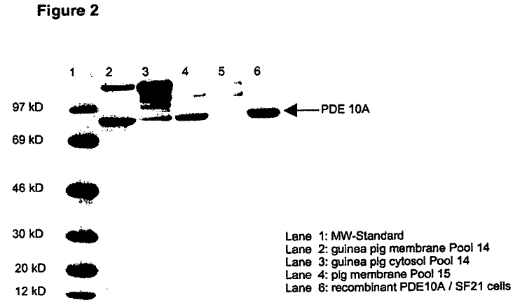

The protein was proven in the preparation of the striatum of the rat, the pig and the guinea pig. The main part of protein was found in the membrane fraction (FIG. 2).

In the prepared brain areas gene segments containing the catalytic domain of the PDE10 were amplified and the sequence determined.

Therefore the RNA from the frozen striatum of the different animals was isolated according to the instructions of the RNeasy kit (Qiagen; Hilden; Germany) and transcribed into cDNA using Oligo-Primer provided with the 1st strand cDNA synthese kit for RT-PCR (Roche; Mannheim; Germany). These cDNA was used as template for the PCR-reaction to amplify the catalytic domain of the PDE10. For the PCR reaction Taq-Polymerase (Promega; Mannheim; Germany) was used. Therefore it was possible to clone the amplificates directly by TA-cloning in the pCR2.1 vector (Invitrogen; Karlsruhe; Germany). The cloning vector was transformed into E. coli's (XL-2), replicated within the cells, prepared and the included gene sequence determined for the pig and the guinea pig.

The following primers were used for the PCR-reaction:

| | | |
|---|---|---|
| P1: | tgcatctacagggttaccatggagaa | (SEQ ID NO: 1) |
| P2: | tatccctgcaggccttcagcagaggctct | (SEQ ID NO: 2) |
| P3: | ttcacatggatatgcgacggtaccttct | (SEQ ID NO: 3) |
| P4: | ctgtgaagaagaactatcggcgggttcctta. | (SEQ ID NO: 4) |

For the pig the priming was successful with P1 and P2. The following sequence (SEQ ID NO: 5) was identified:

tgcatctacagggttaccatggagaagctgtcctaccacagcatttgtac
cgcggaagagtggcaaggcctcatgcgcttcaaccttcccgtccgtcttt
gcaaggagattgaattgttccacttcgacattggtccttttgaaaacatg
tggcctggaatctttgtctatatggttcatcgcttctgtgggacggcctg
ctttgagcttgaaaagctgtgtcgttttatcatgtctgtgaagaagaact
atcgtcgggttccttaccacaactggaagcacgcggtcacggtggcacac
tgcatgtacgccatcctccagaacagccacgggctcttcaccgacctcga
gcgcaaaggactgctaatcgcgtgtctgtgccacgacctggaccacaggg
gcttcagcaacagctacctgcagaaattcgaccaccccctggccgctctc
tactccacgcccaccatggagcagcaccacttctcccagaccgtgtccat
cctccagttggaagggcacaacatcttctccaccctgagctccagtgagt
acgagcaggtgcttgagatcatccgcaaagccatcattgccacagacctc
gctttgtactttggaaacaggaaacagttggaggagatgtaccagaccgg
atcgctaaaccttaataaccagtcacatagagaccgcgtcattggtttga
tgatgactgcctgtgatctctgttccgtgacaaaactgtggccagtaaca
aaactgacggcaaatgatatatatgcggaattctgggccgagggcgatga
ggtgaagaagctgggaatacagcctattcccatgatggacagagacaaga
aggacgaagtcccacaaggccagctcggattctacaacgcggtagctatc
ccctgctacaccaccctcacccagatcttcccgcccacagagcctcttct
gaaggcctgcagggata

For the guinea pig the priming was successful with P4 and P2 as well as for P2 and P3. The following sequence (SEQ ID NO: 6) was identified with P4 and P2:

ctgtgaagaagaactatcggcgggttccttaccacaactggaagcatgca
gtcacggtggcgcactgcatgtacgccatacttcaaaacaacaatggcct
cttcacagaccttgagcgcaaaggcctgctaattgcctgtctgtgccatg
acctggaccacaggggcttcagtaacagctacctgcagaaattcgaccac
cccctggctgcgttgtactccacctccaccatggagcaacaccacttctc
ccagacggtgttcatcctccagctggaaggacacaacatcttctccaccc
tgagctccagcgagtacgagcaggtgctggagatcatccgcaaagccatc
atcgccactgacctcgcactgtactttgggaacaggaagcagttggagga
gatgtaccagacagggtcgctgaacctcaataaccagtcccatcgagacc
gcgtcatcggcttgatgatgactgcctgcgatctttgctctgtgacgaaa
ctatggccagttacaaaattgacagcaaatgatatatatgcagagttctg
ggctgagggggatgagatgaagaagttggggatacagcccatccctatga
tggacagagacaagaaggatgaagtccctcaaggacagcttggattctac
aatgctgtggccatcccctgctataccaccctgacgcagatcctcccacc
cacagagcctctgctgaaggcctgcagggata

The following sequence (SEQ ID NO: 7) was identified with P2 and P3:

tagagcctctgctgaaggcctgcagggataacctcaatcagtgggagaag
gtaattcgaggggaagagacagcaatgtggatttcaggcccagcaactag
caaaagcacatcagggaagccgaccaggaaggtcgatgactgatcctgag
gtgatgtctgcctagcaactgactcaacctgcttctgtgacttcgttctt
tttatttttatttttttaacggggtgaaaacctctctcagaaggtaccgt
cgcatatccatgtgaa

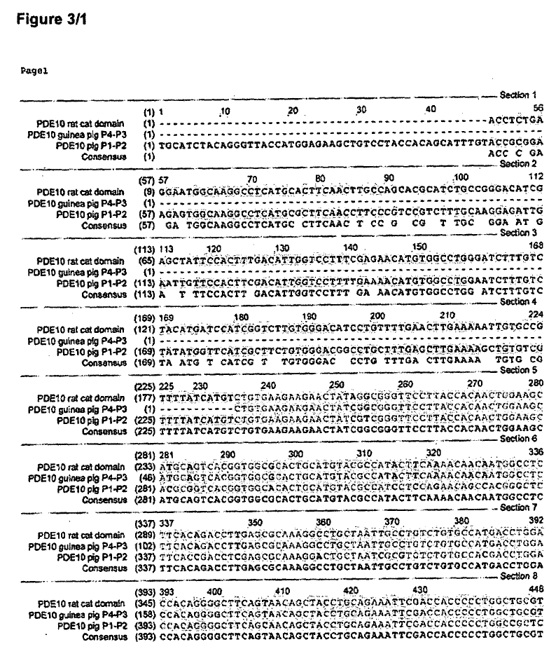

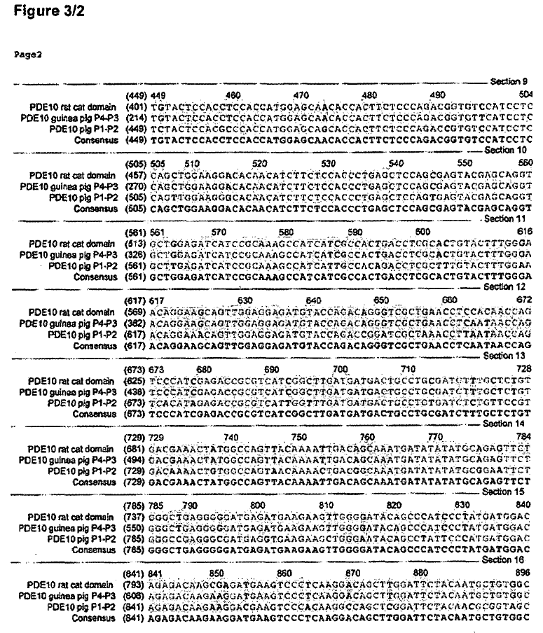

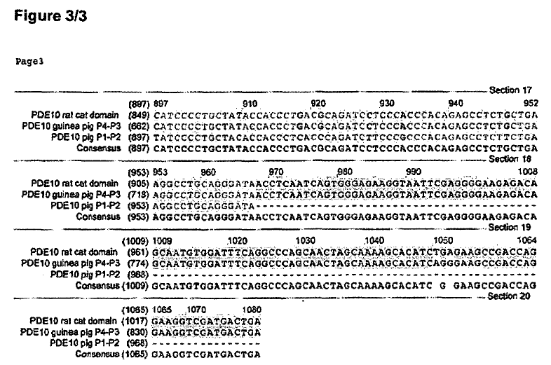

An alignment of the sequences showed a nearly complete accordance between the rat (published gene number NM-022236 3437 bp; coding sequence: 281-2665; catalytic domain 1634-2665) and the guinea pig. More differences were detected between rat and pig. For the alignment the coding areas are used only. The gene alignment is shown in FIG. 3.

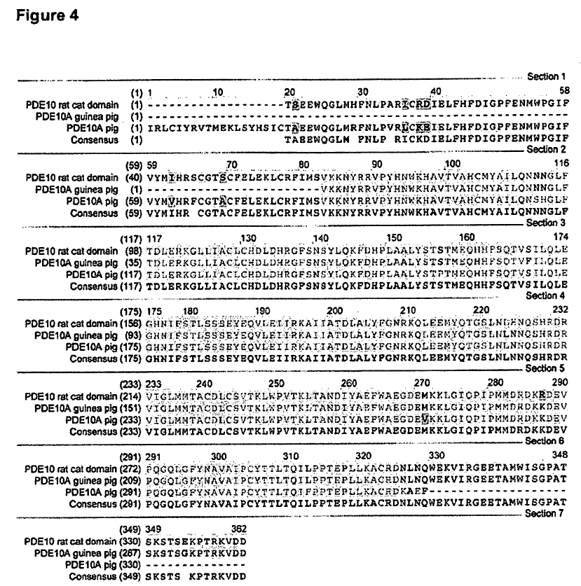

This results in the following differences in the protein sequences within the catalytic domain as shown in a protein alignment (FIG. 4).

PDE10 activity was determined in a one step procedure in microtiterplates. The reaction mixture of 100 μl contained 50 mM Tris-HCl/5 mM MgCl₂ buffer (pH=7.4) (Sigma, Deisenhofen, Germany; Merck, Darmstadt, Germany) 0.1 μM [³H]-cAMP (Amersham, Buckinghamshire, UK) and the enzyme. Nonspecific activity was tested without the enzyme. The reaction was initiated by addition of the substrate solution and was carried out at 37° C. for 30 minutes. Enzymatic activity was stopped by addition of 25 μl YSi-SPA beads (Amersham-Pharmacia). One hour later the mixture was measured in a liquid scintillation counter for microtiterplates (Microbeta Trilux). To pipette the incubation mixture a robot Biomek (Fa. Beckman) is used. The determined Km-values for the substrate cAMP is 78 nM for PDE10 from rat striatum, 88 nM for pig striatum and 66.7 nM for guinea pig striatum respectively. cGMP is the second substrate for PDE10, the Km values are 1800 nM, 2200 nM and 1700 nM for PDE10 from these species. For the test with cGMP 500 nM of this substrate was used. The optimal amount of enzyme in the assay has been determined and optimized for each enzyme preparation and substrate separately before using the enzyme in compound testing. For determination of IC₅₀ values the Hill-plot, 2-parameter-model, was used. Specific inhibitors of other PDE-Subtypes do not inhibit the PDE10 preparation significantly. Papaverine was used as the most common PDE10 inhibitor and inhibits the PDE10 with IC₅₀ values of 142 nM, 110 nM and 77 nM for PDE10 from striatum of rat, pig and guinea pig respectively.

See Table 7, Table 8, and Table 9 for PDE10 IC₅₀ values for select compounds of the invention.

### Example B

### Compound Data

The compounds of formula (I) are potent inhibitors of PDE10. A substance is considered to effectively inhibit PDE10 if it has an IC₅₀ of less than 10 μM, e.g., less than 1 μM. IC₅₀ values for select compounds are provided in Tables 7, 8, and 9, where “+” indicates that the IC₅₀ value is less than or equal to 10 nM; “++” indicates that the IC₅₀ value is between 10-100 nM; and “+++” indicates that the IC₅₀ value is equal to or greater than 100 nM.

TABLE 7
Analytical and assay data for select Examples

| | | | Human |
|---|---|---|---|
| | MS | PDE10 pig | PDE10 |
| Example | [M + H]⁺ | IC50 (nM) | (IC50 nM) |
| 1 | 413.1 | ++ | |
| 2 | 305.1 | + | |
| 3 | 285.1 | ++ | |
| 4 | 353 | + | |
| 5 | 323.2 | ++ | |
| 6 | 373.1 | ++ | |
| 7 | 373.1 | ++ | |
| 8 | 341.1 | ++ | |
| 9 | 324.1 | ++ | |
| 10 | 306.2 | ++ | |
| 11 | 306.2 | ++ | |
| 12 | 353 | + | |
| 13 | 353 | + | |
| 14 | 337.1 | + | |
| 15 | 353.1 | ++ | |
| 16 | 357.0 | + | |
| 17 | 353.1 | | ++ |
| 18 | 369.0 | + | |
| 19 | 369.0 | + | ++ |
| 20 | 407.0 | + | + |
| 21 | 337.1 | + | ++ |
| 22 | 357.0 | + | |
| 23 | 407.0 | + | + |
| 24 | 423.1 | ++ | ++ |
| 25 | 364.1 | + | ++ |
| 26 | 383.1 | ++ | ++ |
| 27 | 307.1 | + | ++ |
| 28 | 336.1 | ++ | |
| 29 | 336.1 | ++ | |
| 30 | 336.1 | ++ | |
| 31 | 338.1 | ++ | ++ |
| 32 | 320.1 | ++ | ++ |
| 33 | 338.1 | | ++ |
| 34 | 390.1 | | +++ |
| 35 | 427.0 | | ++ |
| 36 | 420.1 | | ++ |
| 37 | 373.1 | | + |
| 38 | 374.1 | | ++ |
| 39 | 452.0 | | + |
| 40 | 384 | | ++ |

TABLE 8
Analytical and assay data for select Examples

| | | MS | PDE10 pig |
|---|---|---|---|
| Example | Chemical Structure | [M + H]⁺ | IC50 (nM) |
| 41 | 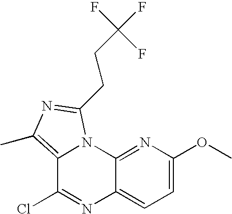 | 345 | ++ |
| 42 | 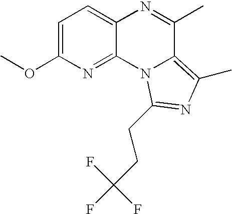 | 325 | ++ |
| 43 | 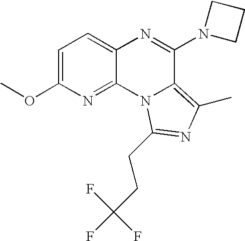 | 366.1 | +++ |
| 44 | 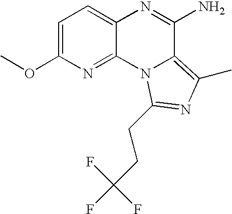 | 324.1 | ++ |
| 45 | 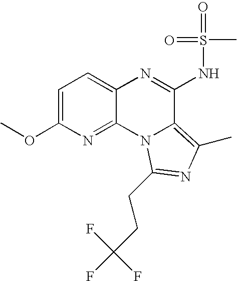 | 404.1 | ++ |
| 46 | 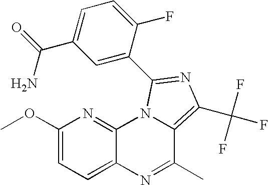 | 257.1 | +++ (Human PDE10) |
| 47 | 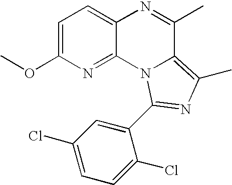 | 372.8 | + |
| 48 | 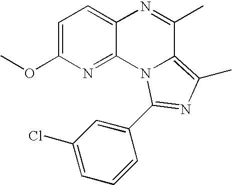 | 338.9 | + |
| 49 | 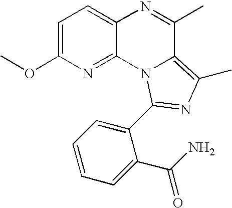 | 348.1 | +++ |
| 50 | 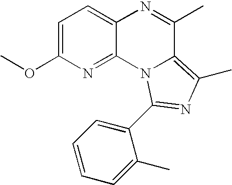 | 319.1 | + |
| 51 | 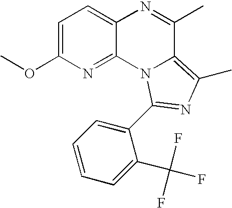 | 373.1 | ++ |
| 52 | 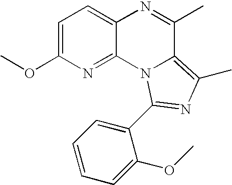 | 335.1 | ++ |
| 53 | 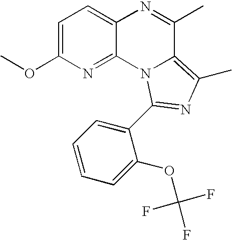 | 389.1 | ++ |
| 54 | 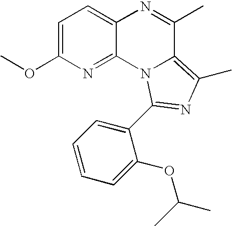 | 363.2 | +++ |
| 55 | 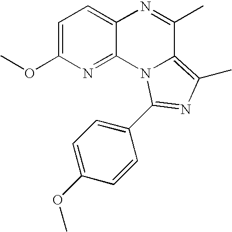 | 335.2 | ++ |
| 56 | 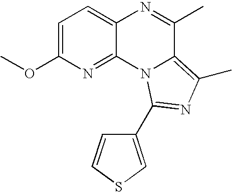 | 311.1 | ++ |
| 57 | 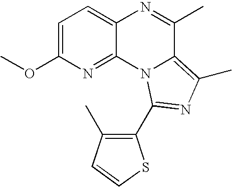 | 325.1 | + |
| 58 | 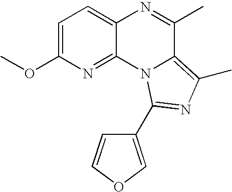 | 295.1 | ++ |
| 59 | 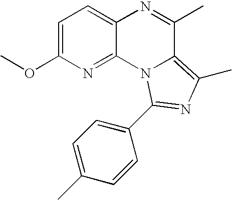 | 319.2 | ++ |
| 60 | 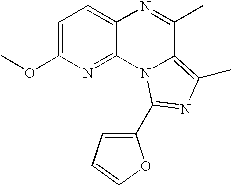 | 295.1 | ++ |
| 61 | 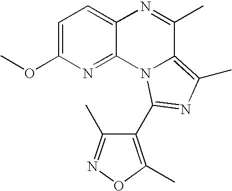 | 324.1 | ++ |
| 62 | 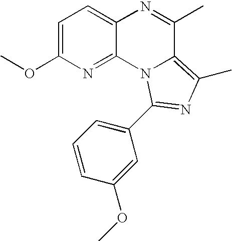 | 335.2 | + |
| 63 | 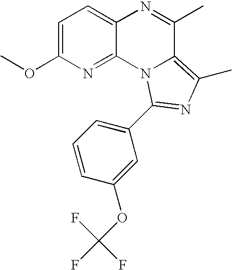 | 389.2 | ++ |
| 64 | 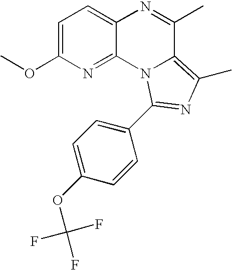 | 389.2 | ++ |
| 65 | 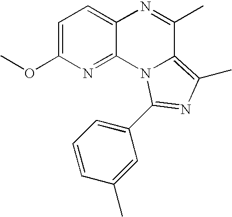 | 319.1 | + |
| 66 | 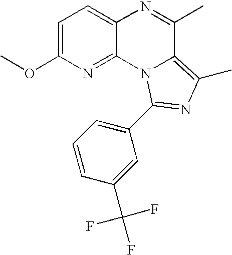 | 373.1 | ++ |
| 67 | 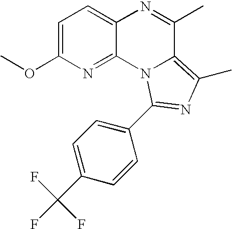 | 373.1 | ++ |
| 68 | 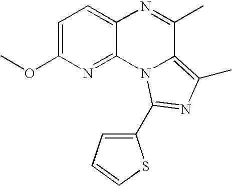 | 311.1 | ++ |
| 69 | 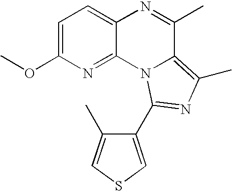 | 325.1 | + |
| 70 | 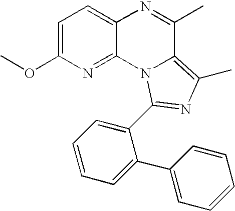 | 381.1 | ++ |
| 71 | 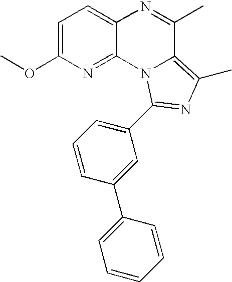 | 381.2 | ++ |
| 72 | 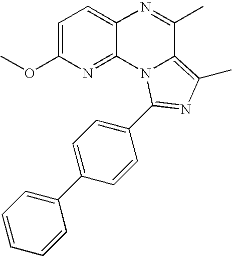 | 381.2 | +++ |
| 73 | 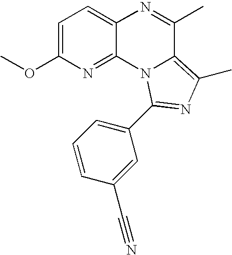 | 330.1 | ++ |
| 74 | 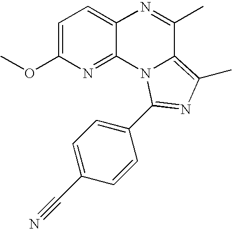 | 330.2 | ++ |
| 75 | 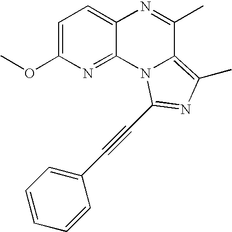 | 329.1 | ++ |
| 76 | 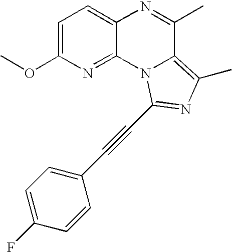 | 347.1 | +++ |
| 77 | 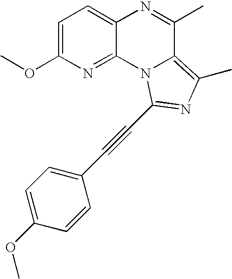 | 359.1 | ++ |
| 78 | 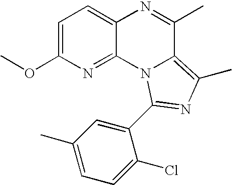 | 353.1 | + |
| 79 | 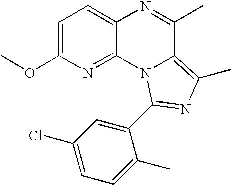 | 353.1 | + |
| 80 | 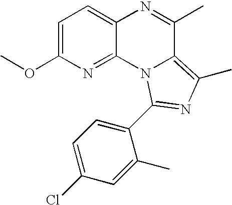 | 353 | + |
| 81 | 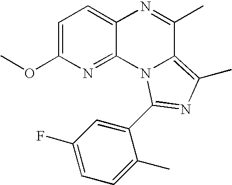 | 337.1 | + |
| 82 | 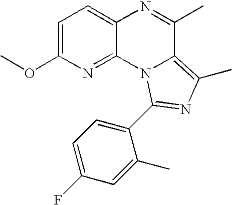 | 337.1 | + |
| 83 | 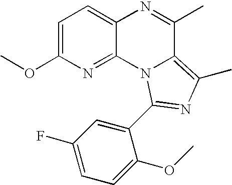 | 353.1 | ++ |
| 84 | 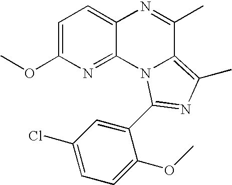 | 369 | ++ |
| 85 | 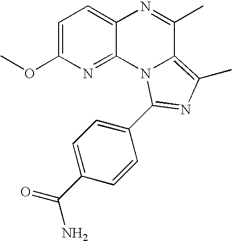 | 348.1 | + |
| 86 | 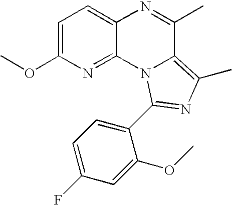 | 353.1 | ++ |
| 87 | 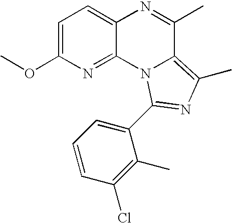 | 353 | + |
| 88 | 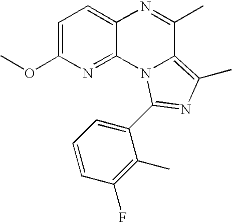 | 337.1 | + |
| 89 | 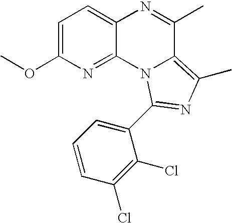 | 373 | + |
| 90 | 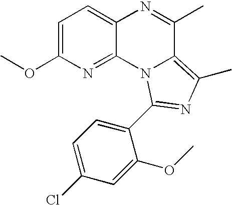 | 369.2 | ++ |
| 91 | 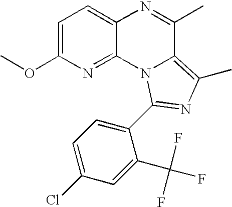 | 407.2 | + |
| 92 | 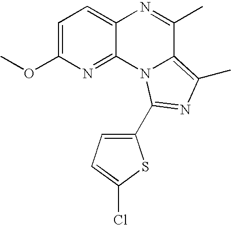 | 345.2 | ++ |
| 93 | 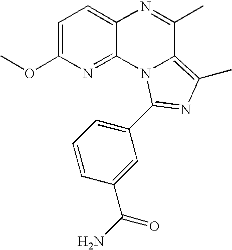 | 348.1 | + |
| 94 | 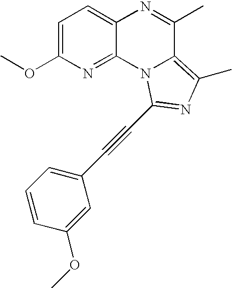 | 359.1 | +++ |
| 95 | 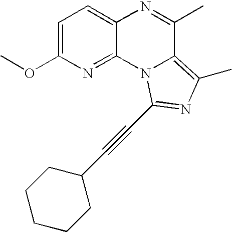 | 335.1 | +++ |
| 96 | 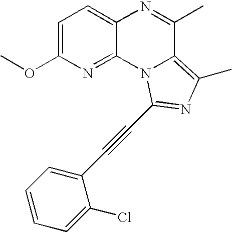 | 363.1 | ++ |
| 97 | 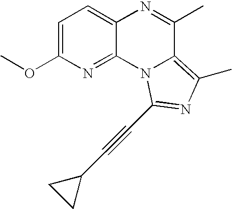 | 293.1 | ++ |
| 98 | 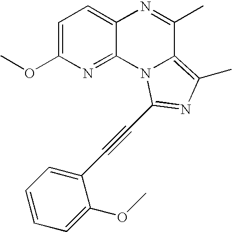 | 359.1 | +++ |
| 99 | 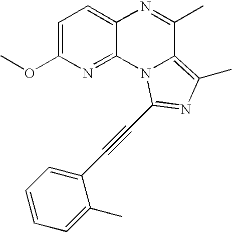 | 343.1 | +++ |
| 100 | 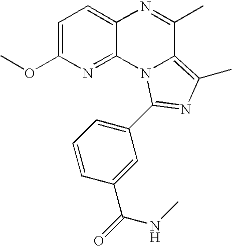 | 362.1 | +++ |
| 101 | 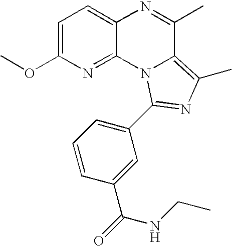 | 376.1 | ++ |
| 102 | 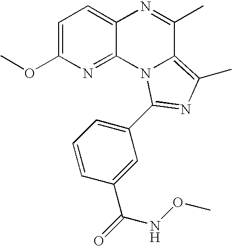 | 378 | ++ |
| 103 | 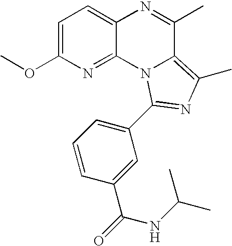 | 390.1 | ++ |
| 104 | 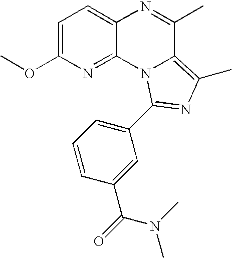 | 376.1 | +++ |
| 105 | 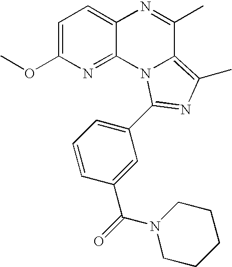 | 416.1 | +++ |
| 106 | 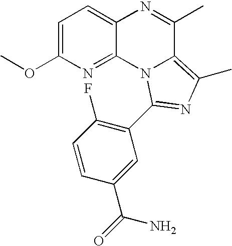 | 366.1 | + |
| 107 | 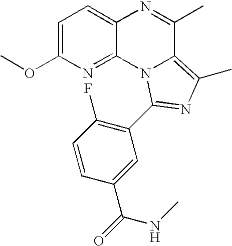 | 380 | +++ |

TABLE 9
Analytical and assay data for select Examples

| | MS | hPDE10 |
|---|---|---|
| Example | [M + H]⁺ | IC50 (nM) |
| 129 | 366.1 | + |
| 130 | 366.1 | + |
| 131 | 382.1 | ++ |
| 132 | 382.1 | + |
| 133 | 268.1 | +++ |
| 134 | 393.2 | ++ |
| 135 | 377.2 | ++ |
| 136 | 377.2 | + |
| 137 | 447.1 | ++ |
| 138 | 397.1 | + |
| 139 | 376.2 | +++ |
| 140 | 360.2 | ++ |
| 141 | 378.2 | + |
| 142 | 388.2 | + |
| 143 | 388.2 | + |
| 144 | 406.2 | + |
| 145 | 406.2 | + |
| 146 | 403.2 | +++ |
| 147 | 403.1 | +++ |
| 148 | 424.1 | +++ |
| 149 | 402.1 | + |
| 150 | 402.1 | + |
| 151 | 406.1 | + |
| 152 | 305.1 | ++ |
| 153 | 369.2 | + |
| 154 | 351.1 | + |
| 155 | 387.1 | ++ |
| 123 | 359.2 | + |
| 156 | 343.1 | |
| 157 | 413.1 | ++ |
| 158 | 396.1 | + |
| 159 | 306.1 | +++ |
| 160 | 320.1 | + |
| 161 | 336.1 | ++ |
| 162 | 320.2 | ++ |
| 163 | 320.1 | + |
| 164 | 317 | |
| 165 | 330.1 | ++ |
| 166 | 377 | + |
| 167 | 359.1 | ++ |
| 168 | 377.1 | + |
| 169 | 331.1 | +++ |
| 170 | 317.1 | +++ |
| 171 | 317.1 | ++ |
| 172 | 349.1 | +++ |
| 111 | 350 | + (pPDE10) |
| 112 | 384 | + (pPDE10) |
| 173 | 323.1 | ++ |
| 174 | 324.1 | ++ |
| 175 | 324.1 | + |
| 176 | 340.1 | + |
| 177 | 344.1 | + |
| 178 | 349.2 | +++ |
| 179 | 293.1 | ++ |
| 180 | 309.1 | ++ |
| 181 | 294.1 | ++ |
| 182 | 351.2 | +++ |
| 183 | 340.1 | + |
| 184 | 336.1 | + |
| 185 | 308.1 | ++ |
| 186 | 340.1 | + |
| 187 | 391.2 | +++ |
| 188 | 390.2 | +++ |
| 189 | 337.2 | +++ |
| 190 | 408.2 | +++ |
| 191 | 337.2 | + |

Various modifications of the invention, in addition to those described herein, will be apparent to those skilled in the art from the foregoing description. Such modifications are also intended to fall within the scope of the appended claims. Each reference, including all patents, patent applications, and journal literature, cited in the present application is incorporated herein by reference in its entirety.

What is claimed is:

1. A compound of Formula (I): 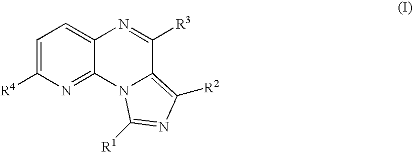
wherein:
R¹ is:
C₁₋₈ alkyl, C₂₋₈ alkenyl, or C₂₋₈ alkynyl, each optionally mono- or polysubstituted with substituents independently selected from halo, OH, O—C₁₋₃ alkyl, cyano, and a cyclic radical;
aryl, heteroaryl, C₃₋₈ cyclo(hetero)alkyl, aryl-C₁₋₅ alkyl, or heteroaryl-C₁₋₅ alkyl, each optionally mono- or polysubstituted with substituents independently selected from halo, amino, C₁₋₃ alkylamino, di-C₁₋₃ alkylamino, nitro, C₁₋₅ alkyl, O—C₁₋₃ alkyl, cyano, C₁₋₃ haloalkyl, O—C₁₋₃ haloalkyl, COOH, —(C═O)—NR⁶R⁷, SO₂NR⁶R⁷, a cyclic radical, and C₃₋₈ cyclo(hetero)alkyl; or two adjacent O—C₁₋₃ alkyl groups, together with the atoms to which they are attached, form a 5-7 membered cycloheteroalkyl group;
R² is C₁₋₈ alkyl, C₃₋₈ cyclo(hetero)alkyl, aryl-C₁₋₅ alkyl, or heteroaryl-C₁₋₅ alkyl, each optionally mono- or polysubstituted with substituents independently selected from halo, OH, O—C₁₋₃ alkyl, and a cyclic radical;
R³ is:
cyano;
C₁₋₈ alkyl, C₁₋₈ haloalkyl, C₃₋₈ cyclo(hetero)alkyl, aryl-C₁₋₅ alkyl, heteroaryl-C₁₋₅ alkyl, each optionally mono- or polysubstituted with substituents independently selected from halo, OH, O—C₁₋₃ alkyl, and a cyclic radical;
NR⁶R⁷, (CO)OR⁶, (CO)NR⁶R⁷, NR⁵(CO)OR⁶, NR⁵(CO)R⁶, NR⁵(C═O)—NR⁶R⁷, or NR⁵(SO₂R⁶), wherein R⁵, R⁶, and R⁷ are independently selected from H, a cyclic radical, C₁₋₈ alkyl, O—C₁₋₅ alkyl, C₃₋₆ cycloalkyl, aryl-C₁₋₅ alkyl, and heteroaryl-C₁₋₅ alkyl, wherein C₁₋₈ alkyl, O—C₁₋₅ alkyl, C₃₋₆ cycloalkyl, aryl-C₁₋₅ alkyl, and heteroaryl-C₁₋₅ alkyl are optionally mono- or polysubstituted with substituents independently selected from halo, OH, O—C₁₋₃ alkyl, and a cyclic radical;
or R⁶ and R⁷, together with the nitrogen atom to which they are attached, form a 4-7 membered cycloheteroalkyl group; and
R⁴ is halo, R⁸, or OR⁸,
wherein R⁸ is:
H,
C₁₋₈ alkyl or C₃₋₆ cyclo(hetero)alkyl, each optionally mono- or polysubstituted with substituents independently selected from halo, OH, O—C₁₋₃ alkyl, C₂₋₈ alkynyl, and a cyclic radical;
aryl-C₁₋₅ alkyl or heteroaryl-C₁₋₅ alkyl, each optionally mono- or polysubstituted with substituents independently selected from halo, amino, C₁₋₃ alkylamino, di-C₁₋₃ alkylamino, nitro, C₁₋₃ alkyl, O—C₁₋₃ alkyl, and a cyclic radical;
or an N-oxide thereof, or a pharmaceutically acceptable salt thereof.

2. The compound of claim 1, or N-oxide thereof, or pharmaceutically acceptable salt thereof, wherein R¹ is C₁₋₈ alkyl optionally mono- or polysubstituted with halo.

3. The compound of claim 2, or N-oxide thereof, or pharmaceutically acceptable salt thereof, wherein R¹ is propyl optionally mono- or polysubstituted with fluoro.

4. The compound of claim 1, or N-oxide thereof, or pharmaceutically acceptable salt thereof, wherein R¹ is C₂₋₈ alkynyl optionally mono- or polysubstituted with a cyclic radical.

5. The compound of claim 4, or N-oxide thereof, or pharmaceutically acceptable salt thereof, wherein R¹ is C₂ alkynyl monosubstituted with C₃₋₈ cycloalkyl.

6. The compound of claim 5, or N-oxide thereof, or pharmaceutically acceptable salt thereof, wherein R¹ is C₂ alkynyl monosubstituted with cyclopropyl or cyclohexyl.

7. The compound of claim 1, or N-oxide thereof, or pharmaceutically acceptable salt thereof, wherein R¹ is C₂ alkynyl monosubstituted with aryl, and said aryl is optionally mono- or polysubstituted with substituents independently selected from halo, C₁₋₃ alkyl, O—C₁₋₃ alkyl, cyano, and C₁₋₃ haloalkyl.

8. The compound of claim 7, or N-oxide thereof, or pharmaceutically acceptable salt thereof, wherein R¹ is C₂ alkynyl monosubstituted with phenyl optionally mono- or polysubstituted with substituents independently selected from fluoro, methyl, and OCH₃.

9. The compound of claim 1, or N-oxide thereof, or pharmaceutically acceptable salt thereof, wherein R¹ is aryl or heteroaryl, each optionally mono- or polysubstituted with substituents independently selected from halo, amino, C₁₋₃ alkylamino, di-C₁₋₃ alkylamino, nitro, C₁₋₃ alkyl, O—C₁₋₃ alkyl, cyano, C₁₋₃ haloalkyl, O—C₁₋₃ haloalkyl, —(C═O)—NR⁶R⁷, and a cyclic radical.

10. The compound of claim 1, or N-oxide thereof, or pharmaceutically acceptable salt thereof, wherein R¹ is aryl optionally mono- or polysubstituted with substituents independently selected from halo, C₁₋₃ alkyl, O—C₁₋₃ alkyl, cyano, C₁₋₃ haloalkyl, O—C₁₋₃ haloalkyl, —(C═O)—NR⁶R⁷, and a cyclic radical.

11. The compound of claim 10, or N-oxide thereof, or pharmaceutically acceptable salt thereof, wherein R¹ is aryl mono-substituted with a cyclic radical.

12. The compound of claim 11, or N-oxide thereof, or pharmaceutically acceptable salt thereof, wherein R¹ is aryl mono-substituted with phenyl.

13. The compound of claim 11, or N-oxide thereof, or pharmaceutically acceptable salt thereof, wherein R¹ is aryl mono-substituted with morpholino.

14. The compound of claim 10, or N-oxide thereof, or pharmaceutically acceptable salt thereof, wherein R¹ is aryl mono-substituted with —(C═O)—NR⁶R⁷, and said R⁶ and R⁷ are independently selected from H, C₁₋₈ alkyl, and O—C₁₋₅ alkyl.

15. The compound of claim 14, or N-oxide thereof, or pharmaceutically acceptable salt thereof, wherein R¹ is aryl mono-substituted with —(C═O)—NR⁶R⁷, and R⁶ and R⁷ are independently selected from H, methyl, and OCH₃.

16. The compound of claim 15, or N-oxide thereof, or pharmaceutically acceptable salt thereof, wherein R¹ is aryl mono-substituted with —(C═O)—NR⁶R⁷, and said R⁶ and R⁷ together with the nitrogen atom to which they are attached form a 5-6 membered cycloheteroalkyl group.

17. The compound of claim 1, or N-oxide thereof, or pharmaceutically acceptable salt thereof, wherein R¹ is aryl optionally mono- or polysubstituted with substituents independently selected from COOH and SO₂NR⁷.

18. The compound of claim 17, or N-oxide thereof, or pharmaceutically acceptable salt thereof, wherein R¹ is aryl optionally mono- or polysubstituted with substituents independently selected from COOH and SO₂NH₂.

19. The compound of claim 1, or N-oxide thereof, or pharmaceutically acceptable salt thereof, wherein R¹ is 5- or 6-membered heteroaryl optionally mono- or polysubstituted with substituents independently selected from halo, C₁₋₅ alkyl, amino, C₁₋₃ alkylamino, di-C₁₋₃ alkylamino, O—C₁₋₃ alkyl, cyano, C₁₋₃ haloalkyl, and a cyclic radical.

20. The compound of claim 19, or N-oxide thereof, or pharmaceutically acceptable salt thereof, wherein R¹ is 5- or 6-membered heteroaryl optionally mono- or polysubstituted with substituents independently selected from halo, C₁₋₃ alkyl, cyano, and C₁₋₃ haloalkyl.

21. The compound of claim 19, or N-oxide thereof, or pharmaceutically acceptable salt thereof, wherein R¹ is 5-membered heteroaryl optionally mono- or polysubstituted with substituents independently selected from amino, C₁₋₃ alkylamino, di-C₁₋₃ alkylamino, O—C₁₋₃ alkyl, and a cyclic radical.

22. The compound of claim 20, or N-oxide thereof, or pharmaceutically acceptable salt thereof, wherein R¹ is 5-membered heteroaryl optionally mono- or polysubstituted with substituents independently selected from halo, C₁₋₃ alkyl, cyano, and C₁₋₃ haloalkyl.

23. The compound of claim 22, or N-oxide thereof, or pharmaceutically acceptable salt thereof, wherein R¹ is furan or thiophene.

24. The compound of claim 22, or N-oxide thereof, or pharmaceutically acceptable salt thereof, wherein R¹ is pyrrole or pyrazole, each optionally mono- or polysubstituted with substituents independently selected from halo, C₁₋₃ alkyl, cyano, and C₁₋₃ haloalkyl.

25. The compound of claim 24, or N-oxide thereof, or pharmaceutically acceptable salt thereof, wherein R¹ is pyrazole optionally mono- or polysubstituted with C₁₋₅ alkyl.

26. The compound of claim 25, or N-oxide thereof, or pharmaceutically acceptable salt thereof, wherein R¹ is pyrazole mono-substituted with methyl.

27. The compound of claim 25, or N-oxide thereof, or pharmaceutically acceptable salt thereof, wherein R¹ is pyrazole polysubstituted with methyl.

28. The compound of claim 27, or N-oxide thereof, or pharmaceutically acceptable salt thereof, wherein R¹ is 1,3,5-trimethyl-1H-pyrazole-4-yl.

29. The compound of claim 27, or N-oxide thereof, or pharmaceutically acceptable salt thereof, wherein R¹ is 3,5-dimethyl-1H-pyrazole-4-yl.

30. The compound of claim 1, or N-oxide thereof, or pharmaceutically acceptable salt thereof, wherein R¹ is 6-membered heteroaryl optionally mono- or polysubstituted with substituents independently selected from halo, C₁₋₅ alkyl, amino, C₁₋₃ alkylamino, di-C₁₋₃ alkylamino, O—C₁₋₃ alkyl, cyano, C₁₋₃ haloalkyl, and a cyclic radical.

31. The compound of claim 30, or N-oxide thereof, or pharmaceutically acceptable salt thereof, wherein R¹ is pyridine or pyrimidine, each optionally mono- or polysubstituted with substituents independently selected from amino, C₁₋₃ alkylamino, di-C₁₋₃ alkylamino, O—C₁₋₃ alkyl, and a cyclic radical.

32. The compound of claim 30, or N-oxide thereof, or pharmaceutically acceptable salt thereof, wherein R¹ is pyridine or pyrimidine, each optionally mono- or polysubstituted with substituents independently selected from halo, C₁₋₅ alkyl, cyano, and C₁₋₃ haloalkyl.

33. The compound of claim 32, or N-oxide thereof, or pharmaceutically acceptable salt thereof, wherein R¹ is pyridine optionally mono- or polysubstituted with substituents independently selected from halo and C₁₋₅ alkyl.

34. The compound of claim 33, or N-oxide thereof, or pharmaceutically acceptable salt thereof, wherein R¹ is pyridine optionally mono- or polysubstituted with substituents independently selected from fluoro, chloro, and methyl.

35. The compound of claim 33, or N-oxide thereof, or pharmaceutically acceptable salt thereof, wherein R¹ is pyridine mono-substituted with methyl.

36. The compound of claim 35, or N-oxide thereof, or pharmaceutically acceptable salt thereof, wherein R¹ is 4-methylpyridin-3-yl or 2-methylpyridin-3-yl.

37. The compound of claim 31, or N-oxide thereof, or pharmaceutically acceptable salt thereof, wherein R¹ is pyridine optionally mono-substituted with di-methylamino, OCH₃, or morpholino.

38. The compound of claim 1, or N-oxide thereof, or pharmaceutically acceptable salt thereof, wherein R² is C₁₋₈ alkyl optionally mono- or polysubstituted with halo.

39. The compound of claim 38, or N-oxide thereof, or pharmaceutically acceptable salt thereof, wherein R² is methyl.

40. The compound of claim 1, or N-oxide thereof, or pharmaceutically acceptable salt thereof, wherein R³ is C₁₋₈ alkyl, C₁₋₈ haloalkyl, C₃₋₈ cyclo(hetero)alkyl, aryl-C₁₋₅ alkyl, or heteroaryl-C₁₋₅ alkyl, each optionally mono- or polysubstituted with substituents independently selected from halo, OH, O—C₁₋₃ alkyl, and a cyclic radical.

41. The compound of claim 40, or N-oxide thereof, or pharmaceutically acceptable salt thereof, wherein R³ is C₁₋₈ alkyl or C₁₋₈ haloalkyl.

42. The compound of claim 41, or N-oxide thereof, or pharmaceutically acceptable salt thereof, wherein R³ is CH₃.

43. The compound of claim 1, or N-oxide thereof, or pharmaceutically acceptable salt thereof, wherein R³ is cyano.

44. The compound of claim 1, or N-oxide thereof, or pharmaceutically acceptable salt thereof, wherein R⁴ is OR⁸, and said R⁸ is C₁₋₈ alkyl optionally mono- or polysubstituted substituents independently selected from with halo, OH, O—C₁₋₃ alkyl, and a cyclic radical.

45. The compound of claim 44, or N-oxide thereof, or pharmaceutically acceptable salt thereof, wherein R⁴ is OR⁸, and said R⁸ is methyl optionally mono- or polysubstituted with substituents independently selected from halo, OH, O—C₁₋₃ alkyl, and a cyclic radical.

46. The compound of claim 45, or N-oxide thereof, or pharmaceutically acceptable salt thereof, wherein R⁴ is OCH₃.

47. The compound of claim 45, or N-oxide thereof, or pharmaceutically acceptable salt thereof, wherein R⁴ is OR⁸, and said R⁸ is methyl mono- or polysubstituted with cyclopropyl.

48. The compound of claim 44, or N-oxide thereof, or pharmaceutically acceptable salt thereof, wherein R⁴ is OR⁸, and said R⁸ is ethyl optionally mono- or polysubstituted with halo.

49. The compound of claim 48, or N-oxide thereof, or pharmaceutically acceptable salt thereof, wherein R⁴ is OCH₂CH₂F, OCH₂CHF₂, or OCH₂CF₃.

50. The compound of claim 1, or N-oxide thereof, or pharmaceutically acceptable salt thereof, wherein R⁴ is OR⁸, wherein said R⁸ is aryl-C₁₋₅ alkyl or heteroaryl-C₁₋₅ alkyl, each optionally mono- or polysubstituted with substituents independently selected from halo, C₁₋₃ alkyl, and O—C₁₋₃ alkyl.

51. The compound of claim 50, or N-oxide thereof, or pharmaceutically acceptable salt thereof, wherein R⁸ is benzyl optionally mono- or polysubstituted with fluoro.

52. The compound of claim 50, or N-oxide thereof, or pharmaceutically acceptable salt thereof, wherein R⁸ is pyridinyl.

53. The compound of claim 1, or N-oxide thereof, or pharmaceutically acceptable salt thereof, wherein the compound has Formula (I): 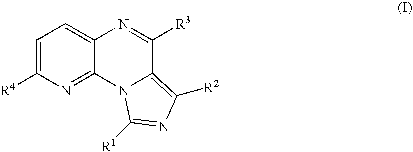
wherein:
R¹ is:
C₁₋₈ alkyl, C₂₋₈ alkenyl, or C₂₋₈ alkynyl, each optionally mono- or polysubstituted with substituents independently selected from halo, cyano, and a cyclic radical;
aryl, heteroaryl, C₃₋₈ cyclo(hetero)alkyl, aryl-C₁₋₅ alkyl, or heteroaryl-C₁₋₅ alkyl, each optionally mono- or polysubstituted with substituents independently selected from halo, amino, C₁₋₃ alkylamino, di-C₁₋₃ alkylamino, nitro, C₁₋₅ alkyl, O—C₁₋₃ alkyl, cyano, C₁₋₃ haloalkyl, O—C₁₋₃ haloalkyl, COOH, —(C═O)—NR⁶R⁷, SO₂NR⁶R⁷, and a cyclic radical; or two O—C 3 alkyl groups, together with the atoms to which they are attached, form a 5-7 membered cycloheteroalkyl group;
R² is C₁₋₈ alkyl optionally mono- or polysubstituted with substituents independently selected from halo and a cyclic radical;
R³ is:
cyano;
C₁₋₈ alkyl or C₁₋₈ haloalkyl, each optionally mono- or polysubstituted with substituents independently selected from halo, OH, O—C₁₋₃ alkyl, and a cyclic radical;
(CO)NR⁶R⁷, wherein R⁶ and R⁷ are selected from H, a cyclic radical, C₁₋₈ alkyl, O—C₁₋₅ alkyl; or R⁶ and R⁷, together with the nitrogen atom to which they are attached, form a 4-7 membered cycloheteroalkyl group; and
R⁴ is R⁸ or OR⁸, wherein R⁸ is C₁₋₈ alkyl optionally mono- or polysubstituted with substituents independently selected from halo, OH, O—C₁₋₃ alkyl, C₂₋₈ alkynyl, and a cyclic radical.

54. The compound of claim 1, or N-oxide thereof, or pharmaceutically acceptable salt thereof, wherein:
R¹ is:
C₁₋₈ alkyl, C₂₋₈ alkenyl, or C₂₋₈ alkynyl, each optionally mono- or polysubstituted with substituents independently selected from halo and a cyclic radical;
aryl, heteroaryl, C₃₋₈ cyclo(hetero)alkyl, aryl-C₁₋₅ alkyl, or heteroaryl-C₁₋₅ alkyl, each optionally mono- or polysubstituted with substituents independently selected from halo, amino, C₁₋₃ alkylamino, di-C₁₋₃ alkylamino, nitro, C₁₋₃ alkyl, O—C₁₋₃ alkyl, cyano, C₁₋₃ haloalkyl, O—C₁₋₃ haloalkyl, —(C═O)—NR⁶R⁷, and a cyclic radical; or two O—C₁₋₃ alkyl groups, together with the atoms to which they are attached, form a 5-7 membered cycloheteroalkyl group;
R² is C₁₋₈ alkyl optionally mono- or polysubstituted with substituents independently selected from halo and a cyclic radical;
R³ is:
cyano;
C₁₋₈ alkyl or C₁₋₈ haloalkyl each optionally mono- or polysubstituted with halo, OH, O—C₁₋₃ alkyl, or a cyclic radical;
(CO)NR⁶R⁷, wherein R⁶ and R⁷ are selected from H, a cyclic radical, C₁₋₈ alkyl, O—C₁₋₅ alkyl; or R⁶ and R⁷, together with the nitrogen atom to which they are attached, form a 4-7 membered cycloheteroalkyl group; and
R⁴ is R⁸ or OR⁸, wherein R⁸ is C₁₋₈ alkyl optionally mono- or polysubstituted with substituents independently selected from halo, OH, O—C₁₋₃ alkyl, and a cyclic radical.

55. The compound of claim 1, or N-oxide thereof, or pharmaceutically acceptable salt thereof, wherein:
R¹ is aryl or heteroaryl, each optionally mono- or polysubstituted with substituents independently selected from halo, C₁₋₃ alkyl, and O—C₁₋₃ alkyl;
each of R² and R³ is independently C₁₋₈ alkyl; and
R⁴ is C₁₋₈ alkyl or O—C₁₋₈ alkyl.

56. A compound of Formula (I): 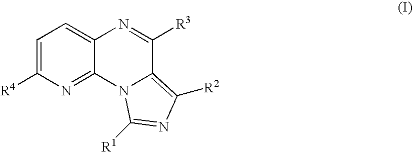
wherein:
R¹ is:
C₁₋₈ alkyl, C₂₋₈ alkenyl, C₂₋₈ alkynyl, each optionally mono- or polysubstituted with substituents independently selected from halo, OH, O—C₁₋₃ alkyl, and a cyclic radical;
aryl, heteroaryl, C₃₋₈ cyclo(hetero)alkyl, aryl-C₁₋₅ alkyl, or heteroaryl-C₁₋₅ alkyl, each optionally mono- or polysubstituted with substituents independently selected from halo, amino, C₁₋₃ alkylamino, di-C₁₋₃ alkylamino, nitro, C₁₋₃ alkyl, O—C₁₋₃ alkyl, cyano, C₁₋₃ haloalkyl, O—C₁₋₃ haloalkyl, —(C═O)—NR⁶R⁷, and a cyclic radical; or two adjacent O—C₁₋₃ alkyl groups, together with the atoms to which they are attached, form a 5-7 membered cycloheteroalkyl group; and
R² is C₁₋₈ alkyl, C₃₋₈ cyclo(hetero)alkyl, aryl-C₁₋₅ alkyl, or heteroaryl-C₁₋₅ alkyl, each optionally mono- or polysubstituted with substituents independently selected from halo, OH, O—C₁₋₃ alkyl, and a cyclic radical;
R³ is:
cyano;
C₁₋₈ alkyl, C₁₋₈ haloalkyl, C₃₋₈ cyclo(hetero)alkyl, aryl-C₁₋₅ alkyl, heteroaryl-C₁₋₅ alkyl, each optionally mono- or polysubstituted with substituents independently selected from halo, OH, O—C₁₋₃ alkyl, and a cyclic radical;
NR⁶R⁷, (CO)OR⁶, (CO)NR⁶R⁷, NR⁵(CO)OR⁶, NR⁵(CO)R⁶, NR⁵(C═O)—NR⁶R⁷, or NR⁵(SO₂R⁶), wherein R⁵, R⁶, and R⁷ are independently selected from H, a cyclic radical, C₁₋₈ alkyl, O—C₁₋₅ alkyl, C₃₋₆ cycloalkyl, aryl-C₁₋₅ alkyl, and heteroaryl-C₁₋₅ alkyl, wherein said C₁₋₈ alkyl, O—C₁₋₅ alkyl, C₃₋₆ cycloalkyl, aryl-C₁₋₅ alkyl, and heteroaryl-C₁₋₅ alkyl are optionally mono- or polysubstituted with substituents independently selected from halo, OH, O—C₁₋₃ alkyl, and a cyclic radical;
or R⁶ and R⁷, together with the nitrogen atom to which they are attached, form a 4-7 membered cycloheteroalkyl group; and
R⁴ is halo, R⁸, or OR⁸,
wherein R⁸ is:
H,
C₁₋₈ alkyl or C₃₋₆ cyclo(hetero)alkyl, each optionally mono- or polysubstituted with substituents independently selected from halo, OH, O—C₁₋₃ alkyl, and a cyclic radical;
aryl-C₁₋₅ alkyl or heteroaryl-C₁₋₅ alkyl, each optionally mono- or polysubstituted with substituents independently selected from halo, amino, C₁₋₃ alkylamino, di-C₁₋₃ alkylamino, nitro, C₁₋₃ alkyl, O—C₁₋₃ alkyl, and a cyclic radical;
or an N-oxide thereof, or a pharmaceutically acceptable salt thereof.

57. The compound of claim 56, or N-oxide thereof, or pharmaceutically acceptable salt thereof, wherein:
R³ is:
cyano;
C₁₋₈ alkyl, C₁₋₈ haloalkyl, C₃₋₈ cyclo(hetero)alkyl, aryl-C₁₋₅ alkyl, heteroaryl-C₁₋₅ alkyl, each optionally mono- or polysubstituted with substituents independently selected from halo, OH, O—C₁₋₃ alkyl, and a cyclic radical;
(CO)OR⁶ or (CO)NR⁶R⁷, wherein R⁵, R⁶, and R⁷ are independently selected from H, a cyclic radical, C₁₋₈ alkyl, O—C₁₋₅ alkyl, C₃₋₆ cycloalkyl, aryl-C₁₋₅ alkyl, and heteroaryl-C₁₋₅ alkyl, wherein C₁₋₈ alkyl, O—C₁₋₅ alkyl, C₃₋₆ cycloalkyl, aryl-C₁₋₅ alkyl, and heteroaryl-C₁₋₅ alkyl are optionally mono- or polysubstituted substituents independently selected from with halo, OH, O—C₁₋₃ alkyl, and a cyclic radical;
or R⁶ and R⁷, together with the nitrogen atom to which they are attached, form a 4-7 membered cycloheteroalkyl group.

58. The compound of claim 1 selected from:
2-ethoxy-6,7-dimethyl-9-propylimidazo[1,5-a]pyrido[3,2-e]pyrazine;
9-(2-chlorophenyl)-2-ethoxy-6,7-dimethylimidazo[1,5-a]pyrido[3,2-e]pyrazine;
9-(3-fluorophenyl)-2-methoxy-6,7-dimethylimidazo[1,5-a]pyrido[3,2-e]pyrazine;
9-(3,5-dichlorophenyl)-2-methoxy-6,7-dimethylimidazo[1,5-a]pyrido[3,2-e]pyrazine;
9-(3,4-dichlorophenyl)-2-methoxy-6,7-dimethylimidazo[1,5-a]pyrido[3,2-e]pyrazine;
9-(2,4-difluorophenyl)-2-methoxy-6,7-dimethylimidazo[1,5-a]pyrido[3,2-e]pyrazine;
9-(6-fluoropyridin-3-yl)-2-methoxy-6,7-dimethylimidazo[1,5-a]pyrido[3,2-e]pyrazine;
2-methoxy-6,7-dimethyl-9-pyridin-3-ylimidazo[1,5-a]pyrido[3,2-e]pyrazine;
2-methoxy-6,7-dimethyl-9-pyridin-4-ylimidazo[1,5-a]pyrido[3,2-e]pyrazine;
9-(2-chloro-4-methylphenyl)-2-methoxy-6,7-dimethylimidazo[1,5-a]pyrido[3,2-e]pyrazine;
9-(4-chloro-2-methylphenyl)-2-methoxy-6,7-dimethylimidazo[1,5-a]pyrido[3,2-e]pyrazine;
9-(2-fluoro-4-methylphenyl)-2-methoxy-6,7-dimethylimidazo[1,5-a]pyrido[3,2-e]pyrazine;
9-(2-fluoro-3-methoxyphenyl)-2-methoxy-6,7-dimethylimidazo[1,5-a]pyrido[3,2-e]pyrazine;
9-(2-chloro-4-fluorophenyl)-2-methoxy-6,7-dimethylimidazo[1,5-a]pyrido[3,2-e]pyrazine;
9-(4-chloro-2-fluorophenyl)-2-methoxy-6,7-dimethylimidazo[1,5-a]pyrido[3,2-e]pyrazine;
9-(2-chloro-4-methoxyphenyl)-2-methoxy-6,7-dimethylimidazo[1,5-a]pyrido[3,2-e]pyrazine;
9-(2-chloro-5-methoxyphenyl)-2-methoxy-6,7-dimethylimidazo[1,5-a]pyrido[3,2-e]pyrazine;
9-[2-chloro-4-(trifluoromethyl)phenyl]-2-methoxy-6,7-dimethylimidazo[1,5-a]pyrido[3,2-e]pyrazine;
9-(2-fluoro-5-methylphenyl)-2-methoxy-6,7-dimethylimidazo[1,5-a]pyrido[3,2-e]pyrazine;
9-(2-chloro-5-fluorophenyl)-2-methoxy-6,7-dimethylimidazo[1,5-a]pyrido[3,2-e]pyrazine;
9-[2-chloro-5-(trifluoromethyl)phenyl]-2-methoxy-6,7-dimethylimidazo[1,5-a]pyrido[3,2-e]pyrazine;
9-[2-chloro-5-(trifluoromethoxy)phenyl]-2-methoxy-6,7-dimethylimidazo[1,5-a]pyrido[3,2-e]pyrazine;
4-chloro-3-(2-methoxy-6,7-dimethylimidazo[1,5-a]pyrido[3,2-e]pyrazin-9-yl)benzonitrile;
9-(2-chloro-5-ethoxyphenyl)-2-methoxy-6,7-dimethylimidazo[1,5-a]pyrido[3,2-e]pyrazine;
2-methoxy-6,7-dimethyl-9-pyrimidin-5-ylimidazo[1,5-a]pyrido[3,2-e]pyrazine;
2-methoxy-9-(6-methoxypyridin-3-yl)-6,7-dimethylimidazo[1,5-a]pyrido[3,2-e]pyrazine;
2-methoxy-9-(2-methoxypyridin-3-yl)-6,7-dimethylimidazo[1,5-a]pyrido[3,2-e]pyrazine;
2-methoxy-9-(4-methoxypyridin-3-yl)-6,7-dimethylimidazo[1,5-a]pyrido[3,2-e]pyrazine;
9-(6-fluoro-2-methylpyridin-3-yl)-2-methoxy-6,7-dimethylimidazo[1,5-a]pyrido[3,2-e]pyrazine;
2-methoxy-6,7-dimethyl-9-(4-methylpyridin-3-yl)imidazo[1,5-a]pyrido[3,2-e]pyrazine;
9-(6-fluoro-5-methylpyridin-3-yl)-2-methoxy-6,7-dimethylimidazo[1,5-a]pyrido[3,2-e]pyrazine;
2-methoxy-9-(4-methoxypyridin-3-yl)-6-methyl-7-(trifluoromethyl)imidazo[1,5-a]pyrido[3,2-e]pyrazine;
9-(2,5-dichlorophenyl)-2-methoxy-6-methyl-7-(trifluoromethyl)imidazo[1,5-a]pyrido[3,2-e]pyrazine;
4-fluoro-3-[2-methoxy-6-methyl-7-(trifluoromethyl)imidazo[1,5-a]pyrido[3,2-e]pyrazin-9-yl]benzamide;
2-methoxy-6-methyl-9-(2-methylphenyl)-7-(trifluoromethyl)imidazo[1,5-a]pyrido[3,2-e]pyrazine;
2-methoxy-9-(2-methylphenyl)-7-(trifluoromethyl)imidazo[1,5-a]pyrido[3,2-e]pyrazin-6-amine;
N-[2-methoxy-9-(2-methylphenyl)-7-(trifluoromethyl)imidazo[1,5-a]pyrido[3,2-e]pyrazin-6-yl]methanesulfonamide;
9-(2,5-dichlorophenyl)-2-methoxy-7-methylimidazo[1,5-a]pyrido[3,2-e]pyrazine-6-carbonitrile;
2-methoxy-6,7-dimethyl-9-(3,3,3-trifluoropropyl)imidazo[1,5-a]pyrido[3,2-e]pyrazine;
6-azetidin-1-yl-2-methoxy-7-methyl-9-(3,3,3-trifluoropropyl)imidazo[1,5-a]pyrido[3,2-e]pyrazine;
2-methoxy-7-methyl-9-(3,3,3-trifluoropropyl)imidazo[1,5-a]pyrido[3,2-e]pyrazin-6-amine;
N-[2-methoxy-7-methyl-9-(3,3,3-trifluoropropyl)imidazo[1,5-a]pyrido[3,2-e]pyrazin-6-yl]methanesulfonamide;
4-fluoro-3-[2-methoxy-6-methyl-7-(trifluoromethyl)imidazo[1,5-a]pyrido[3,2-e]pyrazin-9-yl]benzamide;
9-(2,5-dichlorophenyl)-2-methoxy-6,7-dimethylimidazo[1,5-a]pyrido[3,2-e]pyrazine;
9-(3-chlorophenyl)-2-methoxy-6,7-dimethylimidazo[1,5-a]pyrido[3,2-e]pyrazine;
2-(2-methoxy-6,7-dimethylimidazo[1,5-a]pyrido[3,2-e]pyrazin-9-yl)benzamide;
2-methoxy-6,7-dimethyl-9-(2-methylphenyl)imidazo[1,5-a]pyrido[3,2-e]pyrazine;
2-methoxy-6,7-dimethyl-9-[2-(trifluoromethyl)phenyl]imidazo[1,5-a]pyrido[3,2-e]pyrazine;
2-methoxy-9-(2-methoxyphenyl)-6,7-dimethylimidazo[1,5-a]pyrido[3,2-e]pyrazine;
2-methoxy-6,7-dimethyl-9-[2-(trifluoromethoxy)phenyl]imidazo[1,5-a]pyrido[3,2-e]pyrazine;
9-(2-isopropoxyphenyl)-2-methoxy-6,7-dimethylimidazo[1,5-a]pyrido[3,2-e]pyrazine;
2-methoxy-9-(4-methoxyphenyl)-6,7-dimethylimidazo[1,5-a]pyrido[3,2-e]pyrazine;
2-methoxy-6,7-dimethyl-9-(3-thienyl)imidazo[1,5-a]pyrido[3,2-e]pyrazine;
2-methoxy-6,7-dimethyl-9-(3-methyl-2-thienyl)imidazo[1,5-a]pyrido[3,2-e]pyrazine;
9-(3-furyl)-2-methoxy-6,7-dimethylimidazo[1,5-a]pyrido[3,2-e]pyrazine;
2-methoxy-6,7-dimethyl-9-(4-methylphenyl)imidazo[1,5-a]pyrido[3,2-e]pyrazine;
9-(2-furyl)-2-methoxy-6,7-dimethylimidazo[1,5-a]pyrido[3,2-e]pyrazine;
9-(3,5-dimethylisoxazol-4-yl)-2-methoxy-6,7-dimethylimidazo[1,5-a]pyrido[3,2-e]pyrazine;
2-methoxy-9-(3-methoxyphenyl)-6,7-dimethylimidazo[1,5-a]pyrido[3,2-e]pyrazine;
2-methoxy-6,7-dimethyl-9-[3-(trifluoromethoxy)phenyl]imidazo[1,5-a]pyrido[3,2-e]pyrazine;
2-methoxy-6,7-dimethyl-9-[4-(trifluoromethoxy)phenyl]imidazo[1,5-a]pyrido[3,2-e]pyrazine;
2-methoxy-6,7-dimethyl-9-(3-methylphenyl)imidazo[1,5-a]pyrido[3,2-e]pyrazine;
2-methoxy-6,7-dimethyl-9-[3-(trifluoromethyl)phenyl]imidazo[1,5-a]pyrido[3,2-e]pyrazine;
2-methoxy-6,7-dimethyl-9-[4-(trifluoromethyl)phenyl]imidazo[1,5-a]pyrido[3,2-e]pyrazine;
2-methoxy-6,7-dimethyl-9-(2-thienyl)imidazo[1,5-a]pyrido[3,2-e]pyrazine;
2-methoxy-6,7-dimethyl-9-(4-methyl-3-thienyl)imidazo[1,5-a]pyrido[3,2-e]pyrazine;
9-biphenyl-2-yl-2-methoxy-6,7-dimethylimidazo[1,5-a]pyrido[3,2-e]pyrazine;
9-biphenyl-3-yl-2-methoxy-6,7-dimethylimidazo[1,5-a]pyrido[3,2-e]pyrazine;
9-biphenyl-4-yl-2-methoxy-6,7-dimethylimidazo[1,5-a]pyrido[3,2-e]pyrazine;
3-(2-methoxy-6,7-dimethylimidazo[1,5-a]pyrido[3,2-e]pyrazin-9-yl)benzonitrile;
4-(2-methoxy-6,7-dimethylimidazo[1,5-a]pyrido[3,2-e]pyrazin-9-yl)benzonitrile;
2-methoxy-6,7-dimethyl-9-(phenylethynyl)imidazo[1,5-a]pyrido[3,2-e]pyrazine;
9-[(4-fluorophenyl)ethynyl]-2-methoxy-6,7-dimethylimidazo[1,5-a]pyrido[3,2-e]pyrazine;
2-methoxy-9-[(4-methoxyphenyl)ethynyl]-6,7-dimethylimidazo[1,5-a]pyrido[3,2-e]pyrazine;
9-(2-chloro-5-methylphenyl)-2-methoxy-6,7-dimethylimidazo[1,5-a]pyrido[3,2-e]pyrazine;
9-(5-chloro-2-methylphenyl)-2-methoxy-6,7-dimethylimidazo[1,5-a]pyrido[3,2-e]pyrazine;
9-(4-chloro-2-methylphenyl)-2-methoxy-6,7-dimethylimidazo[1,5-a]pyrido[3,2-e]pyrazine;
9-(5-fluoro-2-methylphenyl)-2-methoxy-6,7-dimethylimidazo[1,5-a]pyrido[3,2-e]pyrazine;
9-(4-fluoro-2-methylphenyl)-2-methoxy-6,7-dimethylimidazo[1,5-a]pyrido[3,2-e]pyrazine;
9-(5-fluoro-2-methoxyphenyl)-2-methoxy-6,7-dimethylimidazo[1,5-a]pyrido[3,2-e]pyrazine;
9-(5-chloro-2-methoxyphenyl)-2-methoxy-6,7-dimethylimidazo[1,5-a]pyrido[3,2-e]pyrazine;
4-(2-methoxy-6,7-dimethylimidazo[1,5-a]pyrido[3,2-e]pyrazin-9-yl)benzamide;
9-(4-fluoro-2-methoxyphenyl)-2-methoxy-6,7-dimethylimidazo[1,5-a]pyrido[3,2-e]pyrazine;
9-(3-chloro-2-methylphenyl)-2-methoxy-6,7-dimethylimidazo[1,5-a]pyrido[3,2-e]pyrazine;
9-(3-fluoro-2-methylphenyl)-2-methoxy-6,7-dimethylimidazo[1,5-a]pyrido[3,2-e]pyrazine;
9-(2,3-dichlorophenyl)-2-methoxy-6,7-dimethylimidazo[1,5-a]pyrido[3,2-e]pyrazine;
9-(4-chloro-2-methoxyphenyl)-2-methoxy-6,7-dimethylimidazo[1,5-a]pyrido[3,2-e]pyrazine;
9-[4-chloro-2-(trifluoromethyl)phenyl]-2-methoxy-6,7-dimethylimidazo[1,5-a]pyrido[3,2-e]pyrazine;
9-(5-chloro-2-thienyl)-2-methoxy-6,7-dimethylimidazo[1,5-a]pyrido[3,2-e]pyrazine;
3-(2-methoxy-6,7-dimethylimidazo[1,5-a]pyrido[3,2-e]pyrazin-9-yl)benzamide;
2-methoxy-9-[(3-methoxyphenyl)ethynyl]-6,7-dimethylimidazo[1,5-a]pyrido[3,2-e]pyrazine;
9-(cyclohexylethynyl)-2-methoxy-6,7-dimethylimidazo[1,5-a]pyrido[3,2-e]pyrazine;
9-[(2-chlorophenyl)ethynyl]-2-methoxy-6,7-dimethylimidazo[1,5-a]pyrido[3,2-e]pyrazine;
9-(cyclopropylethynyl)-2-methoxy-6,7-dimethylimidazo[1,5-a]pyrido[3,2-e]pyrazine;
2-methoxy-9-[(2-methoxyphenyl)ethynyl]-6,7-dimethylimidazo[1,5-a]pyrido[3,2-e]pyrazine;
2-methoxy-6,7-dimethyl-9-[(2-methylphenyl)ethynyl]imidazo[1,5-a]pyrido[3,2-e]pyrazine;
3-(2-methoxy-6,7-dimethylimidazo[1,5-a]pyrido[3,2-e]pyrazin-9-yl)-N-methylbenzamide;
N-ethyl-3-(2-methoxy-6,7-dimethylimidazo[1,5-a]pyrido[3,2-e]pyrazin-9-yl)benzamide;
N-methoxy-3-(2-methoxy-6,7-dimethylimidazo[1,5-a]pyrido[3,2-e]pyrazin-9-yl)benzamide;
N-isopropyl-3-(2-methoxy-6,7-dimethylimidazo[1,5-a]pyrido[3,2-e]pyrazin-9-yl)benzamide;
3-(2-methoxy-6,7-dimethylimidazo[1,5-a]pyrido[3,2-e]pyrazin-9-yl)-N,N-dimethylbenzamide;
2-methoxy-6,7-dimethyl-9-[3-(piperidin-1-ylcarbonyl)phenyl]imidazo[1,5-a]pyrido[3,2-e]pyrazine;
4-fluoro-3-(2-methoxy-6,7-dimethylimidazo[1,5-a]pyrido[3,2-e]pyrazin-9-yl)benzamide;
4-fluoro-3-(2-methoxy-6,7-dimethylimidazo[1,5-a]pyrido[3,2-e]pyrazin-9-yl)-N-methylbenzamide;
N-(9-cyclohexyl-2-methoxy-7-methylimidazo[1,5-a]pyrido[3,2-e]pyrazin-6-yl)methanesulfonamide;
2-methoxy-7-methyl-9-propylimidazo[1,5-a]pyrido[3,2-e]pyrazine-6-carboxamide;
9-cyclohexyl-2-methoxy-7-methylimidazo[1,5-a]pyrido[3,2-e]pyrazine-6-carbonitrile;
9-(2-chlorophenyl)-2-methoxy-7-methylimidazo[1,5-a]pyrido[3,2-e]pyrazine-6-carbonitrile;
9-(2,4-dichlorophenyl)-2-methoxy-7-methylimidazo[1,5-a]pyrido[3,2-e]pyrazine-6-carbonitrile;
9-(2,4-dichlorophenyl)-2-methoxy-6,7-dimethylimidazo[1,5-a]pyrido[3,2-e]pyrazine;
9-benzyl-2-methoxy-6,7-dimethylimidazo[1,5-a]pyrido[3,2-e]pyrazine;
4-(2-methoxy-6,7-dimethylimidazo[1,5-a]pyrido[3,2-e]pyrazin-9-yl)-3,5-dimethylisoxazole;
9-(2-fluoro-3-methylphenyl)-2-methoxy-6,7-dimethylimidazo[1,5-a]pyrido[3,2-e]pyrazine;
6-(difluoromethyl)-2-methoxy-7-methyl-9-propylimidazo[1,5-a]pyrido[3,2-e]pyrazine;
9-(benzo[d][1,3]dioxol-5-yl)-2-methoxy-6,7-dimethylimidazo[1,5-a]pyrido[3,2-e]pyrazine;
N-(9-cyclohexyl-2-(cyclopropylmethoxy)-7-methylimidazo[1,5-a]pyrido[3,2-e]pyrazin-6-yl)methanesulfonamide;
9-(2-fluorophenyl)-2-methoxy-6,7-dimethylimidazo[1,5-a]pyrido[3,2-e]pyrazine;
N-(9-(2-fluorophenyl)-2-methoxy-7-methylimidazo[1,5-a]pyrido[3,2-e]pyrazin-6-yl)methanesulfonamide;
N-(9-cyclohexyl-2-(cyclopropylmethoxy)-7-methylimidazo[1,5-a]pyrido[3,2-e]pyrazin-6-yl)-N-(cyclopropylmethyl)methanesulfonamide;
2-(cyclopropylmethoxy)-6,7-dimethyl-9-o-tolylimidazo[1,5-a]pyrido[3,2-e]pyrazine;
9-(2-chlorophenyl)-2-methoxy-6,7-dimethylimidazo[1,5-a]pyrido[3,2-e]pyrazine;
2-methoxy-6,7-dimethyl-9-propylimidazo[1,5-a]pyrido[3,2-e]pyrazine 8-oxide; and
N-(9-cyclohexyl-7-methyl-2-oxo-1,2-dihydroimidazo[1,5-a]pyrido[3,2-e]pyrazin-6-yl)methanesulfonamide;
9-cyclohexyl-2-methoxy-7-methylimidazo[1,5-a]pyrido[3,2-e]pyrazin-6-amine; and
9-(2-chlorophenyl)-2-methoxy-7-methylimidazo[1,5-a]pyrido[3,2-e]pyrazin-6-amine,
or N-oxide thereof, or pharmaceutically acceptable salts thereof.

59. The compound of claim 1 selected from:
3-fluoro-5-(2-methoxy-6,7-dimethylimidazo[1,5-a]pyrido[3,2-e]pyrazin-9-yl)benzamide;
2-fluoro-5-(2-methoxy-6,7-dimethylimidazo[1,5-a]pyrido[3,2-e]pyrazin-9-yl)benzamide;
2-chloro-5-(2-methoxy-6,7-dimethylimidazo[1,5-a]pyrido[3,2-e]pyrazin-9-yl)benzamide;
2-chloro-4-(2-methoxy-6,7-dimethylimidazo[1,5-a]pyrido[3,2-e]pyrazin-9-yl)benzamide;
(2-methoxy-6,7-dimethylimidazo[1,5-a]pyrido[3,2-e]pyrazin-9-yl)acetonitrile;
9-(5-chloro-2-methylphenyl)-2-(cyclopropylmethoxy)-6,7-dimethylimidazo[1,5-a]pyrido[3,2-e]pyrazine;
2-(cyclopropylmethoxy)-9-(4-fluoro-2-methylphenyl)-6,7-dimethylimidazo[1,5-a]pyrido[3,2-e]pyrazine;
2-(cyclopropylmethoxy)-9-(3-fluoro-2-methylphenyl)-6,7-dimethylimidazo[1,5-a]pyrido[3,2-e]pyrazine;
9-[4-chloro-2-(trifluoromethyl)phenyl]-2-(cyclopropylmethoxy)-6,7-dimethylimidazo[1,5-a]pyrido[3,2-e]pyrazine;
9-(2-chloro-4-fluorophenyl)-2-(cyclopropylmethoxy)-6,7-dimethylimidazo[1,5-a]pyrido[3,2-e]pyrazine;
2-(cyclopropylmethoxy)-9-(6-methoxypyridin-3-yl)-6,7-dimethylimidazo[1,5-a]pyrido[3,2-e]pyrazine;
2-(cyclopropylmethoxy)-6,7-dimethyl-9-(4-methylpyridin-3-yl)imidazo[1,5-a]pyrido[3,2-e]pyrazine;
2-(cyclopropylmethoxy)-9-(6-fluoro-2-methylpyridin-3-yl)-6,7-dimethylimidazo[1,5-a]pyrido[3,2-e]pyrazine;
4-[2-(cyclopropylmethoxy)-6,7-dimethylimidazo[1,5-a]pyrido[3,2-e]pyrazin-9-yl]benzamide;
3-[2-(cyclopropylmethoxy)-6,7-dimethylimidazo[1,5-a]pyrido[3,2-e]pyrazin-9-yl]benzamide;
5-(2-(cyclopropylmethoxy)-6,7-dimethylimidazo[1,5-a]pyrido[3,2-e]pyrazin-9-yl)-2-fluorobenzamide;
3-[2-(cyclopropylmethoxy)-6,7-dimethylimidazo[1,5-a]pyrido[3,2-e]pyrazin-9-yl]-5-fluorobenzamide;
3-[2-(cyclopropylmethoxy)-6,7-dimethylimidazo[1,5-a]pyrido[3,2-e]pyrazin-9-yl]-4-methylbenzoic acid;
4-[2-(cyclopropylmethoxy)-6,7-dimethylimidazo[1,5-a]pyrido[3,2-e]pyrazin-9-yl]-3-methylbenzoic acid;
3-[2-(cyclopropylmethoxy)-6,7-dimethylimidazo[1,5-a]pyrido[3,2-e]pyrazin-9-yl]benzenesulfonamide;
3-[2-(cyclopropylmethoxy)-6,7-dimethylimidazo[1,5-a]pyrido[3,2-e]pyrazin-9-yl]-4-methylbenzamide;
4-[2-(cyclopropylmethoxy)-6,7-dimethylimidazo[1,5-a]pyrido[3,2-e]pyrazin-9-yl]-3-methylbenzamide;
3-[2-(cyclopropylmethoxy)-6,7-dimethylimidazo[1,5-a]pyrido[3,2-e]pyrazin-9-yl]-4-fluorobenzamide;
6,7-dimethyl-9-o-tolylimidazo[1,5-a]pyrido[3,2-e]pyrazin-2(1H)-one;
2-(2,2-difluoroethoxy)-6,7-dimethyl-9-(2-methylphenyl)imidazo[1,5-a]pyrido[3,2-e]pyrazine;
2-(2-fluoroethoxy)-6,7-dimethyl-9-(2-methylphenyl)imidazo[1,5-a]pyrido[3,2-e]pyrazine;
6,7-dimethyl-9-(2-methylphenyl)-2-(2,2,2-trifluoroethoxy)imidazo[1,5-e]pyrazine;
6,7-dimethyl-9-(2-methylphenyl)-2-(prop-2-yn-1-yloxy)imidazo[1,5-a]pyrido[3,2-e]pyrazine;
2-[(4-fluorobenzyl)oxy]-6,7-dimethyl-9-(2-methylphenyl)imidazo[1,5-a]pyrido[3,2-e]pyrazine;
6,7-dimethyl-9-(2-methylphenyl)-2-(pyridin-4-ylmethoxy)imidazo[1,5-a]pyrido[3,2-e]pyrazine;
6,7-Dimethyl-9-(4-methylpyridin-3-yl)imidazo[1,5-a]pyrido[3,2-e]pyrazin-2(1H)-one;
2-methoxy-6,7-dimethyl-9-(3-methylpyridin-4-yl)imidazo[1,5-a]pyrido[3,2-e]pyrazine;
2-methoxy-9-(3-methoxypyridin-4-yl)-6,7-dimethylimidazo[1,5-a]pyrido[3,2-e]pyrazine;
2-methoxy-6,7-dimethyl-9-(6-methylpyridin-3-yl)imidazo[1,5-a]pyrido[3,2-e]pyrazine;
2-methoxy-6,7-dimethyl-9-(2-methylpyridin-3-yl)imidazo[1,5-a]pyrido[3,2-e]pyrazine;
9-Bromo-2-methoxy-7-(trifluoromethyl)imidazo[1,5-a]pyrido[3,2-e]pyrazine-6-carbonitrile;
2-methoxy-7-methyl-9-(2-methylphenyl)imidazo[1,5-a]pyrido[3,2-e]pyrazine-6-carbonitrile;
3-(6-cyano-2-methoxy-7-methylimidazo[1,5-a]pyrido[3,2-e]pyrazin-9-yl)-4-fluorobenzamide;
3-(6-cyano-2-methoxy-7-methylimidazo[1,5-a]pyrido[3,2-e]pyrazin-9-yl)benzamide;
5-(6-cyano-2-methoxy-7-methylimidazo[1,5-a]pyrido[3,2-e]pyrazin-9-yl)-2-fluorobenzamide;
2-methoxy-7-methyl-9-(4-methylpyridin-3-yl)imidazo[1,5-a]pyrido[3,2-e]pyrazine-6-carbonitrile;
2-methoxy-7-methyl-9-pyridin-4-ylimidazo[1,5-a]pyrido[3,2-e]pyrazine-6-carbonitrile;
2-methoxy-7-methyl-9-pyridin-3-ylimidazo[1,5-a]pyrido[3,2-e]pyrazine-6-carbonitrile;
9-(6-fluoro-2-methylpyridin-3-yl)-2-methoxy-7-methylimidazo[1,5-a]pyrido[3,2-e]pyrazine-6-carbonitrile;
9-(3,5-dimethyl-1H-pyrazol-4-yl)-2-methoxy-6,7-dimethylimidazo[1,5-a]pyrido[3,2-e]pyrazine;
9-(2-fluoropyridin-4-yl)-2-methoxy-6,7-dimethylimidazo[1,5-a]pyrido[3,2-e]pyrazine;
9-(2-fluoropyridin-3-yl)-2-methoxy-6,7-dimethylimidazo[1,5-a]pyrido[3,2-e]pyrazine;
9-(3-chloropyridin-4-yl)-2-methoxy-6,7-dimethylimidazo[1,5-a]pyrido[3,2-e]pyrazine;
9-(1H-indol-5-yl)-2-methoxy-6,7-dimethylimidazo[1,5-a]pyrido[3,2-e]pyrazine;
5-(2-methoxy-6,7-dimethylimidazo[1,5-a]pyrido[3,2-e]pyrazin-9-yl)-N,N-dimethylpyridin-2-amine;
2-methoxy-6,7-dimethyl-9-(1H-pyrazol-4-yl)imidazo[1,5-a]pyrido[3,2-e]pyrazine;
2-methoxy-6,7-dimethyl-9-(1-methyl-1H-pyrazol-4-yl)imidazo[1,5-a]pyrido[3,2-e]pyrazine;
2-methoxy-6,7-dimethyl-9-(1H-pyrrol-3-yl)imidazo[1,5-a]pyrido[3,2-e]pyrazine;
2-methoxy-6,7-dimethyl-9-[1-(2-methylpropyl)-1H-pyrazol-4-yl]imidazo[1,5-yl}imidazo[1,5-a]pyrido[3,2-e]pyrazine;
9-(2,4-dimethyl-1,3-thiazol-5-yl)-2-methoxy-6,7-dimethylimidazo[1,5-a]pyrido[3,2-e]pyrazine;
2-methoxy-9-(5-methoxypyridin-3-yl)-6,7-dimethylimidazo[1,5-a]pyrido[3,2-e]pyrazine;
2-methoxy-6,7-dimethyl-9-(1-methyl-1H-pyrrol-2-yl)imidazo[1,5-a]pyrido[3,2-e]pyrazine;
9-(4-chloropyridin-3-yl)-2-methoxy-6,7-dimethylimidazo[1,5-a]pyrido[3,2-e]pyrazine;
2-methoxy-6,7-dimethyl-9-(6-morpholin-4-ylpyridin-3-yl)imidazo[1,5-a]pyrido[3,2-e]pyrazine;
2-methoxy-6,7-dimethyl-9-(3-morpholin-4-ylphenyl)imidazo[1,5-a]pyrido[3,2-e]pyrazine;
2-methoxy-6,7-dimethyl-9-(1-propyl-1H-pyrazol-4-yl)imidazo[1,5-a]pyrido[3,2-e]pyrazine;
2-methoxy-6,7-dimethyl-9-[1-(2-morpholin-4-ylethyl)-1H-pyrazol-4-yl]imidazo[1,5-a]pyrido[3,2-e]pyrazine; and
2-methoxy-6,7-dimethyl-9-(1,3,5-trimethyl-1H-pyrazol-4-yl)imidazo[1,5-a]pyrido[3,2-e]pyrazine;
or N-oxide thereof, or pharmaceutically acceptable salt thereof.

60. A pharmaceutical composition comprising a compound of claim 1, or N-oxide thereof, or pharmaceutically acceptable salt thereof, and at least one pharmaceutically acceptable carrier.

61. The composition of claim 60 further comprising at least one pharmaceutically active compound useful in the treatment of central nervous system disorders.

62. The composition of claim 60 wherein said the treatment is not based on PDE10 inhibition.

63. A method of treating or preventing disorders caused by, associated with and/or accompanied by phosphodiesterase 10 hyperactivity and/or disorders in a patient in need thereof, the method comprising administering to said patient a therapeutically effective amount of a compound of claim 1, or N-oxide thereof, or pharmaceutically acceptable salt thereof.

64. A method of treating or preventing central nervous system disorders in a patient in need thereof, the method comprising administering to said patient a therapeutically effective amount of a compound of claim 1, or N-oxide thereof, or pharmaceutically acceptable salt thereof.

65. The method of claim 64 wherein the disorders are selected from neurological disorders and psychiatric disorders; schizophrenia and other psychotic disorders; mood disorders; neurotic, stress-related and somatoform disorders; anxiety disorders; eating disorders; sexual dysfunction; excessive sexual drive; disorders of adult personality and behavior; disorders usually first diagnosed in infancy, childhood or adolescence; mental retardation; disorders of psychological development; disorders comprising the symptom of cognitive deficiency in a mammal, and factitious disorders.

66. The method of claim 64 wherein the disorders are movement disorders with malfunction of basal ganglia selected from focal dystonias; multiple-focal or segmental dystonias; torsion dystonias; hemispheric, generalised and tardive dyskinesias; akathisias; dyskinesias; Huntington's disease; Parkinson's disease; Lewis body disease; restless leg syndrome; and PLMS.

67. The method of claim 64 wherein the disorders are organic disorders selected from symptomatic mental disorders; organic delusional (schizophrenia-like) disorders; presenil or senile psychosis associated with dementia; psychosis in epilepsy and Parkinson's disease and other organic and symptomatic psychosis; delirium; infective psychosis; and personality and behavioural disorders due to brain disease, damage and dysfunction.

68. The method of claim 64 wherein the disorders are selected from mental and behavioural disorders due to psychoactive compounds, more particular to the treatment of psychotic disorders and residual and late-onset psychotic disorders induced by alcohol, opioids, cannabinoids, cocaine, hallucinogens, other stimulants, including caffeine, volatile solvents and other psychoactive compounds.

69. The method of claim 64 further improving learning and memory capacities in a mammal.

70. The method of claim 65 wherein the neurological disorders are selected from neurodegenerative disorders; neurodegeneration associated with cerebral trauma; neurodegeneration associated with stroke; neurodegeneration associated with cerebral infarct; hypoglycemia-induced neurodegeneration; neurodegeneration associated with epileptic seizure; and neurodegeneration associated with neurotoxic poisoning or multi-system atrophy.

71. The method of claim 70 wherein said neurodegenerative disorders are selected from Parkinson's disease, Huntington's disease, and dementia.

72. The method of claim 70 wherein the dementia is selected from Alzheimer's disease, multi-infarct dementia, AIDS-related dementia, and fronto temperal dementia.

73. The method of claim 65 wherein the schizophrenia and other psychotic disorders are selected from continuous or episodic schizophrenia of different types; schizotypal disorders; persistent delusional disorders; acute, transient and persistent psychotic disorders; induced delusional disorders; schizoaffective disorders of different types; puerperal psychosis, and other nonorganic psychosis.

74. The method of claim 65 wherein the mood disorders are selected from manic episodes associated with bipolar disorder and single manic episodes; hypomania; mania with psychotic symptoms; bipolar affective disorders; depressive disorders; single episode or recurrent major depressive disorder; depressive disorder with postpartum onset; depressive disorders with psychotic symptoms; persistent mood disorders; cyclothymia; dysthymia; and premenstrual dysphoric disorder.

75. The method of claim 65 wherein the neurotic, stress-related and somatoform disorders are selected from phobic anxiety disorders; agoraphobia and social phobia related to psychosis; anxiety disorders; panic disorders; general anxiety disorders; obsessive compulsive disorder; reaction to severe stress and adjustment disorders; post traumatic stress disorder; dissociative disorders; neurotic disorders; and depersonalisation-derealisation syndrome.

76. The method of claim 65 wherein the disorders of adult personality and behaviour are selected from specific personality disorders of the paranoid, schizoid, schizotypal, antisocial, borderline, histrionic, narcissistic, avoidant, dissocial, emotionally unstable, anankastic, anxious and dependent type; mixed personality disorders; habit and impulse disorders; and disorders of sexual preference.

77. The method of claim 65 wherein the disorders usually first diagnosed in infancy, childhood or adolescence are selected from hyperkinetic disorders; attentional deficit/hyperactivity disorder (AD/HD); conduct disorders; mixed disorders of conduct and emotional disorders; nonorganic enuresis; nonorganic encopresis; stereotyped movement disorder; and specified behavioural emotional disorders; attention deficit disorder without hyperactivity; excessive masturbation; nail-biting; nose-picking and thumb-sucking; disorders of psychological development; schizoid disorder of childhood; pervasive development disorders; and psychotic episodes associated with Asperger's syndrome.

78. The method of claim 65 wherein the disorders of psychological development are selected from developmental disorders of speech and language; developmental disorders of scholastic skills; specific disorder of arithmetical skills; reading disorders and spelling disorders and other learning disorders, which disorders are predominantly diagnosed in infancy, childhood or adolescence.

79. The method of claim 65 wherein the disorders comprising as a symptom cognitive deficiency are selected from cognitive deficits related to psychosis; age-associated memory impairment; Parkinson's disease; Alzheimer's disease; multi infarct dementia; Lewis body dementia; stroke; frontotemporal dementia; progressive supranuclear palsy Huntington's disease and in HIV disease; cerebral trauma; drug abuse; and mild cognitive disorder.

80. A method of treating or preventing obesity, type 2 diabetes, metabolic syndrome, or glucose intolerance comprising administering to a patient in need a therapeutically effective amount of a compound of claim 1, or N-oxide thereof, or pharmaceutically acceptable salt thereof.

81. The method of claim 80 wherein said patient is overweight or obese.

82. The method of claim 80 wherein the compound is a selective PDE10 inhibitor.

83. The method of claim 80 further comprising administering a further therapeutic agent.

84. The method of claim 83 wherein said further therapeutic agent is an anti-obesity agent.

85. A method of reducing body fat or body weight in a patient comprising administering to said patient in need a therapeutically effective amount of a compound of claim 1, or N-oxide thereof, or pharmaceutically acceptable salt thereof.

86. The method of claim 85 wherein said patient is overweight or obese.

87. The method of claim 85 wherein the compound is a selective PDE10 inhibitor.

88. The method of claim 85 further comprising administering a further therapeutic agent.

89. The method of claim 85 wherein said further therapeutic agent is an anti-obesity agent.

90. A method of treating pain conditions and disorders in a patient comprising administering to said patient in need a therapeutically effective amount of a compound of claim 1, or N-oxide thereof, or pharmaceutically acceptable salt thereof.

91. The method of claim 90 wherein the pain conditions and disorders are selected from inflammatory pain, hyperalgesia, inflammatory hyperalgesia, migraine, cancer pain, osteoarthritis pain, post-surgical pain, non-inflammatory pain, neuropathic pain, peripheral neuropathic pain syndromes, chemotherapy-induced neuropathy, complex regional pain syndrome, HIV sensory neuropathy, neuropathy secondary to tumor infiltration, painful diabetic neuropathy, phantom limb pain, postherpetic neuralgia, postmastectomy pain, trigeminal neuralgia, central neuropathic pain syndromes, central poststroke pain, multiple sclerosis pain, Parkinson disease pain, and spinal cord injury pain.

92. The method of claims 90 wherein the compound or composition, or pharmaceutically acceptable salt thereof, is administered in combination with one or more other agents effective for treating pain.

93. The method of claim 92 wherein the agents are selected from analgesics, non-steroidal anti-inflammatory drugs (NSAIDs), opiods and antidepressants.

94. The method of claim 92 wherein the agents are selected from the group consisting of buprenorphine, naloxone, methadone, levomethadyl acetate, L-alpha acetylmethadol (LAAM), hydroxyzine, diphenoxylate, atropine, chlordiazepoxide, carbamazepine, mianserin, benzodiazepine, phenoziazine, disulfuram, acamprosate, topiramate, ondansetron, sertraline, bupropion, amantadine, amiloride, isradipine, tiagabine, baclofen, propranolol, tricyclic antidepressants, desipramine, carbamazepine, valproate, lamotrigine, doxepin, fluoxetine, imipramine, moclobemide, nortriptyline, paroxetine, sertraline, tryptophan, venlafaxine, trazodone, quetiapine, zolpidem, zopiclone, zaleplon, gabapentin, memantine, pregabalin, cannabinoids, tramadol, duloxetine, milnacipran, naltrexone, paracetamol, metoclopramide, loperamide, clonidine, lofexidine, and diazepam.

95. A process for preparing a compound of claim 1, or N-oxide thereof, or pharmaceutically acceptable salt thereof, the process comprising reacting a compound of Formula (E) 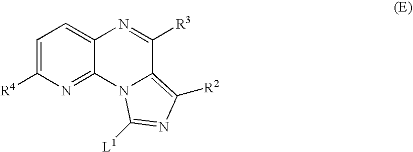
wherein L¹ is halogen;
with R¹—X, wherein X is a leaving group; to prepare said compound of Formula (I).

96. The process of claim 95 wherein said compound of Formula (E) is prepared by the process comprising reacting a compound of Formula (D): 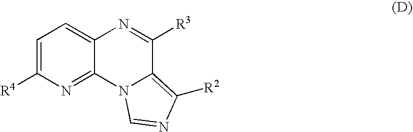
with a halogenating reagent to prepare said compound of Formula (E).

97. The process of claim 96 wherein said compound of Formula (D) is prepared by the process comprising:
a) reacting said compound of Formula (A) 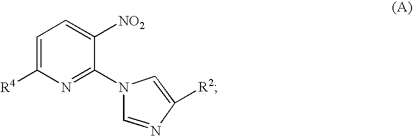
with a reducing agent to prepare a compound of Formula (B) 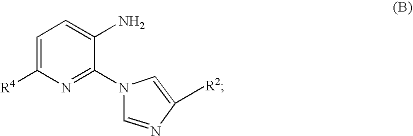
b) reacting a compound of Formula (B) with a compound of Formula: 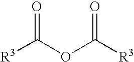
to prepare a compound of Formula (C) 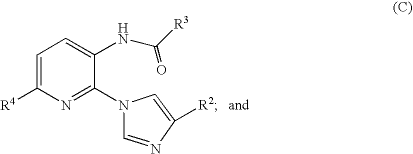
c) reacting said compound of Formula (C) with a cyclizing reagent to prepare said compound of Formula (D).

98. The process of claim 96 wherein said compound of Formula (D) is prepared by the process comprising:
a) reacting a compound of Formula (G) 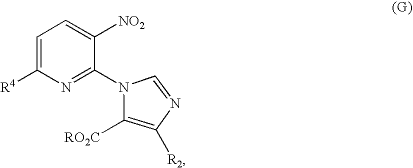
wherein R is C₁₋₄ alkyl; with a reducing agent to prepare a compound of Formula (H) 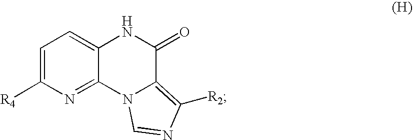
b) reacting a compound of Formula (H) with a halogenating reagent to produce a compound of Formula (J) 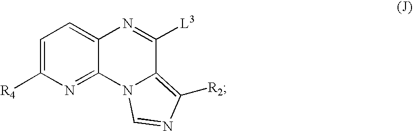
wherein L³ is halogen; and
c) reacting a compound of Formula (J) with an alkylating reagent R³Y, wherein Y is a leaving group; to prepare said compound of Formula (D).

99. A process for preparing a compound of claim 1, or N-oxide thereof, or pharmaceutically acceptable salt thereof, the process comprising:
a) reacting a compound of Formula (D): 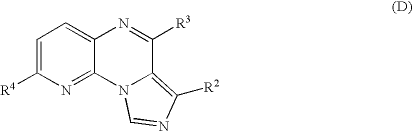
with a halogenating reagent to prepare a compound of Formula (E): 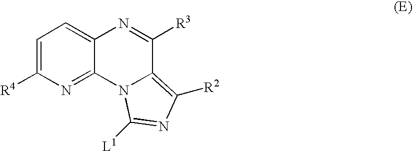
wherein L¹ is a halogen; and
b) reacting a compound of Formula (E) with R¹—X, wherein X is a leaving group; to prepare said compound of formula (I).


SEQUENCE LISTING (14 sequences)

SEQ ID NO: 1 - DNA, 26 bp, Artificial Sequence
  Primer
tgcatctacagggttaccatggagaa

SEQ ID NO: 2 - DNA, 29 bp, Artificial Sequence
  Primer
tatccctgcaggccttcagcagaggctct

SEQ ID NO: 3 - DNA, 28 bp, Artificial Sequence
  Primer
ttcacatggatatgcgacggtaccttct

SEQ ID NO: 4 - DNA, 31 bp, Artificial Sequence
  Primer
ctgtgaagaagaactatcggcgggttcctta

SEQ ID NO: 5 - DNA, 967 bp, Sus scrofa
tgcatctacagggttaccatggagaagctgtcctaccacagcatttgtaccgcggaagagtggcaaggcctcatgcgcttcaaccttcccgtccgtctttgcaaggagattgaattgttccacttcgacattggtccttttgaaaacatgtggcctggaatctttgtctatatggttcatcgcttctgtgggacggcctgctttgagcttgaaaagctgtgtcgttttatcatgtctgtgaagaagaactatcgtcgggttccttaccacaactggaagcacgcggtcacggtggcacactgcatgtacgccatcctccagaacagccacgggctcttcaccgacctcgagcgcaaaggactgctaatcgcgtgtctgtgccacgacctggaccacaggggcttcagcaacagctacctgcagaaattcgaccaccccctggccgctctctactccacgcccaccatggagcagcaccacttctcccagaccgtgtccatcctccagttggaagggcacaacatcttctccaccctgagctccagtgagtacgagcaggtgcttgagatcatccgcaaagccatcattgccacagacctcgctttgtactttggaaacaggaaacagttggaggagatgtaccagaccggatcgctaaaccttaataaccagtcacatagagaccgcgtcattggtttgatgatgactgcctgtgatctctgttccgtgacaaaactgtggccagtaacaaaactgacggcaaatgatatatatgcggaattctgggccgagggcgatgaggtgaagaagctgggaatacagcctattcccatgatggacagagacaagaaggacgaagtcccacaaggccagctcggattctacaacgcggtagctatcccctgctacaccaccctcacccagatcttcccgcccacagagcctcttctgaaggcctgcagggata

SEQ ID NO: 6 - DNA, 732 bp, Guinea pig
ctgtgaagaagaactatcggcgggttccttaccacaactggaagcatgcagtcacggtggcgcactgcatgtacgccatacttcaaaacaacaatggcctcttcacagaccttgagcgcaaaggcctgctaattgcctgtctgtgccatgacctggaccacaggggcttcagtaacagctacctgcagaaattcgaccaccccctggctgcgttgtactccacctccaccatggagcaacaccacttctcccagacggtgttcatcctccagctggaaggacacaacatcttctccaccctgagctccagcgagtacgagcaggtgctggagatcatccgcaaagccatcatcgccactgacctcgcactgtactttgggaacaggaagcagttggaggagatgtaccagacagggtcgctgaacctcaataaccagtcccatcgagaccgcgtcatcggcttgatgatgactgcctgcgatctttgctctgtgacgaaactatggccagttacaaaattgacagcaaatgatatatatgcagagttctgggctgagggggatgagatgaagaagttggggatacagcccatccctatgatggacagagacaagaaggatgaagtccctcaaggacagcttggattctacaatgctgtggccatcccctgctataccaccctgacgcagatcctcccacccacagagcctctgctgaaggcctgcagggata

SEQ ID NO: 7 - DNA, 266 bp, Artificial Sequence
  Synthetically generated oligonucleotide
tagagcctctgctgaaggcctgcagggataacctcaatcagtgggagaaggtaattcgaggggaagagacagcaatgtggatttcaggcccagcaactagcaaaagcacatcagggaagccgaccaggaaggtcgatgactgatcctgaggtgatgtctgcctagcaactgactcaacctgcttctgtgacttcgttctttttatttttatttttttaacggggtgaaaacctctctcagaaggtaccgtcgcatatccatgtgaa

SEQ ID NO: 8 - DNA, 993 bp, Artificial Sequence
  Consensus sequence
acccgagatggcaaggcctcatgccttcaactccgcgttgcggaatgatttccacttgacattggtcctttgaaacatgtggcctggatctttgtctaatgtcatcgttgtgggaccctgttttgacttgaaaatgtgcttttatcatgtctgtgaagaagaactatcggcgggttccttaccacaactggaagcatgcagtcacggtggcgcactgcatgtacgccatacttcaaaacaacaatggcctcttcacagaccttgagcgcaaaggcctgctaattgcctgtctgtgccatgacctggaccacaggggcttcagtaacagctacctgcagaaattcgaccaccccctggctgcgttgtactccacctccaccatggagcaacaccacttctcccagacggtgtccatcctccagctggaaggacacaacatcttctccaccctgagctccagcgagtacgagcaggtgctggagatcatccgcaaagccatcatcgccactgacctcgcactgtactttgggaacaggaagcagttggaggagatgtaccagacagggtcgctgaacctccacaaccagtcccatcgagaccgcgtcatcggcttgatgatgactgcctgcgatctttgctctgtgacgaaactatggccagttacaaaattgacagcaaatgatatatatgcagagttctgggctgagggggatgagatgaagaagttggggatacagcccatccctatgatggacagagacaagcgagatgaagtccctcaaggacagcttggattctacaatgctgtggccatcccctgctataccaccctgacgcagatcctcccacccacagagcctctgctgaaggcctgcagggataacctcaatcagtgggagaaggtaattcgaggggaagagacagcaatgtggatttcaggcccagcaactagcaaaagcacatcggaagccgaccaggaaggtcgatgactga

SEQ ID NO: 9 - DNA, 845 bp, Guinea pig
ctgtgaagaagaactatcgtcgggttccttaccacaactggaagcacgcggtcacggtggcacactgcatgtacgccatcctccagaacagccacgggctcttcaccgacctcgagcgcaaaggactgctaatcgcgtgtctgtgccacgacctggaccacaggggcttcagcaacagctacctgcagaaattcgaccaccccctggccgctctctactccacgcccaccatggagcagcaccacttctcccagaccgtgtccatcctccagttggaagggcacaacatcttctccaccctgagctccagtgagtacgagcaggtgcttgagatcatccgcaaagccatcattgccacagacctcgctttgtactttggaaacaggaaacagttggaggagatgtaccagaccggatcgctaaaccttaataaccagtcacatagagaccgcgtcattggtttgatgatgactgcctgtgatctctgttccgtgacaaaactgtggccagtaacaaaactgacggcaaatgatatatatgcggaattctgggccgagggcgatgaggtgaagaagctgggaatacagcctattcccatgatggacagagacaagaaggacgaagtcccacaaggccagctcggattctacaacgcggtagctatcccctgctacaccaccctcacccagatcttcccgcccacagagcctcttctgaaggcctgcagggataacctcaatcagtgggagaaggtaattcgaggggaagagacagcaatgtggatttcaggcccagcaactagcaaaagcacatcagggaagccgaccaggaaggtcgatgactga

SEQ ID NO: 10 - DNA, 1032 bp, Rattus norvegicus
acctctgaggaatggcaaggcctcatgcacttcaacttgccagcacgcatctgccgggacatcgagctattccactttgacattggtcctttcgagaacatgtggcctgggatctttgtctacatgatccatcggtcttgtgggacatcctgttttgaacttgaaaaattgtgccgttttatcatgtctgtgaagaagaactataggcgggttccttaccacaactggaagcatgcagtcacggtggcgcactgcatgtacgccatacttcaaaacaacaatggcctcttcacagaccttgagcgcaaaggcctgctaattgcctgtctgtgccatgacctggaccacaggggcttcagtaacagctacctgcagaaattcgaccaccccctggctgcgttgtactccacctccaccatggagcaacaccacttctcccagacggtgtccatcctccagctggaaggacacaacatcttctccaccctgagctccagcgagtacgagcaggtgctggagatcatccgcaaagccatcatcgccactgacctcgcactgtactttgggaacaggaagcagttggaggagatgtaccagacagggtcgctgaacctccacaaccagtcccatcgagaccgcgtcatcggcttgatgatgactgcctgcgatctttgctctgtgacgaaactatggccagttacaaaattgacagcaaatgatatatatgcagagttctgggctgagggggatgagatgaagaagttggggatacagcccatccctatgatggacagagacaagcgagatgaagtccctcaaggacagcttggattctacaatgctgtggccatcccctgctataccaccctgacgcagatcctcccacccacagagcctctgctgaaggcctgcagggataacctcaatcagtgggagaaggtaattcgaggggaagagacagcaatgtggatttcaggcccagcaactagcaaaagcacatctgagaagccgaccaggaaggtcgatgactga

SEQ ID NO: 11 - protein, 329 aa, Sus scrofa
IRLCIYRVTMEKLSYHSICTAEEWQGLMRFNLPVRLCKEIELFHFDIGPFENMWPGIFVYMVHRFCGTACFELEKLCRFIMSVKKNYRRVPYHNWKHAVTVAHCMYAILQNSHGLFTDLERKGLLIACLCHDLDHRGFSNSYLQKFDHPLAALYSTPTMEQHHFSQTVSILQLEGHNIFSTLSSSEYEQVLEIIRKAIIATDLALYFGNRKQLEEMYQTGSLNLNNQSHRDRVIGLMMTACDLCSVTKLWPVTKLTANDTYAEPWAEGDEVKKLGIQPIPMMDRDKKDEVPQGQLGFYNAVAIPCYTTLTQIFPPTEPLLKACRDKAEF

SEQ ID NO: 12 - protein, 280 aa, Guinea pig
VKKNYRRVPYHNWKHAVTVAHCMYAILQNSHGLFTDLERKGLLIACLCHDLDHRGFSNSYLQKFDHPLAALYSTSTMEQHHFSQTVFILQLEGHNIFSTLSSSEYEQVLEIIRKAIIATDLALYFGNRKQLEEMYQTGSLNLNNQSHRDRVIGLMMTACDLCSVTKLWPVTKLTANDTYAEPWAEGDEMKKLGIQPIPMMDRDKKDEVPQGQLGFYNAVAIPCYTTLTQILPPTEPLLKACRDNLNQWEKVIRGEETAMWISGPATSKSTSEKPTRKVDD

SEQ ID NO: 13 - protein, 343 aa, Rattus norvegicus
TSEEWQGLMHFNLPARICRDIELFHFDIGPFENMWPGIFVYMIHRSCGTSCFELEKLCRFIMSVKKNYRRVPYHNWKHAVTVAHCMYAILQNNNGLFTDLERKGLLIACLCHDLDHRGFSNSYLQKFDHPLAALYSTSTMEQHHFSQTVSILQLEGHNIFSTLSSSEYEQVLEIIRKAIIATDLALYFGNRKQLEEMYQTGSLNLHNQSHRDRVIGLMMTACDLCSVTKLWPVTKLTANDIYAEFWAEGDEMKKLGIQPIPMMDRDKRDEVPQGQLGFYNAVAIPCYTTLTQILPPTEPLLKACRDNLNQWEKVIRGEETAMWISGPATSKSTSEKPTRKVDD

SEQ ID NO: 14 - protein, 339 aa, Artificial Sequence
  Consensus sequence
TAEEWQGLMFNLPRICKDIELFHFDIGPFENMWPGIFVYMIHRCGTSCFELEKLCRFIMSVKKNYRRVPYHNWKHAVTVAHCMYAILQNNNGLFTDLERKGLLIACLCHDLDHRGFSNSYLQKFDHPLAALYSTSTMEQHHFSQTVSILQLEGHNIFSTLSSSEYEQVLEIIRKAIIATDLALYFGNRKQLEEMYQTGSLNLHNQSHRDRVIGLMMTACDLCSVTKLWPVTKLTANDIYAEFWAEGDEMKKLGIQPIPMMDRDKKDEVPQGQLGFYNAVAIPCYTTLTQILPPTEPLLKACRDNLNQWEKVIRGEETAMWISGPATSKSTSKPTRKVDD